SEQUENCE LISTING (204 sequences)

SEQ ID NO: 1 - protein, 158 aa, human
HHLLASDEEIQDVSGTWYLKAMTVDREFPEMNLESVTPMTLTTLEGGNLEAKVTMLISGRCQEVKAVLEKTDEPGKYTADGGKHVAYIIRSHVKDHYIFYCEGELHGKPVRGVKLVGRDPKNNLEALEDFEKAAGARGLSTESILIPRQSETCSPGSD

SEQ ID NO: 2 - protein, 178 aa, human
QDSTSDLIPAPPLSKVPLQQNFQDNQFQGKWYVVGLAGNAILREDKDPQKMYATIYELKEDKSYNVTSVLFRKKKCDYWIRTFVPGCQPGEFTLGNIKSYPGLTSYLVRVVSTNYNQHAMVFFKKVSQNREYFKITLYGRTKELTSELKENFIRFSKSLGLPENHIVFPVPIDQCIDG

SEQ ID NO: 3 - protein, 535 aa, human
QPPPPPPDATCHQVRSFFQRLQPGLKWVPETPVPGSDLQVCLPKGPTCCSRKMEEKYQLTARLNMEQLLQSASMELKFLIIQNAAVFQEAFEIVVRHAKNYTNAMFKNNYPSLTPQAFEFVGEFFTDVSLYILGSDINVDDMVNELFDSLFPVIYTQLMNPGLPDSALDINECLRGARRDLKVFGNFPKLIMTQVSKSLQVTRIFLQALNLGIEVINTTDHLKFSKDCGRMLTRMWYCSYCQGLMMVKPCGGYCNVVMQGCMAGVVEIDKYWREYILSLEELVNGMYRIYDMENVLLGLFSTIHDSIQYVQKNAGKLTTTIGKLCAHSQQRQYRSAYYPEDLFIDKKVLKVAHVEHEETLSSRRRELIQKLKSFISFYSALPGYICSHSPVAENDTLCWNGQELVERYSQKAARNGMKNQFNLHELKMKGPEPVVSQIIDKLKHINQLLRTMSMPKGRVLDKNLDEEGFESGDCGDDEDECIGGSGDGMIKVKNQLRFLAELAYDLDVDDAPGNSQQATPKDNEISTFHNLGNVH

SEQ ID NO: 4 - protein, 232 aa, human
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 5 - protein, 535 aa, Macaca fascicularis
QPPPPPPDATCHQVRSFFQRLQPGLKWVPETPVPGSDLQVCLPKGPTCCSRKMEEKYQLTARLNMEQLLQSASMELKFLIIQNAAVFQEAFEIVVRHAKNYTNAMFKNNYPSLTPQAFEFVGEFFTDVSLYILGSDINVDDMVNELFDSLFPVIYTQLMNPGLPDSALDINECLRGARRDLKVFGNFPKLIMTQVSKSLQVTRIFLQALNLGIEVINTTDHLKFSKDCGRMLTRMWYCSYCQGLMMVKPCGGYCNVVMQGCMAGVVEIDKYWREYILSLEELVNGMYRIYDMENVLLGLFSTIHDSIQYVQKNAGKLTTTIGKLCAHSQQRQYRSAYYPEDLFIDKKVLKVAHVEHEETLSSRRRELIQKLKSFISFYSALPGYICSHSPVAENDTLCWNGQELVERYSQKAARNGMKNQFNLHELKMKGPEPVVSQIIDKLKHINQLLRTMSVPKGRVLDKNLDEEGFESGDCGDDEDECIGGSGDGMMKVKNQLRFLAELAYDLDVDDVPGNNQQATPKDNEISTFHNLGNVH

SEQ ID NO: 6 - protein, 231 aa, Macaca fascicularis
LQDLCSNCPAGTFCDNNRSQICSPCPPNSFSSAGGQRTCDICRQCKGVFKTRKECSSTSNAECDCISGYHCLGAECSMCEQDCKQGQELTKKGCKDCCFGTFNDQKRGICRPWTNCSLDGKSVLVNGTKERDVVCGPSPADLSPGASSATPPAPAREPGHSPQIIFFLALTSTVVLFLLFFLVLRFSVVKRSRKKLLYIFKQPFMRPVQTTQEEDGCSCRFPEEEEGGCEL

SEQ ID NO: 7 - protein, 152 aa, Artificial Sequence
  lipocalin mutein
ASDEEIQDVSGTWYLKAMTVDRECPEMNLESVTPMTLTTLEGGNLEAKVTMLISGRSQEVKAVLEKTDEPGKYTADGGKHVAYIIRSHVKDHYIFYSEGECHGKPVPGVWLVGRDPKNNLEALEDFEKAAGARGLSTESILIPRQSETSSPG

SEQ ID NO: 8 - protein, 178 aa, Artificial Sequence
  lipocalin mutein
QDSTSDLIPAPPLSKVPLQQNFQDNQFHGKWYVVGLAGNAILREDKDPQKMYATIYELKEDKSYNVTSVLFRKKKCDYWIRTFVPGSQPGEFTLGNIKSYPGLTSYLVRVVSTNYNQHAMVFFKKVSQNREYFKITLYGRTKELTSELKENFIRFSKSLGLPENHIVFPVPIDQCIDG

SEQ ID NO: 9 - protein, 8 aa, Artificial Sequence
  affinity tag
WSHPQFEK

SEQ ID NO: 10 - protein, 10 aa, Artificial Sequence
  affinity tag
HHHHHHHHHH

SEQ ID NO: 11 - protein, 10 aa, Artificial Sequence
  affinity tag
EQKLISEEDL

SEQ ID NO: 12 - protein, 15 aa, Artificial Sequence
  linker
GGGGSGGGGSGGGGS

SEQ ID NO: 13 - protein, 18 aa, Artificial Sequence
  linker
PSTPPTNSSSTPPTPSPS

SEQ ID NO: 14 - protein, 14 aa, Artificial Sequence
  linker
GGSGNSSGSGGSPV

SEQ ID NO: 15 - protein, 20 aa, Artificial Sequence
  linker
ASPAAPAPASPAAPAPSAPA

SEQ ID NO: 16 - protein, 66 aa, Artificial Sequence
  linker
AGSGGSGGSGGSPVPSTPPTPSPSTPPTPSPSGGSGNSSGSGGSPVPSTPPTPSPSTPPTPSPSAS

SEQ ID NO: 17 - protein, 32 aa, Artificial Sequence
  linker
PSTPPTPSPSTPPTPSPSGGSGNSSGSGGSPV

SEQ ID NO: 18 - protein, 74 aa, Artificial Sequence
  linker
AGSGGSGGSGGSPVPSTPPTNSSSTPPTPSPSPVPSTPPTNSSSTPPTPSPSPVPSTPPTNSSSTPPTPSPSAS

SEQ ID NO: 19 - protein, 40 aa, Artificial Sequence
  linker
ASPAAPAPASPAAPAPSAPAASPAAPAPASPAAPAPSAPA

SEQ ID NO: 20 - protein, 10 aa, Artificial Sequence
  linker
VDDIEGRMDE

SEQ ID NO: 21 - protein, 11 aa, Artificial Sequence
  linker
ENLYFQGRMDE

SEQ ID NO: 22 - protein, 10 aa, Artificial Sequence
  linker
GGGGSGGGGS

SEQ ID NO: 23 - protein, 5 aa, Artificial Sequence
  linker
GGGGS

SEQ ID NO: 24 - protein, 20 aa, Artificial Sequence
  linker
GGGGSGGGGSGGGGSGGGGS

SEQ ID NO: 25 - protein, 25 aa, Artificial Sequence
  linker
GGGGSGGGGSGGGGSGGGGSGGGGS

SEQ ID NO: 26 - protein, 7 aa, Artificial Sequence
  linker
GGSGGAP

SEQ ID NO: 27 - protein, 15 aa, Artificial Sequence
  linker
GSPTPTPTTPTPTPT

SEQ ID NO: 28 - protein, 17 aa, Artificial Sequence
  linker
AEAAAKEAAAKEAAAKA

SEQ ID NO: 29 - protein, 327 aa, human
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 214 aa, human
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 228 aa, Artificial Sequence
  antibody Fc region
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 32 - protein, 228 aa, Artificial Sequence
  antibody Fc region
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 33 - protein, 228 aa, Artificial Sequence
  antibody Fc region
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 34 - protein, 228 aa, Artificial Sequence
  antibody Fc region
ESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 35 - protein, 51 aa, Artificial Sequence
  multimerization domain
GVRLWATRQAMLGQVHEVPEGWLIFVAEQEELYVRVQNGFRKVQLEARTPL

SEQ ID NO: 36 - protein, 39 aa, Artificial Sequence
  multimerization domain
KPLDGEYFTLQIRGRERFEMFRELNEALELKDAQAGKEP

SEQ ID NO: 37 - protein, 33 aa, Artificial Sequence
  multimerization domain
RLKQIEDKLEEILSKLYHIENELARIKKLLGER

SEQ ID NO: 38 - protein, 244 aa, Artificial Sequence
  fusion protein
QDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDGGGGGSGGGGSGGGGSGVRLWATRQAMLGQVHEVPEGWLIFVAEQEELYVRVQNGFRKVQLEARTPL

SEQ ID NO: 39 - protein, 234 aa, Artificial Sequence
  fusion protein
QDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDGGGGGSGVRLWATRQAMLGQVHEVPEGWLIFVAEQEELYVRVQNGFRKVQLEARTPL

SEQ ID NO: 40 - protein, 254 aa, Artificial Sequence
  fusion protein
QDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDGGGGGSGGGGSGGGGSGGGGSGGGGSGVRLWATRQAMLGQVHEVPEGWLIFVAEQEELYVRVQNGFRKVQLEARTPL

SEQ ID NO: 41 - protein, 244 aa, Artificial Sequence
  fusion protein
QDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDGGSPTPTPTTPTPTPTGVRLWATRQAMLGQVHEVPEGWLIFVAEQEELYVRVQNGFRKVQLEARTPL

SEQ ID NO: 42 - protein, 246 aa, Artificial Sequence
  fusion protein
QDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDGAEAAAKEAAAKEAAAKAGVRLWATRQAMLGQVHEVPEGWLIFVAEQEELYVRVQNGFRKVQLEARTPL

SEQ ID NO: 43 - protein, 244 aa, Artificial Sequence
  fusion protein
GVRLWATRQAMLGQVHEVPEGWLIFVAEQEELYVRVQNGFRKVQLEARTPLGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 44 - protein, 234 aa, Artificial Sequence
  fusion protein
GVRLWATRQAMLGQVHEVPEGWLIFVAEQEELYVRVQNGFRKVQLEARTPLGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 45 - protein, 254 aa, Artificial Sequence
  fusion protein
GVRLWATRQAMLGQVHEVPEGWLIFVAEQEELYVRVQNGFRKVQLEARTPLGGGGSGGGGSGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 46 - protein, 226 aa, Artificial Sequence
  fusion protein
QDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDGGGGGSGGGGSGGGGSRLKQIEDKLEEILSKLYHIENELARIKKLLGER

SEQ ID NO: 47 - protein, 232 aa, Artificial Sequence
  fusion protein
QDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDGGGGGSGGGGSGGGGSKPLDGEYFTLQIRGRERFEMFRELNEALELKDAQAGKEP

SEQ ID NO: 48 - protein, 437 aa, Artificial Sequence
  fusion protein
QDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDGGGGGSGGGGSGGGGSGVRLWATRQAMLGQVHEVPEGWLIFVAEQEELYVRVQNGFRKVQLEARTPLGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 49 - protein, 427 aa, Artificial Sequence
  fusion protein
QDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDGGGGGSGVRLWATRQAMLGQVHEVPEGWLIFVAEQEELYVRVQNGFRKVQLEARTPLGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 50 - protein, 447 aa, Artificial Sequence
  fusion protein
QDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDGGGGGSGGGGSGGGGSGGGGSGGGGSGVRLWATRQAMLGQVHEVPEGWLIFVAEQEELYVRVQNGFRKVQLEARTPLGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 51 - protein, 439 aa, Artificial Sequence
  fusion protein
QDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDGAEAAAKEAAAKEAAAKAGVRLWATRQAMLGQVHEVPEGWLIFVAEQEELYVRVQNGFRKVQLEARTPLGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 52 - protein, 437 aa, Artificial Sequence
  fusion protein
QDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDGGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDGGGGGSGGGGSGGGGSGVRLWATRQAMLGQVHEVPEGWLIFVAEQEELYVRVQNGFRKVQLEARTPL

SEQ ID NO: 53 - protein, 437 aa, Artificial Sequence
  fusion protein
GVRLWATRQAMLGQVHEVPEGWLIFVAEQEELYVRVQNGFRKVQLEARTPLGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDGGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 54 - protein, 507 aa, Artificial Sequence
  fusion protein
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTLVSSGGGGSGGGGSGGGGSGGGGSDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKGGGGSGGGGSGGGGSGVRLWATRQAMLGQVHEVPEGWLIFVAEQEELYVRVQNGFRKVQLEARTPLGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 55 - protein, 437 aa, Artificial Sequence
  fusion protein
QDSTSDLIPAPPLSKVPLQQNFQDNQFQGKWYVVGRAGNVGLREDKDPPKMWATIYELKEDKSYDVTNVRFARKKCTYSIGTFVPGSQPGEFTLGQIKSEPGGTANLVRVVSTNYNQHAMVFFKEVYQNREIFFIILYGRTKELTSELKENFIRFSKSLGLPENHIVFPVPIDQCIDGGGGGSGGGGSGGGGSGVRLWATRQAMLGQVHEVPEGWLIFVAEQEELYVRVQNGFRKVQLEARTPLGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 56 - protein, 152 aa, Artificial Sequence
  lipocalin mutein
ASDEEIQDVSGTWYLKAMTVDEGCRPWNIFSVTPMTLTTLEGGNLEAKVTMAIDGPAQEVKAVLEKTDEPGKYTADGGKHVAYIIRSHVKDHYIFYSEGVCDGSPVPGVWLVGRDPKNNLEALEDFEKAAGARGLSTESILIPRQSETSSPG

SEQ ID NO: 57 - protein, 152 aa, Artificial Sequence
  lipocalin mutein
TSDEEIQDVSGTWYLKAMTVDEGCRPWNIFSVTPMTLTTLEGGNLEAKVTMAIDGPAQEVRAVLEKTDEPGKYTADGGKHDAYIIRSHVKDHYIFYSEGVCDGSPVPGVWLVGRDPENNLEALEDFEKTAGARGLSTESILIPRQSETSSPG

SEQ ID NO: 58 - protein, 152 aa, Artificial Sequence
  lipocalin mutein
ASDEEIQDVSGTWYLKAMTVDEGCRPWNIFSVTPMTLTTLEGGNLEAKVTMAIDGPAQEVNAVLEKTDEPGKYTADGGKHVAYIIRSHVRDHYIFYSEGVCDGSPVPGVWLVGRDPENNLEALEDFEKTAGARGLSTESILIPRQSETSSPG

SEQ ID NO: 59 - protein, 152 aa, Artificial Sequence
  lipocalin mutein
VSDEEIQDVSGTWYLKAMTVDEGCRPWNIFSVTPMTLTTLEGGNLEAKVTMAIDGPAQEVRAVLEKTDEPGKYTADGGKHVAYIIRSHVEDHYIFYSEGVCDGSPVPGVWLVGRDPENNLEALEDFEKTAGARGLSTESILIPRQSETSSPG

SEQ ID NO: 60 - protein, 152 aa, Artificial Sequence
  lipocalin mutein
ASDEEIQDVSGTWYLKAMTVDEGCRPWNIFSVTPMTLSTLEGGNLEAKVTMAIDGPAQEVKAVLEKTDEPGKYTADGGKHVAYIIRSHVKDHYIFYSEGVCDGSPVPGVWLVGRDPKNNLEALEDFEKAAGARGLSTESILIPRQIETSSPG

SEQ ID NO: 61 - protein, 152 aa, Artificial Sequence
  lipocalin mutein
ASDEEIQDVSGTWYLKAMTVDEGCRPWNIFSVTPMTLTTLEGGNLEAEVTMAIDGPAQEVKAVLEKADEPGKYTADGGKHVAYIIRSHVKDHYIFYSEGVCDGSPVPGVWLVGRDPKNNLEALEDFEKTAGARGLSTESILIPSQIETSSPG

SEQ ID NO: 62 - protein, 152 aa, Artificial Sequence
  lipocalin mutein
TSDEEIQDVSGTWYLKAMTVDEGCRPWNIFSVTPMTLTTLEDGNLEAKVTMAIDGPAQEVKAVLEKADEPGKYTADGGKHVAYIIRSHVKDHYIFYSEGVCDGSPVPGVWLVGRDPKNNLEALEDFEKAAGARGLSTESILIPRQIETSSPG

SEQ ID NO: 63 - protein, 178 aa, Artificial Sequence
  lipocalin mutein
QDSTSDLIPAPPLSKVPLQQNFQDNQFHGKWYVVGQAGNIKLREDKDPNKMMATIYELKEDKSYNVTGVTFDDKKCTYAISTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 64 - protein, 178 aa, Artificial Sequence
  lipocalin mutein
QDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 65 - protein, 178 aa, Artificial Sequence
  lipocalin mutein
QDSTSDLIPAPPLSKVPLQQNFQDNQFHGKWYVVGQAGNIRLREDKDPNKMMATIYELKEDKSYDVTAVAFDDKKCTYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 66 - protein, 178 aa, Artificial Sequence
  lipocalin mutein
QDSTSDLIPAPPLSKVPLQQNFQDNQFHGKWYVVGQAGNIKLREDKDPNKMMATIYELKEDKSYDVTAVAFDDKKCTYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 67 - protein, 175 aa, Artificial Sequence
  lipocalin mutein
QDSTSDLIPAPPLSKVPLQQNFQDNQFHGKWYVVGQAGNIKLREDSKMMATIYELKEDKSYDVTGVSFDDKKCTYAIMTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 68 - protein, 178 aa, Artificial Sequence
  lipocalin mutein
QDSTSDLIPAPPLSKVPLQQNFQDNQFHGKWYVVGQAGNIKLREDKDPVKMMATIYELKEDKSYDVTGVTFDDKKCRYDISTFVPGSQPGEFTFGKIKSFPGHTSSLVRVVSTNYNQHAMVFFKFVFQNREEFYITLYGRTKELTSELKENFIRFSKSLGLPENHIVFPVPIDQCIDG

SEQ ID NO: 69 - protein, 178 aa, Artificial Sequence
  lipocalin mutein
QDSTSDLIPAPPLSKVPLQQNFQDNQFHGKWYVVGQAGNIRLREDKDPHKMMATIYELKEDKSYDVTGVTFDDKKCTYAISTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 70 - protein, 178 aa, Artificial Sequence
  lipocalin mutein
QDSTSDLIPAPPLSKVPLQQNFQDNQFHGKWYVVGQAGNIKLREDKDPNKMMATIYELKEDKSYDVTGVTFDDKKCTYAISTLVPGSQPGEFTFGKIKSFPGHTSSLVRVVSTNYNQHAMVFFKFVFQNREEFYITLYGRTKELTSELKENFIRFSKSLGLPENHIVFPVPIDQCIDG

SEQ ID NO: 71 - protein, 178 aa, Artificial Sequence
  lipocalin mutein
QDSTSDLIPAPPLSKVPLQQNFQDNQFHGKWYVVGQAGNIRLREDKDPSKMMATIYELKEDKSYDVTAVTFDDKKCNYAISTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 72 - protein, 448 aa, Artificial Sequence
  antibody heavy chain
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 73 - protein, 216 aa, Artificial Sequence
  antibody light chain
EIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 178 aa, Artificial Sequence
  lipocalin mutein
QDSTSDLIPAPPLSKVPLQQNFQDNQFHGKWYVVGMAGNNLLREDKDPHKMSATIYELKEDKSYNVTDVMFLDKKCQYIIWTFVPGSQPGEFTLGFIKSDPGHTSYLVRVVSTNYNQHAMVFFKSVIQNREWFGITLYGRTKELTSELKENFIRFSKSLGLPENHIVFPVPIDQCIDG

SEQ ID NO: 75 - protein, 178 aa, Artificial Sequence
  lipocalin mutein
QDSTSDLIPAPPLSKVPLQQNFQDNQFQGKWYVVGMAGNNLLREDKDPHKMSATIYELKEDKSYNVTDVMFLDKKCQYIIWTFVPGSQPGELTLGFIRSDLGHTSYLVRVVSTNYNQHAMVFFKSVIQNREWFGITLYGRTKELTSELKENFIRFSKSLGLPENHIVFPVPIDQCIDG

SEQ ID NO: 76 - protein, 178 aa, Artificial Sequence
  lipocalin mutein
QDSTSDLIPAPPLSKVPLQQNFQDYQFQGKWYVVGMAGNNLLREDKDPHKMGATIYELKEDKSYNVTDVMLLDKKCQYIIQTFVPGSQPGESTLGFIKSDPGHTSYLVRVVSTNYNQHAMVFFKSVIQNREWFGITLYGRTKELTSELKENFIRFSKSLGLPENHIVFPVPIDQCIDG

SEQ ID NO: 77 - protein, 178 aa, Artificial Sequence
  lipocalin mutein
QDSTSDLIPAPPLSKVPLQQNFQDNQFQGKWYVVGMAGNNLLREDKDPHKMGATIYELKEDKSYNVTDVMFLDKKCQHIIWTFVPGSQPGELTLGFIKSDPGHTSYLVRVVSTNYNQHAMVFFKSVIQNREWFGITLYGRTKELTSELKENFIRFSKSLGLPENHIVFPVPIDQCIDG

SEQ ID NO: 78 - protein, 178 aa, Artificial Sequence
  lipocalin mutein
QDSTSDLIPAPPLSKVPLQQNFQDDQFQGKWYVVGMAGNNLLREDKDPHKMGATIYELKEDKSYNVTDVMFLDKKCQYIIWTFVPGSQPGELTLGFIKSDPGHTSYLVRVVSTNYNQHAMVFFKSVIQNREWFGITLYGRTKELTSELKENFIRFSKSLGLPENHIVFPVPIDQCIDG

SEQ ID NO: 79 - protein, 178 aa, Artificial Sequence
  lipocalin mutein
QDSTSDLIPAPPLSKVPLQQNFQDNQFQGKWYIVGMAGNNLLREDKDPHKMGATIYELKEDKSYNVTDVMFLDKKCQYIIWTFVPGSQPGELTLGFIKSDPGHTSYLVRVVSTNYNQHAMVFFKSVIQNREWFGITLYGRTKELTSELKENFIRFSKSLGLPENHIVFPVPIDQCIDG

SEQ ID NO: 80 - protein, 178 aa, Artificial Sequence
  lipocalin mutein
QDSTSDLIPAPPLSKVPLQRNFQDNQFQGKWYVVGMAGNNLLRVDKDPHKMGATIYELKEDKSYNVTDVMFLDKKCQYIIWTFVPGSQPGELTLGFIKSDPGHTSYLVRVVSTNYNQHAMVYFKSVIQNREWFGITLYGRTKELTSELKENFIRFSKSLGLPENHIVFPVPIDQCIDG

SEQ ID NO: 81 - protein, 178 aa, Artificial Sequence
  lipocalin mutein
QDSTSDLIPAPPLSKVPLQQNFQDNQFQGKWYVVGMAGNNLLREDKDPHKMSATIYELKEDKSYNVTDVMFLDKKCQYINWPFVPGSQPGEFTLGFIKSDLGPTSYLVRVVSTNYNQHAMVFFKSVIQNREWFGITLYGRTKELTSELKENFIRFSKSLGLPENHIVFPVPIDQCIDG

SEQ ID NO: 82 - protein, 178 aa, Artificial Sequence
  lipocalin mutein
QDSTSDLIPAPPLSKVPLQQNFQDNQFQGKWYVVGMAGNNLLREDKDPHKMGATIYELNEDKSYNVTDVMFLDKKCQYIIWTFVPGSQPGELTLGFIKSDPGHTSYLVRVVSTNYNQHAMVFFKSVIQNREWFGITLYGRTKELTSELKENFIRFSKSLGLPENHIVFPVPIDQCIDG

SEQ ID NO: 83 - protein, 178 aa, Artificial Sequence
  lipocalin mutein
QDSTSDLIPAPPLSKVPLQQNFQDNQFQGKWYVVGMAGNNLLRDDKDPHKMSATIYELKEDKSYNVTDVMLLDKKCHYIIWTFVPGSQPGELTLGFIKSDPGHTSYLVRVVSTNYNQHAMVFFKSVIQNREWFGITLYGRTKELTSELKENFIRFSKSLGLPENHIVFPVPIDQCIDG

SEQ ID NO: 84 - protein, 178 aa, Artificial Sequence
  lipocalin mutein
QDSTSDLIPAPPLSKVPLQQNFQDNQFHGKWYVVGVAGNAMLREDKDPLKMRATIYELKEDKSYNVTGVSFWRKKCHYKIGTFVPGSQPGEFTLGDIKSGPGQTSNLVRVVSTNYNQHAMVFFKEVRQNREWFAITLYGRTKELTSELKENFIRFSKSLGLPENHIVFPVPIDQCIDG

SEQ ID NO: 85 - protein, 178 aa, Artificial Sequence
  lipocalin mutein
QDSTSDLIPAPPLSKVPLQQNFQDNQFHGKWYVVGRAGNVALREDKDPPKMRATIYELKEDKSYNVTNVRFAMKKCMYSIGTFVPGSQPGEFTLGQIKSEPGNTSNLVRVVSTNYNQHAMVFFKEVYQNREIFFITLYGRTKELTSELKENFIRFSKSLGLPENHIVFPVPIDQCIDG

SEQ ID NO: 86 - protein, 178 aa, Artificial Sequence
  lipocalin mutein
QDSTSDLIPAPPLSKVPLQQNFQDNQFQGKWYVVGVAGNALLREDKDPLKMRATIYELKEDKSYDVTVVSFWRKKCHYKIGTFVPGSQPGEFTLGDIKSGPGQTSNLVRVVSTNYNQHAMVFFKEVRQNREWFAITLYGRTKELTSELKENFIRFSKSLGLPENHIVFPVPIDQCIDG

SEQ ID NO: 87 - protein, 178 aa, Artificial Sequence
  lipocalin mutein
QDSTSDLIPAPPLSKVPLQQNFQDNQFQGKWYVVGVAGNVRLREDKDPPKMRATIYELKEDKSYDVTGVSFRGKKCHYKIGTFVPGSQPGEFTLGDIKSGPGETSNLVRVVSTNYNQHAMVFFKEVRQNREWFFITLYGRTKELTSELKENFIRFSKSLGLPENHIVFPVPIDQCIDG

SEQ ID NO: 88 - protein, 178 aa, Artificial Sequence
  lipocalin mutein
QDSTSDLIPAPPLSKVPLQQNFQDNQFQGKWYVVGVAGNGMLREDKDPLKMRATIYELKEDKSYDVTSVAFRNKKCHYKIGTFVPGSQPGEFTLGQIKSGPGETSNLVRVVSTNYNQHAMVFFKEVRQNREWFFITLYGRTKELTSELKENFIRFSKSLGLPENHIVFPVPIDQCIDG

SEQ ID NO: 89 - protein, 178 aa, Artificial Sequence
  lipocalin mutein
QDSTSDLIPAPPLSKVPLQQNFQDNQFQGKWYVVGRAGNVGLREDKDPPKMWATIYELKEDKSYDVTNVRFARKKCLYSIGTFVPGSQPGEFTLGQIKSEPGNTANLVRVVSTNYNQHAMVFFKEVYQNREIFFIILYGRTKELTSELKENFIRFSKSLGLPENHIVFPVPIDQCIDG

SEQ ID NO: 90 - protein, 178 aa, Artificial Sequence
  lipocalin mutein
QDSTSDLIPAPPLSKVPLQQNFQDNQFQGKWYVVGRAGNVGLREDKDPPKMWATIYELKEDKSYDVTNVRFARKKCTYSIGTFVPGSQPGEFTLGQIKSEPGGTANLVRVVSTNYNQHAMVFFKEVYQNREIFFIILYGRTKELTSELKENFIRFSKSLGLPENHIVFPVPIDQCIDG

SEQ ID NO: 91 - protein, 178 aa, Artificial Sequence
  lipocalin mutein
QDSTSDLIPAPPLSKVPLQQNFQDNQFQGKWYVVGRAGNGALREDKDPPKMWATIYELKEDKSYDVTNVRFARKKCVYSIGTFVPGSQPGEFTLGPIKSEPGNTASLVRVVSTNYNQHAMVFFKEVYQNREIFFIILYGRTKELTSELKENFIRFSKSLGLPENHIVFPVPIDQCIDG

SEQ ID NO: 92 - protein, 178 aa, Artificial Sequence
  lipocalin mutein
QDSTSDLIPAPPLSKVPLQQNFQDNQFQGKWYVVGRAGNVALREDKDPPKMRATIYELKEDKSYDVTAVRFARKKCLYSIGTFVPGSQPGEFTLGRIKSEPGYTANLVRVVSTNYNQHAMVFFKEVYQNREIFFIILYGRTKELTSELKENFIRFSKSLGLPENHIVFPVPIDQCIDG

SEQ ID NO: 93 - protein, 178 aa, Artificial Sequence
  lipocalin mutein
QDSTSDLIPAPPLSKVPLQQNFQDNQFQGKWYVVGRAGNVALREDKDPPKMRATIYELKEDKSYDVTNVVFAGKKCKYSIGTFVPGSQPGEFTLGRIKSPPGNTANLVRVVSTNYNQHAMVFFKEVYQNREIFFIILYGRTKELTSELKENFIRFSKSLGLPENHIVFPVPIDQCIDG

SEQ ID NO: 94 - protein, 178 aa, Artificial Sequence
  lipocalin mutein
QDSTSDLIPAPPLSKVPLQQNFQDNQFQGKWYVVGRAGNLGLREDKDPPKMWATIYELKEDKSYDVTNVRFARKKCMYSIGTFVPGSQPGEFTLGQIKSEPGSTANLVRVVSTNYNQHAMVFFKEVYQNREIFFITLYGRTKELTSELKENFIRFSKSLGLPENHIVFPVPIDQCIDG

SEQ ID NO: 95 - protein, 178 aa, Artificial Sequence
  lipocalin mutein
QDSTSDLIPAPPLSKVPLQQNFQDNQFQGKWYVVGRAGNVGLREDKDPPKMWATIYELKEDKSYDVTNVRFAGKKVKYTIGTFVPGSQPGEFTLGQIKSEPGNTATLVRVVSTNYNQHAMVFFKEVYQNREIFFITLYGRTKELTSELKENFIRFSKSLGLPENHIVFPVPIDQAIDG

SEQ ID NO: 96 - protein, 178 aa, Artificial Sequence
  lipocalin mutein
QDSTSDLIPAPPLSKVPLQQNFQDNQFQGKWYVVGRAGNVGLREDKDPPKMRATIYELKEDKSYDVTGVRFGEKKIKYSIGTFVPGSQPGEFTLGQIKSQPGDTANLVRVVSTNYNQHAMVFFKEVYQNREIFFIILYGRTKELTSELKENFIRFSKSLGLPENHIVFPVPIDQAIDG

SEQ ID NO: 97 - protein, 178 aa, Artificial Sequence
  lipocalin mutein
QDSTSDLIPAPPLSKVPLQQNFQDNQFQGKWYVVGRAGNVGLREDKDPPKMRATIYELKEDKSYDVTGVRFDSKKVTYSIGTFVPGSQPGEFTLGQIKSEPGNTANLVRVVSTNYNQHAMVFFKEVYQNREIFFIILYGRTKELTSELKENFIRFSKSLGLPENHIVFPVPIDQAIDG

SEQ ID NO: 98 - protein, 248 aa, Artificial Sequence
  antibody single chain Fv fragment
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTLVSSGGGGSGGGGSGGGGSGGGGSDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIK

SEQ ID NO: 99 - protein, 8 aa, Artificial Sequence
  antibody heavy chain CDR1
GYTFTDYE

SEQ ID NO: 100 - protein, 8 aa, Artificial Sequence
  antibody heavy chain CDR2
LDPKTGDT

SEQ ID NO: 101 - protein, 8 aa, Artificial Sequence
  antibody heavy chain CDR3
TRFYSYTY

SEQ ID NO: 102 - protein, 11 aa, Artificial Sequence
  antibody light chain CDR1
QSLVHSNRNTY

SEQ ID NO: 103 - protein, 9 aa, Artificial Sequence
  antibody light chain CDR3
SQNTHVPPT

SEQ ID NO: 104 - protein, 115 aa, Artificial Sequence
  antibody heavy chain variable region
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSS

SEQ ID NO: 105 - protein, 116 aa, Artificial Sequence
  antibody heavy chain variable region
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTLVSS

SEQ ID NO: 106 - protein, 112 aa, Artificial Sequence
  antibody light chain variable region
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIK

SEQ ID NO: 107 - protein, 445 aa, Artificial Sequence
  antibody heavy chain
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 442 aa, Artificial Sequence
  antibody heavy chain
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTLVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 109 - protein, 219 aa, Artificial Sequence
  antibody light chain
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 8 aa, Artificial Sequence
  antibody heavy chain CDR1
GFTFNKNA

SEQ ID NO: 111 - protein, 10 aa, Artificial Sequence
  antibody heavy chain CDR2
IRNKTNNYAT

SEQ ID NO: 112 - protein, 8 aa, Artificial Sequence
  antibody heavy chain CDR3
VAGNSFAY

SEQ ID NO: 113 - protein, 12 aa, Artificial Sequence
  antibody light chain CDR1
QSLLYSSNQKNY

SEQ ID NO: 114 - protein, 9 aa, Artificial Sequence
  antibody light chain CDR3
QQYYNYPLT

SEQ ID NO: 115 - protein, 116 aa, Artificial Sequence
  antibody heavy chain variable region
EVQLVESGGGLVQPGGSLRLSCAASGFTFNKNAMNWVRQAPGKGLEWVGRIRNKTNNYATYYADSVKARFTISRDDSKNSLYLQMNSLKTEDTAVYYCVAGNSFAYWGQGTLVTVS

SEQ ID NO: 116 - protein, 113 aa, Artificial Sequence
  antibody light chain variable region
DIVMTQSPDSLAVSLGERATINCKSSQSLLYSSNQKNYLAWYQQKPGQPPKLLIYWASSRESGVPDRFSGSGSGTDFTLTISSLQAEDVAVYYCQQYYNYPLTFGQGTKLEIK

SEQ ID NO: 117 - protein, 8 aa, Artificial Sequence
  antibody heavy chain CDR1
YFDFDSYE

SEQ ID NO: 118 - protein, 7 aa, Artificial Sequence
  antibody heavy chain CDR2
IYHSGST

SEQ ID NO: 119 - protein, 10 aa, Artificial Sequence
  antibody heavy chain CDR3
ARVNMDRFDY

SEQ ID NO: 120 - protein, 117 aa, Artificial Sequence
  antibody heavy chain variable region
QVQLVQSGGGLVQPGGSLRLSCAASYFDFDSYEMSWVRQAPGKGLEWIGSIYHSGSTYYNPSLKSRVTISRDNSKNTLYLQMNTLRAEDTATYYCARVNMDRFDYWGQGTLVTVSSS

SEQ ID NO: 121 - protein, 8 aa, Artificial Sequence
  antibody heavy chain CDR1
GFTFSSYA

SEQ ID NO: 122 - protein, 8 aa, Artificial Sequence
  antibody heavy chain CDR2
IQKQGLPT

SEQ ID NO: 123 - protein, 9 aa, Artificial Sequence
  antibody heavy chain CDR3
AKNRAKFDY

SEQ ID NO: 124 - protein, 6 aa, Artificial Sequence
  antibody light chain CDR1
QSISSY

SEQ ID NO: 125 - protein, 9 aa, Artificial Sequence
  antibody light chain CDR3
QQNRGFPLT

SEQ ID NO: 126 - protein, 116 aa, ArtificiaL Sequence
  antibody heavy chain variable region
EVQLLESGGGLVQPGGSLRLSCAASGFTFSSYAMSWVRQAPGKGLEWVSTIQKQGLPTQYADSVKGRFTISRDNSKNTLYLQMNSLRAEDTAVYYCAKNRAKFDYWGQGTLVTVSS

SEQ ID NO: 127 - protein, 107 aa, Artificial Sequence
  antibody light chain variable region
DIQMTQSPSSLSASVGDRVTITCRASQSISSYLNWYQQKPGKAPKLLIYNASMLQSGVPSRFSGSGSGTDFTLTISSLQPEDFATYYCQQNRGFPLTFGQGTKVEIK

SEQ ID NO: 128 - protein, 107 aa, Artificial Sequence
  antibody light chain variable region
DIQMTQSPSSLSASVGDRVTITCRASQSISSYLNWYQQKPGKAPKLLIYAASMLQSGVPSRFSGSGSGTDFTLTISSLQPEDFATYYCQQNRGFPLTFGQGTKVEIK

SEQ ID NO: 129 - protein, 4 aa, Artificial Sequence
  cleavage site
IEGR

SEQ ID NO: 130 - protein, 10 aa, Artificial Sequence
  fusion peptide
SAWSHPQFEK

SEQ ID NO: 131 - protein, 29 aa, Artificial Sequence
  fusion peptide
GGSGGAPEQKLISEEDLGSHHHHHHHHHH

SEQ ID NO: 132 - DNA, 30 bp, Artificial Sequence
  affinity tag
caccaccatcatcaccaccatcaccatcac

SEQ ID NO: 133 - DNA, 30 bp, Artificial Sequence
  affinity tag
gagcagaagctgatctctgaggaagatctg

SEQ ID NO: 134 - DNA, 45 bp, Artificial Sequence
  linker
ggaggcggaggatctggtggtggtggatctggcggcggaggtagc

SEQ ID NO: 135 - DNA, 15 bp, Artificial Sequence
  linker
ggaggcggaggatct

SEQ ID NO: 136 - DNA, 60 bp, Artificial Sequence
  linker
ggaggcggaggatctggtggtggtggatctggcggcggaggtagcggaggcggaggatct

SEQ ID NO: 137 - DNA, 75 bp, Artificial Sequence
  linker
ggaggcggaggatctggtggtggtggatctggcggcggaggtagcggaggcggaggatctggtggtggtggatct

SEQ ID NO: 138 - DNA, 21 bp, Artificial Sequence
  linker
ggaggttctggcggtgctcct

SEQ ID NO: 139 - DNA, 45 bp, Artificial Sequence
  linker
ggctctcctacacctactcctaccacacctacaccaacacctaca

SEQ ID NO: 140 - DNA, 51 bp, Artificial Sequence
  linker
gccgaggccgctgccaaggaggccgctgcaaaggaggccgctgcaaaggcc

SEQ ID NO: 141 - DNA, 153 bp, Artificial Sequence
  multimerization domain
ggagttagactgtgggctaccagacaggctatgctcggacaggtgcacgaagtgcctgaaggctggctgatcttcgtggccgagcaagaggaactgtacgtgcgggtgcagaacggcttcagaaaggtgcagctggaagctagaacccctctc

SEQ ID NO: 142 - DNA, 117 bp, Artificial Sequence
  multimerization domain
aaacctctggatggcgagtacttcacactgcagatccggggcagagaacgcttcgagatgttcagagagctgaacgaggccctggaactgaaggatgcccaggccggaaaagaaccc

SEQ ID NO: 143 - DNA, 99 bp, Artificial Sequence
  multimerization domain
agactgaagcagatcgaggacaagctggaagagatcctgtccaagctgtaccacatcgagaacgagctggcccggatcaagaagctgctgggcgaacga

SEQ ID NO: 144 - DNA, 732 bp, Artificial Sequence
  fusion protein
caggactccacctctgatctgatccctgctcctccactgtccaaggtgccactgcagcagaacttccaggacaaccagttccacggcaagtggtacgtcgtcggccaggccggaaacatcagactgagagaggacaaggaccccatcaagatgatggctaccatctacgagctgaaagaggataagtcctacgacgtcaccatggtcaagttcgacgacaaaaagtgtatgtacgacatctggaccttcgtgcccggctctcagcctggcgagtttaccctgggcaagatcaagagcttccccggccacacctcttctctcgtgcgtgtggtgtccaccaactacaaccagcacgccatggtgttcttcaagttcgtgttccagaaccgggaagagttctacatcaccctgtacggccggaccaaagagctgacctccgaactgaaagagaacttcatccggttctccaagagcctgggcctgcctgagaaccacatcgtgttccctgtgcctatcgaccagtgcatcgatggcggaggcggaggatctggtggtggtggatctggcggcggaggtagcggagttagactgtgggctaccagacaggctatgctcggacaggtgcacgaagtgcctgaaggctggctgatcttcgtggccgagcaagaggaactgtacgtgcgggtgcagaacggcttcagaaaggtgcagctggaagctagaacccctctc

SEQ ID NO: 145 - DNA, 702 bp, Artificial Sequence
  fusion protein
caggactccacctctgatctgatccctgctcctccactgtccaaggtgccactgcagcagaacttccaggacaaccagttccacggcaagtggtacgtcgtcggccaggccggaaacatcagactgagagaggacaaggaccccatcaagatgatggctaccatctacgagctgaaagaggataagtcctacgacgtcaccatggtcaagttcgacgacaaaaagtgtatgtacgacatctggaccttcgtgcccggctctcagcctggcgagtttaccctgggcaagatcaagagcttccccggccacacctcttctctcgtgcgtgtggtgtccaccaactacaaccagcacgccatggtgttcttcaagttcgtgttccagaaccgggaagagttctacatcaccctgtacggccggaccaaagagctgacctccgaactgaaagagaacttcatccggttctccaagagcctgggcctgcctgagaaccacatcgtgttccctgtgcctatcgaccagtgcatcgatggcggaggcggaggatctggagttagactgtgggctaccagacaggctatgctcggacaggtgcacgaagtgcctgaaggctggctgatcttcgtggccgagcaagaggaactgtacgtgcgggtgcagaacggcttcagaaaggtgcagctggaagctagaacccctctc

SEQ ID NO: 146 - DNA, 762 bp, Artificial Sequence
  fusion protein
caggactccacctctgatctgatccctgctcctccactgtccaaggtgccactgcagcagaacttccaggacaaccagttccacggcaagtggtacgtcgtcggccaggccggaaacatcagactgagagaggacaaggaccccatcaagatgatggctaccatctacgagctgaaagaggataagtcctacgacgtcaccatggtcaagttcgacgacaaaaagtgtatgtacgacatctggaccttcgtgcccggctctcagcctggcgagtttaccctgggcaagatcaagagcttccccggccacacctcttctctcgtgcgtgtggtgtccaccaactacaaccagcacgccatggtgttcttcaagttcgtgttccagaaccgggaagagttctacatcaccctgtacggccggaccaaagagctgacctccgaactgaaagagaacttcatccggttctccaagagcctgggcctgcctgagaaccacatcgtgttccctgtgcctatcgaccagtgcatcgatggcggaggcggaggatctggtggtggtggatctggcggcggaggtagcggaggcggaggatctggtggtggtggatctggagttagactgtgggctaccagacaggctatgctcggacaggtgcacgaagtgcctgaaggctggctgatcttcgtggccgagcaagaggaactgtacgtgcgggtgcagaacggcttcagaaaggtgcagctggaagctagaacccctctc

SEQ ID NO: 147 - DNA, 732 bp, Artificial Sequence
  fusion protein
caggactccacctctgatctgatccctgctcctccactgtccaaggtgccactgcagcagaacttccaggacaaccagttccacggcaagtggtacgtcgtcggccaggccggaaacatcagactgagagaggacaaggaccccatcaagatgatggctaccatctacgagctgaaagaggataagtcctacgacgtcaccatggtcaagttcgacgacaaaaagtgtatgtacgacatctggaccttcgtgcccggctctcagcctggcgagtttaccctgggcaagatcaagagcttccccggccacacctcttctctcgtgcgtgtggtgtccaccaactacaaccagcacgccatggtgttcttcaagttcgtgttccagaaccgggaagagttctacatcaccctgtacggccggaccaaagagctgacctccgaactgaaagagaacttcatccggttctccaagagcctgggcctgcctgagaaccacatcgtgttccctgtgcctatcgaccagtgcatcgatggcggctctcctacacctactcctaccacacctacaccaacacctacaggagttagactgtgggctaccagacaggctatgctcggacaggtgcacgaagtgcctgaaggctggctgatcttcgtggccgagcaagaggaactgtacgtgcgggtgcagaacggcttcagaaaggtgcagctggaagctagaacccctctc

SEQ ID NO: 148 - DNA, 738 bp, Artificial Sequence
  fusion protein
caggactccacctctgatctgatccctgctcctccactgtccaaggtgccactgcagcagaacttccaggacaaccagttccacggcaagtggtacgtcgtcggccaggccggaaacatcagactgagagaggacaaggaccccatcaagatgatggctaccatctacgagctgaaagaggataagtcctacgacgtcaccatggtcaagttcgacgacaaaaagtgtatgtacgacatctggaccttcgtgcccggctctcagcctggcgagtttaccctgggcaagatcaagagcttccccggccacacctcttctctcgtgcgtgtggtgtccaccaactacaaccagcacgccatggtgttcttcaagttcgtgttccagaaccgggaagagttctacatcaccctgtacggccggaccaaagagctgacctccgaactgaaagagaacttcatccggttctccaagagcctgggcctgcctgagaaccacatcgtgttccctgtgcctatcgaccagtgcatcgatggcgccgaggccgctgccaaggaggccgctgcaaaggaggccgctgcaaaggccggagttagactgtgggctaccagacaggctatgctcggacaggtgcacgaagtgcctgaaggctggctgatcttcgtggccgagcaagaggaactgtacgtgcgggtgcagaacggcttcagaaaggtgcagctggaagctagaacccctctc

SEQ ID NO: 149 - DNA, 732 bp, Artificial Sequence
  fusion protein
ggagttagactgtgggctaccagacaggctatgctcggacaggtgcacgaagtgcctgaaggctggctgatcttcgtggccgagcaagaggaactgtacgtgcgggtgcagaacggcttcagaaaggtgcagctggaagctagaacccctctcggaggcggaggatctggtggtggtggatctggcggcggaggtagccaggactccacctctgatctgatccctgctcctccactgtccaaggtgccactgcagcagaacttccaggacaaccagttccacggcaagtggtacgtcgtcggccaggccggaaacatcagactgagagaggacaaggaccccatcaagatgatggctaccatctacgagctgaaagaggataagtcctacgacgtcaccatggtcaagttcgacgacaaaaagtgtatgtacgacatctggaccttcgtgcccggctctcagcctggcgagtttaccctgggcaagatcaagagcttccccggccacacctcttctctcgtgcgtgtggtgtccaccaactacaaccagcacgccatggtgttcttcaagttcgtgttccagaaccgggaagagttctacatcaccctgtacggccggaccaaagagctgacctccgaactgaaagagaacttcatccggttctccaagagcctgggcctgcctgagaaccacatcgtgttccctgtgcctatcgaccagtgcatcgatggc

SEQ ID NO: 150 - DNA, 702 bp, Artificial Sequence
  fusion protein
ggagttagactgtgggctaccagacaggctatgctcggacaggtgcacgaagtgcctgaaggctggctgatcttcgtggccgagcaagaggaactgtacgtgcgggtgcagaacggcttcagaaaggtgcagctggaagctagaacccctctcggaggcggaggatctcaggactccacctctgatctgatccctgctcctccactgtccaaggtgccactgcagcagaacttccaggacaaccagttccacggcaagtggtacgtcgtcggccaggccggaaacatcagactgagagaggacaaggaccccatcaagatgatggctaccatctacgagctgaaagaggataagtcctacgacgtcaccatggtcaagttcgacgacaaaaagtgtatgtacgacatctggaccttcgtgcccggctctcagcctggcgagtttaccctgggcaagatcaagagcttccccggccacacctcttctctcgtgcgtgtggtgtccaccaactacaaccagcacgccatggtgttcttcaagttcgtgttccagaaccgggaagagttctacatcaccctgtacggccggaccaaagagctgacctccgaactgaaagagaacttcatccggttctccaagagcctgggcctgcctgagaaccacatcgtgttccctgtgcctatcgaccagtgcatcgatggc

SEQ ID NO: 151 - DNA, 762 bp, Artificial Sequence
  fusion protein
ggagttagactgtgggctaccagacaggctatgctcggacaggtgcacgaagtgcctgaaggctggctgatcttcgtggccgagcaagaggaactgtacgtgcgggtgcagaacggcttcagaaaggtgcagctggaagctagaacccctctcggaggcggaggatctggtggtggtggatctggcggcggaggtagcggaggcggaggatctggtggtggtggatctcaggactccacctctgatctgatccctgctcctccactgtccaaggtgccactgcagcagaacttccaggacaaccagttccacggcaagtggtacgtcgtcggccaggccggaaacatcagactgagagaggacaaggaccccatcaagatgatggctaccatctacgagctgaaagaggataagtcctacgacgtcaccatggtcaagttcgacgacaaaaagtgtatgtacgacatctggaccttcgtgcccggctctcagcctggcgagtttaccctgggcaagatcaagagcttccccggccacacctcttctctcgtgcgtgtggtgtccaccaactacaaccagcacgccatggtgttcttcaagttcgtgttccagaaccgggaagagttctacatcaccctgtacggccggaccaaagagctgacctccgaactgaaagagaacttcatccggttctccaagagcctgggcctgcctgagaaccacatcgtgttccctgtgcctatcgaccagtgcatcgatggc

SEQ ID NO: 152 - DNA, 678 bp, Artificial Sequence
  fusion protein
caggactccacctctgatctgatccctgctcctccactgtccaaggtgccactgcagcagaacttccaggacaaccagttccacggcaagtggtacgtcgtcggccaggccggaaacatcagactgagagaggacaaggaccccatcaagatgatggctaccatctacgagctgaaagaggataagtcctacgacgtcaccatggtcaagttcgacgacaaaaagtgtatgtacgacatctggaccttcgtgcccggctctcagcctggcgagtttaccctgggcaagatcaagagcttccccggccacacctcttctctcgtgcgtgtggtgtccaccaactacaaccagcacgccatggtgttcttcaagttcgtgttccagaaccgggaagagttctacatcaccctgtacggccggaccaaagagctgacctccgaactgaaagagaacttcatccggttctccaagagcctgggcctgcctgagaaccacatcgtgttccctgtgcctatcgaccagtgcatcgatggcggaggcggaggatctggtggtggtggatctggcggcggaggtagcagactgaagcagatcgaggacaagctggaagagatcctgtccaagctgtaccacatcgagaacgagctggcccggatcaagaagctgctgggcgaacga

SEQ ID NO: 153 - DNA, 696 bp, Artificial Sequence
  fusion protein
caggactccacctctgatctgatccctgctcctccactgtccaaggtgccactgcagcagaacttccaggacaaccagttccacggcaagtggtacgtcgtcggccaggccggaaacatcagactgagagaggacaaggaccccatcaagatgatggctaccatctacgagctgaaagaggataagtcctacgacgtcaccatggtcaagttcgacgacaaaaagtgtatgtacgacatctggaccttcgtgcccggctctcagcctggcgagtttaccctgggcaagatcaagagcttccccggccacacctcttctctcgtgcgtgtggtgtccaccaactacaaccagcacgccatggtgttcttcaagttcgtgttccagaaccgggaagagttctacatcaccctgtacggccggaccaaagagctgacctccgaactgaaagagaacttcatccggttctccaagagcctgggcctgcctgagaaccacatcgtgttccctgtgcctatcgaccagtgcatcgatggcggaggcggaggatctggtggtggtggatctggcggcggaggtagcaaacctctggatggcgagtacttcacactgcagatccggggcagagaacgcttcgagatgttcagagagctgaacgaggccctggaactgaaggatgcccaggccggaaaagaaccc

SEQ ID NO: 154 - DNA, 1311 bp, Artificial Sequence
  fusion protein
caggactccacctctgatctgatccctgctcctccactgtccaaggtgccactgcagcagaacttccaggacaaccagttccacggcaagtggtacgtcgtcggccaggccggaaacatcagactgagagaggacaaggaccccatcaagatgatggctaccatctacgagctgaaagaggataagtcctacgacgtcaccatggtcaagttcgacgacaaaaagtgtatgtacgacatctggaccttcgtgcccggctctcagcctggcgagtttaccctgggcaagatcaagagcttccccggccacacctcttctctcgtgcgtgtggtgtccaccaactacaaccagcacgccatggtgttcttcaagttcgtgttccagaaccgggaagagttctacatcaccctgtacggccggaccaaagagctgacctccgaactgaaagagaacttcatccggttctccaagagcctgggcctgcctgagaaccacatcgtgttccctgtgcctatcgaccagtgcatcgatggcggaggcggaggatctggtggtggtggatctggcggcggaggtagcggagttagactgtgggctaccagacaggctatgctcggacaggtgcacgaagtgcctgaaggctggctgatcttcgtggccgagcaagaggaactgtacgtgcgggtgcagaacggcttcagaaaggtgcagctggaagctagaacccctctcggaggcggaggatctggtggtggtggatctggcggcggaggtagccaggactccacctctgatctgatccctgctcctccactgtccaaggtgccactgcagcagaacttccaggacaaccagttccacggcaagtggtacgtcgtcggccaggccggaaacatcagactgagagaggacaaggaccccatcaagatgatggctaccatctacgagctgaaagaggataagtcctacgacgtcaccatggtcaagttcgacgacaaaaagtgtatgtacgacatctggaccttcgtgcccggctctcagcctggcgagtttaccctgggcaagatcaagagcttccccggccacacctcttctctcgtgcgtgtggtgtccaccaactacaaccagcacgccatggtgttcttcaagttcgtgttccagaaccgggaagagttctacatcaccctgtacggccggaccaaagagctgacctccgaactgaaagagaacttcatccggttctccaagagcctgggcctgcctgagaaccacatcgtgttccctgtgcctatcgaccagtgcatcgatggc

SEQ ID NO: 155 - DNA, 1281 bp, Artificial Sequence
  fusion protein
caggactccacctctgatctgatccctgctcctccactgtccaaggtgccactgcagcagaacttccaggacaaccagttccacggcaagtggtacgtcgtcggccaggccggaaacatcagactgagagaggacaaggaccccatcaagatgatggctaccatctacgagctgaaagaggataagtcctacgacgtcaccatggtcaagttcgacgacaaaaagtgtatgtacgacatctggaccttcgtgcccggctctcagcctggcgagtttaccctgggcaagatcaagagcttccccggccacacctcttctctcgtgcgtgtggtgtccaccaactacaaccagcacgccatggtgttcttcaagttcgtgttccagaaccgggaagagttctacatcaccctgtacggccggaccaaagagctgacctccgaactgaaagagaacttcatccggttctccaagagcctgggcctgcctgagaaccacatcgtgttccctgtgcctatcgaccagtgcatcgatggcggaggcggaggatctggagttagactgtgggctaccagacaggctatgctcggacaggtgcacgaagtgcctgaaggctggctgatcttcgtggccgagcaagaggaactgtacgtgcgggtgcagaacggcttcagaaaggtgcagctggaagctagaacccctctcggaggcggaggatctggtggtggtggatctggcggcggaggtagccaggactccacctctgatctgatccctgctcctccactgtccaaggtgccactgcagcagaacttccaggacaaccagttccacggcaagtggtacgtcgtcggccaggccggaaacatcagactgagagaggacaaggaccccatcaagatgatggctaccatctacgagctgaaagaggataagtcctacgacgtcaccatggtcaagttcgacgacaaaaagtgtatgtacgacatctggaccttcgtgcccggctctcagcctggcgagtttaccctgggcaagatcaagagcttccccggccacacctcttctctcgtgcgtgtggtgtccaccaactacaaccagcacgccatggtgttcttcaagttcgtgttccagaaccgggaagagttctacatcaccctgtacggccggaccaaagagctgacctccgaactgaaagagaacttcatccggttctccaagagcctgggcctgcctgagaaccacatcgtgttccctgtgcctatcgaccagtgcatcgatggc

SEQ ID NO: 156 - DNA, 1341 bp, Artificial Sequence
  fusion protein
caggactccacctctgatctgatccctgctcctccactgtccaaggtgccactgcagcagaacttccaggacaaccagttccacggcaagtggtacgtcgtcggccaggccggaaacatcagactgagagaggacaaggaccccatcaagatgatggctaccatctacgagctgaaagaggataagtcctacgacgtcaccatggtcaagttcgacgacaaaaagtgtatgtacgacatctggaccttcgtgcccggctctcagcctggcgagtttaccctgggcaagatcaagagcttccccggccacacctcttctctcgtgcgtgtggtgtccaccaactacaaccagcacgccatggtgttcttcaagttcgtgttccagaaccgggaagagttctacatcaccctgtacggccggaccaaagagctgacctccgaactgaaagagaacttcatccggttctccaagagcctgggcctgcctgagaaccacatcgtgttccctgtgcctatcgaccagtgcatcgatggcggaggcggaggatctggtggtggtggatctggcggcggaggtagcggaggcggaggatctggtggtggtggatctggagttagactgtgggctaccagacaggctatgctcggacaggtgcacgaagtgcctgaaggctggctgatcttcgtggccgagcaagaggaactgtacgtgcgggtgcagaacggcttcagaaaggtgcagctggaagctagaacccctctcggaggcggaggatctggtggtggtggatctggcggcggaggtagccaggactccacctctgatctgatccctgctcctccactgtccaaggtgccactgcagcagaacttccaggacaaccagttccacggcaagtggtacgtcgtcggccaggccggaaacatcagactgagagaggacaaggaccccatcaagatgatggctaccatctacgagctgaaagaggataagtcctacgacgtcaccatggtcaagttcgacgacaaaaagtgtatgtacgacatctggaccttcgtgcccggctctcagcctggcgagtttaccctgggcaagatcaagagcttccccggccacacctcttctctcgtgcgtgtggtgtccaccaactacaaccagcacgccatggtgttcttcaagttcgtgttccagaaccgggaagagttctacatcaccctgtacggccggaccaaagagctgacctccgaactgaaagagaacttcatccggttctccaagagcctgggcctgcctgagaaccacatcgtgttccctgtgcctatcgaccagtgcatcgatggc

SEQ ID NO: 157 - DNA, 1317 bp, Artificial Sequence
  fusion protein
caggactccacctctgatctgatccctgctcctccactgtccaaggtgccactgcagcagaacttccaggacaaccagttccacggcaagtggtacgtcgtcggccaggccggaaacatcagactgagagaggacaaggaccccatcaagatgatggctaccatctacgagctgaaagaggataagtcctacgacgtcaccatggtcaagttcgacgacaaaaagtgtatgtacgacatctggaccttcgtgcccggctctcagcctggcgagtttaccctgggcaagatcaagagcttccccggccacacctcttctctcgtgcgtgtggtgtccaccaactacaaccagcacgccatggtgttcttcaagttcgtgttccagaaccgggaagagttctacatcaccctgtacggccggaccaaagagctgacctccgaactgaaagagaacttcatccggttctccaagagcctgggcctgcctgagaaccacatcgtgttccctgtgcctatcgaccagtgcatcgatggcgccgaggccgctgccaaggaggccgctgcaaaggaggccgctgcaaaggccggagttagactgtgggctaccagacaggctatgctcggacaggtgcacgaagtgcctgaaggctggctgatcttcgtggccgagcaagaggaactgtacgtgcgggtgcagaacggcttcagaaaggtgcagctggaagctagaacccctctcggaggcggaggatctggtggtggtggatctggcggcggaggtagccaggactccacctctgatctgatccctgctcctccactgtccaaggtgccactgcagcagaacttccaggacaaccagttccacggcaagtggtacgtcgtcggccaggccggaaacatcagactgagagaggacaaggaccccatcaagatgatggctaccatctacgagctgaaagaggataagtcctacgacgtcaccatggtcaagttcgacgacaaaaagtgtatgtacgacatctggaccttcgtgcccggctctcagcctggcgagtttaccctgggcaagatcaagagcttccccggccacacctcttctctcgtgcgtgtggtgtccaccaactacaaccagcacgccatggtgttcttcaagttcgtgttccagaaccgggaagagttctacatcaccctgtacggccggaccaaagagctgacctccgaactgaaagagaacttcatccggttctccaagagcctgggcctgcctgagaaccacatcgtgttccctgtgcctatcgaccagtgcatcgatggc

SEQ ID NO: 158 - DNA, 1311 bp, Artificial Sequence
  fusion protein
caggactccacctctgatctgatccctgctcctccactgtccaaggtgccactgcagcagaacttccaggacaaccagttccacggcaagtggtacgtcgtcggccaggccggaaacatcagactgagagaggacaaggaccccatcaagatgatggctaccatctacgagctgaaagaggataagtcctacgacgtcaccatggtcaagttcgacgacaaaaagtgtatgtacgacatctggaccttcgtgcccggctctcagcctggcgagtttaccctgggcaagatcaagagcttccccggccacacctcttctctcgtgcgtgtggtgtccaccaactacaaccagcacgccatggtgttcttcaagttcgtgttccagaaccgggaagagttctacatcaccctgtacggccggaccaaagagctgacctccgaactgaaagagaacttcatccggttctccaagagcctgggcctgcctgagaaccacatcgtgttccctgtgcctatcgaccagtgcatcgatggcggaggcggaggatctggtggtggtggatctggcggcggaggtagccaggactccacctctgatctgatccctgctcctccactgtccaaggtgccactgcagcagaacttccaggacaaccagttccacggcaagtggtacgtcgtcggccaggccggaaacatcagactgagagaggacaaggaccccatcaagatgatggctaccatctacgagctgaaagaggataagtcctacgacgtcaccatggtcaagttcgacgacaaaaagtgtatgtacgacatctggaccttcgtgcccggctctcagcctggcgagtttaccctgggcaagatcaagagcttccccggccacacctcttctctcgtgcgtgtggtgtccaccaactacaaccagcacgccatggtgttcttcaagttcgtgttccagaaccgggaagagttctacatcaccctgtacggccggaccaaagagctgacctccgaactgaaagagaacttcatccggttctccaagagcctgggcctgcctgagaaccacatcgtgttccctgtgcctatcgaccagtgcatcgatggcggaggcggaggatctggtggtggtggatctggcggcggaggtagcggagttagactgtgggctaccagacaggctatgctcggacaggtgcacgaagtgcctgaaggctggctgatcttcgtggccgagcaagaggaactgtacgtgcgggtgcagaacggcttcagaaaggtgcagctggaagctagaacccctctc

SEQ ID NO: 159 - DNA, 1311 bp, Artificial Sequence
  fusion protein
ggagttagactgtgggctaccagacaggctatgctcggacaggtgcacgaagtgcctgaaggctggctgatcttcgtggccgagcaagaggaactgtacgtgcgggtgcagaacggcttcagaaaggtgcagctggaagctagaacccctctcggaggcggaggatctggtggtggtggatctggcggcggaggtagccaggactccacctctgatctgatccctgctcctccactgtccaaggtgccactgcagcagaacttccaggacaaccagttccacggcaagtggtacgtcgtcggccaggccggaaacatcagactgagagaggacaaggaccccatcaagatgatggctaccatctacgagctgaaagaggataagtcctacgacgtcaccatggtcaagttcgacgacaaaaagtgtatgtacgacatctggaccttcgtgcccggctctcagcctggcgagtttaccctgggcaagatcaagagcttccccggccacacctcttctctcgtgcgtgtggtgtccaccaactacaaccagcacgccatggtgttcttcaagttcgtgttccagaaccgggaagagttctacatcaccctgtacggccggaccaaagagctgacctccgaactgaaagagaacttcatccggttctccaagagcctgggcctgcctgagaaccacatcgtgttccctgtgcctatcgaccagtgcatcgatggcggaggcggaggatctggtggtggtggatctggcggcggaggtagccaggactccacctctgatctgatccctgctcctccactgtccaaggtgccactgcagcagaacttccaggacaaccagttccacggcaagtggtacgtcgtcggccaggccggaaacatcagactgagagaggacaaggaccccatcaagatgatggctaccatctacgagctgaaagaggataagtcctacgacgtcaccatggtcaagttcgacgacaaaaagtgtatgtacgacatctggaccttcgtgcccggctctcagcctggcgagtttaccctgggcaagatcaagagcttccccggccacacctcttctctcgtgcgtgtggtgtccaccaactacaaccagcacgccatggtgttcttcaagttcgtgttccagaaccgggaagagttctacatcaccctgtacggccggaccaaagagctgacctccgaactgaaagagaacttcatccggttctccaagagcctgggcctgcctgagaaccacatcgtgttccctgtgcctatcgaccagtgcatcgatggc

SEQ ID NO: 160 - DNA, 1521 bp, Artificial Sequence
  fusion protein
caggttcagctggtacagtctggcgctgaagtgaagaaacctggggctagtgtgaaagtcagctgtaaggctagcgggtacacctttaccgactacgagatgcactgggttaggcaagcacctggacaaggcctggaatggatgggtgctcttgaccccaaaaccggagatacagcctattcccagaagttcaagggcagagtcactctgacagccgacaagtccacatctaccgcctatatggagctcagctcactgacgtcagaggatacagccgtgtactactgcactcggttctattcctacacctattggggacagggcactctcgtgactctcgtgtctagtggaggcggaggatctggtggtggtggatctggcggcggaggtagcggaggcggaggatctgatgtcgtgatgacacagagtcctctctctctgccagtgacacctggagaacctgccagcattagctgcagaagttcccagtcccttgtgcactctaaccgcaatacctatctgcactggtatctgcagaaacccggacagtcaccccaactgctgatctacaaggtgtctaaccggttttccggagttcccgatcgattcagcggttcaggctctggtacagacttcaccctcaagatctccagggtcgaagctgaggacgtaggcgtgtactactgtagccagaatacgcatgtcccacccacttttgggcaaggcaccaaactggagataaagggaggcggaggatctggtggtggtggatctggcggcggaggtagcggagttagactgtgggctaccagacaggctatgctcggacaggtgcacgaagtgcctgaaggctggctgatcttcgtggccgagcaagaggaactgtacgtgcgggtgcagaacggcttcagaaaggtgcagctggaagctagaacccctctcggaggcggaggatctggtggtggtggatctggcggcggaggtagccaggactccacctctgatctgatccctgctcctccactgtccaaggtgccactgcagcagaacttccaggacaaccagttccacggcaagtggtacgtcgtcggccaggccggaaacatcagactgagagaggacaaggaccccatcaagatgatggctaccatctacgagctgaaagaggataagtcctacgacgtcaccatggtcaagttcgacgacaaaaagtgtatgtacgacatctggaccttcgtgcccggctctcagcctggcgagtttaccctgggcaagatcaagagcttccccggccacacctcttctctcgtgcgtgtggtgtccaccaactacaaccagcacgccatggtgttcttcaagttcgtgttccagaaccgggaagagttctacatcaccctgtacggccggaccaaagagctgacctccgaactgaaagagaacttcatccggttctccaagagcctgggcctgcctgagaaccacatcgtgttccctgtgcctatcgaccagtgcatcgatggc

SEQ ID NO: 161 - DNA, 1311 bp, Artificial Sequence
  fusion protein
caggactccacctccgatctgattccagctcctccactgtccaaggtgcccctgcagcagaacttccaggacaaccagttccaaggcaagtggtacgtcgtgggcagagctggcaatgtgggcctgagagaggacaaggaccctcctaagatgtgggccaccatctacgagctgaaagaggataagtcctacgacgtgaccaacgttcgcttcgcccggaagaagtgcacctactccatcggcaccttcgtgcctggatctcagcctggcgagtttaccctgggccagatcaagtctgagcctggcggcacagccaacctcgtcagagtggtgtccaccaactacaaccagcacgccatggtgttcttcaaagaggtgtaccagaaccgcgagatcttcttcatcatcctgtacggccggaccaaagagctgacctccgaactgaaagagaacttcatccggttctccaagagcctgggcctgcctgagaaccacatcgtgttccctgtgcctatcgaccagtgcatcgatggcggaggcggaggatctggtggtggtggatctggcggcggaggtagcggagttagactgtgggctaccagacaggctatgctcggacaggtgcacgaagtgcctgaaggctggctgatcttcgtggccgagcaagaggaactgtacgtgcgggtgcagaacggcttcagaaaggtgcagctggaagctagaacccctctcggaggcggaggatctggtggtggtggatctggcggcggaggtagccaggactccacctctgatctgatccctgctcctccactgtccaaggtgccactgcagcagaacttccaggacaaccagttccacggcaagtggtacgtcgtcggccaggccggaaacatcagactgagagaggacaaggaccccatcaagatgatggctaccatctacgagctgaaagaggataagtcctacgacgtcaccatggtcaagttcgacgacaaaaagtgtatgtacgacatctggaccttcgtgcccggctctcagcctggcgagtttaccctgggcaagatcaagagcttccccggccacacctcttctctcgtgcgtgtggtgtccaccaactacaaccagcacgccatggtgttcttcaagttcgtgttccagaaccgggaagagttctacatcaccctgtacggccggaccaaagagctgacctccgaactgaaagagaacttcatccggttctccaagagcctgggcctgcctgagaaccacatcgtgttccctgtgcctatcgaccagtgcatcgatggc

SEQ ID NO: 162 - DNA, 744 bp, Artificial Sequence
  antibody single chain Fv fragment
caggttcagctggtacagtctggcgctgaagtgaagaaacctggggctagtgtgaaagtcagctgtaaggctagcgggtacacctttaccgactacgagatgcactgggttaggcaagcacctggacaaggcctggaatggatgggtgctcttgaccccaaaaccggagatacagcctattcccagaagttcaagggcagagtcactctgacagccgacaagtccacatctaccgcctatatggagctcagctcactgacgtcagaggatacagccgtgtactactgcactcggttctattcctacacctattggggacagggcactctcgtgactctcgtgtctagtggaggcggaggatctggtggtggtggatctggcggcggaggtagcggaggcggaggatctgatgtcgtgatgacacagagtcctctctctctgccagtgacacctggagaacctgccagcattagctgcagaagttcccagtcccttgtgcactctaaccgcaatacctatctgcactggtatctgcagaaacccggacagtcaccccaactgctgatctacaaggtgtctaaccggttttccggagttcccgatcgattcagcggttcaggctctggtacagacttcaccctcaagatctccagggtcgaagctgaggacgtaggcgtgtactactgtagccagaatacgcatgtcccacccacttttgggcaaggcaccaaactggagataaag

SEQ ID NO: 163 - DNA, 87 bp, Artificial Sequence
  fusion peptide
ggaggttctggcggtgctcctgagcagaagctgatctctgaggaagatctgggctcccaccaccatcatcaccaccatcaccatcac

SEQ ID NO: 164 - protein, 437 aa, Artificial Sequence
  fusion protein
QDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDGGGGGSGGGGSGGGGSGVRLWATRQAMLGQVHEVPEGWLIFVAEQEELYVRVQNGFRKVQLEARTPLGGGGSGGGGSGGGGSQDSTSDLIPAPPLSKVPLQQNFQDSQFQGKWYVVGFAGNYRLREDKDPGKMQATIYELREDKAYQVTGVLFPHKKCHDDIVIFVPGSQPGEFILGWIKSDPGITSDLVRVVSTDYNQHAMVFFKWVFQNREWFYITLYGRTKALTSELKENFIRFSKSLGLPEDHIVFPVPIDQCIDG

SEQ ID NO: 165 - protein, 437 aa, Artificial Sequence
  fusion protein
QDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDGGGGGSGGGGSGGGGSQDSTSDLIPAPPLSKVPLQQNFQDSQFQGKWYVVGFAGNYRLREDKDPGKMQATIYELREDKAYQVTGVLFPHKKCHDDIVIFVPGSQPGEFILGWIKSDPGITSDLVRVVSTDYNQHAMVFFKWVFQNREWFYITLYGRTKALTSELKENFIRFSKSLGLPEDHIVFPVPIDQCIDGGGGGSGGGGSGGGGSGVRLWATRQAMLGQVHEVPEGWLIFVAEQEELYVRVQNGFRKVQLEARTPL

SEQ ID NO: 166 - protein, 437 aa, Artificial Sequence
  fusion protein
QDSTSDLIPAPPLSKVPLQQNFQDSQFQGKWYVVGFAGNYRLREDKDPGKMQATIYELREDKAYQVTGVLFPHKKCHDDIVIFVPGSQPGEFILGWIKSDPGITSDLVRVVSTDYNQHAMVFFKWVFQNREWFYITLYGRTKALTSELKENFIRFSKSLGLPEDHIVFPVPIDQCIDGGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDGGGGGSGGGGSGGGGSGVRLWATRQAMLGQVHEVPEGWLIFVAEQEELYVRVQNGFRKVQLEARTPL

SEQ ID NO: 167 - protein, 506 aa, Artificial Sequence
  fusion protein
QVQLQESGPGLVKPSETLSITCTVSGFSLSNYDISWIRQPPGKGLEWLGVIWTGGATNYNPALKSRLTISRDNSKNQVSLKMSSVTAADTAVYYCVRDSNYRYDEPFTYWGQGTLVTVSSGGGGSGGGGSGGGGSGGGGSEIVLTQSPDTLSVTPKEKVTLTCRASQSIGTNIHWFQQKPGQSPKLLIKYASESISGVPSRFSGSGSGTDFTLTINSVEAEDAATYYCQQSNSWPYTFGQGTKLEIKGGGGSGGGGSGGGGSGVRLWATRQAMLGQVHEVPEGWLIFVAEQEELYVRVQNGFRKVQLEARTPLGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 168 - DNA, 1311 bp, Artificial Sequence
  fusion protein
caggactccacctctgatctgatccctgctcctccactgtccaaggtgccactgcagcagaacttccaggacaaccagttccacggcaagtggtacgtcgtcggccaggccggaaacatcagactgagagaggacaaggaccccatcaagatgatggctaccatctacgagctgaaagaggataagtcctacgacgtcaccatggtcaagttcgacgacaaaaagtgtatgtacgacatctggaccttcgtgcccggctctcagcctggcgagtttaccctgggcaagatcaagagcttccccggccacacctcttctctcgtgcgtgtggtgtccaccaactacaaccagcacgccatggtgttcttcaagttcgtgttccagaaccgggaagagttctacatcaccctgtacggccggaccaaagagctgacctccgaactgaaagagaacttcatccggttctccaagagcctgggcctgcctgagaaccacatcgtgttccctgtgcctatcgaccagtgcatcgatggcggaggcggaggatctggtggtggtggatctggcggcggaggtagcggagttagactgtgggctaccagacaggctatgctcggacaggtgcacgaagtgcctgaaggctggctgatcttcgtggccgagcaagaggaactgtacgtgcgggtgcagaacggcttcagaaaggtgcagctggaagctagaacccctctcggaggcggaggatctggtggtggtggatctggcggcggaggtagccaggattctacctccgatctgatccccgctcctccactgtctaaggtgccactgcagcagaacttccaggactcccagttccaaggcaagtggtacgtcgtgggcttcgccggcaactacagactgagagaggacaaggaccccggcaagatgcaggccaccatctacgagctgcgcgaggataaggcctaccaagtgaccggcgtgctgttccctcacaagaagtgccacgacgacatcgtgatcttcgtgcccggatctcagcccggcgagtttatcctcggctggatcaagtctgaccctggcatcacctccgacctcgtcagagtggtgtccaccgactacaaccagcacgccatggtgttcttcaaatgggtgttccagaaccgcgagtggttctacatcaccctgtacggcaggaccaaggctctgacctccgagctgaaagagaacttcatccggttctccaagagcctgggcctgcctgaggatcacatcgtgttccctgtgcctatcgaccagtgcatcgatggc

SEQ ID NO: 169 - DNA, 1311 bp, Artificial Sequence
  fusion protein
caggactccacctctgatctgatccctgctcctccactgtccaaggtgccactgcagcagaacttccaggacaaccagttccacggcaagtggtacgtcgtcggccaggccggaaacatcagactgagagaggacaaggaccccatcaagatgatggctaccatctacgagctgaaagaggataagtcctacgacgtcaccatggtcaagttcgacgacaaaaagtgtatgtacgacatctggaccttcgtgcccggctctcagcctggcgagtttaccctgggcaagatcaagagcttccccggccacacctcttctctcgtgcgtgtggtgtccaccaactacaaccagcacgccatggtgttcttcaagttcgtgttccagaaccgggaagagttctacatcaccctgtacggccggaccaaagagctgacctccgaactgaaagagaacttcatccggttctccaagagcctgggcctgcctgagaaccacatcgtgttccctgtgcctatcgaccagtgcatcgatggcggaggcggaggatctggtggtggtggatctggcggcggaggctctcaggattctacctccgacctgattccagcacctcctctgagcaaagtccctctgcagcaaaattttcaggactctcagtttcaaggcaagtggtatgttgtgggcttcgccggcaactaccggctgagggaagataaggatcccggcaagatgcaggccacaatctacgaactccgcgaggataaggcttaccaagtgaccggcgtgctgttccctcacaagaagtgccacgacgacatcgtgatctttgtgcctggaagccagccaggcgaattcatcctcggctggatcaagtctgaccccggcatcacatccgacctcgtcagagtggtgtctaccgattacaatcaacatgctatggtctttttcaaatgggttttccagaacagagagtggttttacatcacactctatggccgcacaaaggccctgacatccgagttgaaagaaaatttcattcgcttcagcaagtccctgggactgcccgaggatcatattgtgtttcccgtgccaattgatcagtgtattgatggcggtggtggcggtagcggtggcggtggttcaggtggcggcggttctggcgttagactgtgggctacaagacaggccatgctcggacaggtgcacgaagtgcctgaaggctggctgatcttcgtggccgagcaagaggaactgtacgtgcgggtgcagaacggcttcagaaaggtgcagctggaagctagaacccctctc

SEQ ID NO: 170 - DNA, 1311 bp, Artificial Sequence
  fusion protein
caggactccacctctgatctgatccctgctcctccactgtccaaggtgccactgcagcagaacttccaggactcccagttccaaggcaagtggtacgtcgtgggcttcgccggcaactacagactgagagaggacaaggaccccggcaagatgcaggccaccatctacgagctgcgcgaggataaggcctaccaagtgaccggcgtgctgttccctcacaagaagtgccacgacgacatcgtgatcttcgtgcccggatctcagcccggcgagtttatcctcggctggatcaagtctgaccctggcatcacctccgacctcgtcagagtggtgtccaccgactacaaccagcacgccatggtgttcttcaaatgggtgttccagaaccgcgagtggttctacatcaccctgtacggcaggaccaaggctctgacctccgagctgaaagagaacttcatccggttctccaagagcctgggcctgcctgaggatcacatcgtgttccctgtgcctatcgaccagtgcatcgatggcggaggcggaggatctggtggtggtggatctggcggcggaggttctcaggattctaccagcgacctgattcctgcacctcctctgagcaaagtccctctgcagcaaaattttcaggataaccagtttcacggaaagtggtatgttgtcggccaggccggcaatatccggctgagggaagataaggaccctatcaagatgatggctacaatctacgaactgaaagaggataagagctacgacgtcaccatggtcaagttcgacgacaaaaagtgtatgtacgacatctggacctttgtgcctggcagccagcctggcgaattcaccctgggaaagatcaagagcttccccggccacacctccagtcttgtgcgtgtggtgtctaccaattacaatcaacatgctatggtctttttcaagttcgtgtttcagaacagggaagagttttacatcacactctatggccgcaccaaagagctgaccagcgaactcaaagaaaactttatccgcttcagcaagtccctgggactgcccgagaaccatattgtgtttcccgtgccaattgatcagtgtattgatggcggtggtggcggtagcggtggcggtggaagcggcggtggcggctctggtgttagattgtgggctaccagacaggccatgctcggacaggttcacgaagtgcctgaaggatggctgatctttgtggccgagcaagaggaactgtacgtgcgggtgcagaacggcttcagaaaggtgcagctggaagctagaacccctctc

SEQ ID NO: 171 - DNA, 1518 bp, Artificial Sequence
  fusion protein
caggtccagctgcaagaatctggacctggcctggtcaagccctccgagacactgtctatcacctgtaccgtgtccggcttctccctgtccaactacgacatctcctggatcagacagcctcctggcaaaggcctggaatggctgggagtgatttggaccggcggagccaccaactacaaccccgctctgaagtcccggctgaccatctccagagacaactccaagaaccaggtgtccctgaagatgtcctccgtgaccgctgctgataccgccgtgtactactgcgtgcgggactccaactacagatacgacgagcccttcacctactggggccagggaacactggtcacagtttctagcggaggcggaggatctggtggtggtggatctggcggcggaggtagtggtggcggaggttctgaaatcgtgctgacccagtctcctgacacactgagcgtgacccctaaagaaaaagtgaccctgacctgccgggccagccagtctatcggcaccaacatccactggttccagcagaagcctggccagtctccaaagctgctgattaagtacgcctccgagtccatctccggcgtgccctctagattttctggctccggctctggcaccgacttcaccctgaccatcaactccgtggaagccgaggatgccgctacctactactgccagcagtccaactcctggccttacacctttggccagggcaccaagctggaaatcaaaggcggtggtggtagtggcggtggcggatcaggtggcggtggaagcggagttagactgtgggctaccagacaggctatgctcggacaggtgcacgaagtgcctgaaggctggctgatcttcgtggccgagcaagaggaactgtacgtgcgggtgcagaacggcttcagaaaggtgcagctggaagctagaacccctctcggaggcggaggatctggtggtggtggatctggcggcggaggtagccaggactccacctctgatctgatccctgctcctccactgtccaaggtgccactgcagcagaacttccaggacaaccagttccacggcaagtggtacgtcgtcggccaggccggaaacatcagactgagagaggacaaggaccccatcaagatgatggctaccatctacgagctgaaagaggataagtcctacgacgtcaccatggtcaagttcgacgacaaaaagtgtatgtacgacatctggaccttcgtgcccggctctcagcctggcgagtttaccctgggcaagatcaagagcttccccggccacacctcttctctcgtgcgtgtggtgtccaccaactacaaccagcacgccatggtgttcttcaagttcgtgttccagaaccgggaagagttctacatcaccctgtacggccggaccaaagagctgacctccgaactgaaagagaacttcatccggttctccaagagcctgggcctgcctgagaaccacatcgtgttccctgtgcctatcgaccagtgcatcgatggc

SEQ ID NO: 172 - protein, 247 aa, Artificial Sequence
  antibody fragment
QVQLQESGPGLVKPSETLSITCTVSGFSLSNYDISWIRQPPGKGLEWLGVIWTGGATNYNPALKSRLTISRDNSKNQVSLKMSSVTAADTAVYYCVRDSNYRYDEPFTYWGQGTLVTVSSGGGGSGGGGSGGGGSGGGGSEIVLTQSPDTLSVTPKEKVTLTCRASQSIGTNIHWFQQKPGQSPKLLIKYASESISGVPSRFSGSGSGTDFTLTINSVEAEDAATYYCQQSNSWPYTFGQGTKLEIK

SEQ ID NO: 173 - DNA, 741 bp, Artificial Sequence
  antibody fragment
caggtccagctgcaagaatctggacctggcctggtcaagccctccgagacactgtctatcacctgtaccgtgtccggcttctccctgtccaactacgacatctcctggatcagacagcctcctggcaaaggcctggaatggctgggagtgatttggaccggcggagccaccaactacaaccccgctctgaagtcccggctgaccatctccagagacaactccaagaaccaggtgtccctgaagatgtcctccgtgaccgctgctgataccgccgtgtactactgcgtgcgggactccaactacagatacgacgagcccttcacctactggggccagggaacactggtcacagtttctagcggaggcggaggatctggtggtggtggatctggcggcggaggtagtggtggcggaggttctgaaatcgtgctgacccagtctcctgacacactgagcgtgacccctaaagaaaaagtgaccctgacctgccgggccagccagtctatcggcaccaacatccactggttccagcagaagcctggccagtctccaaagctgctgattaagtacgcctccgagtccatctccggcgtgccctctagattttctggctccggctctggcaccgacttcaccctgaccatcaactccgtggaagccgaggatgccgctacctactactgccagcagtccaactcctggccttacacctttggccagggcaccaagctggaaatcaaa

SEQ ID NO: 174 - protein, 152 aa, Artificial Sequence
  lipocalin mutein
ASDEEIQDVSGTWYLKAMTVDWDCNQPIFDSVTPMTLTTLEGGNLEAKVTIFIDGKYQEVKAVLEKTDEPGKYTADGGKHVAYIIRSHVKDHYIFYSEGQCPGIPVPGVWLVGRDPKNNLEALEDFEKAAGARGLSTESILIPRQSETSSPG

SEQ ID NO: 175 - protein, 152 aa, Artificial Sequence
  lipocalin mutein
ASDEEIQDVSGTWYQKAMKVDWDCNQPIFDSDTPMILTTLEGGNLEAKVTIFIDGKYQEVKAVLEKTDEPGKYTADGGKHVTYIIRSHVKDHYIFYSEGQCPGIPVPGMWLVGRDPKNNLEALEDFEKAAGARGLSTESILIPRQSETSSPG

SEQ ID NO: 176 - protein, 152 aa, Artificial Sequence
  lipocalin mutein
ASDEEIQDVSGTWYMKAMKVDWDCNQPIFDSVTPMTLTTLEGGNLEAKVTIFIDGKYQEVKAVLEKTDEPGKYTADGGKHVAYIIRSHVKDHYIFYSEGQCPGIPVPGMWLVGRDPKNNLEALEDFEKAAGARGLSTESILIPRQSETSSPG

SEQ ID NO: 177 - protein, 152 aa, Artificial Sequence
  lipocalin mutein
TSDEEIQDVSGTWYLKAMKVDWDCNQPIFDSVTPMTLTTLEGGNLEAEVTIFIDGKYQEVKAVLEKTDEPGKYTADGGKHVAYIIRSHVKDHYIFYSEGQCPGIPVPGMWLVGRDPKNNLEALEDFEKAAGAHGLSTESILIPRQSETSSPG

SEQ ID NO: 178 - protein, 152 aa, Artificial Sequence
  lipocalin mutein
ATDEEIQDVSGTWYLKAMKVDWDCNQPIFDSVAPMTLTTLEGGNLEAKVTIFIDGKYQEVIAVLEKTDEPGKYTTDGGKHVAYIIRSHVKDHYIFYSEGQCPGIPVPGVWLAGRDPKNNLEALEDFEKAAGARGLSTESILIPRQSETSSPG

SEQ ID NO: 179 - protein, 152 aa, Artificial Sequence
  lipocalin mutein
TSDEEIQDVSGTWYLKAMKVDWDCNQPIFDSDTPMTLTTLEGGNLEAKVTIFIDGKYQEVKAVLEKTDEPGKYTADGGKHVAYIIRSHVKDHYIFYSEGQCPGIPVPGVWLVGRDPKNNLEALEDFEKAAGARGLSTESILIPSQSETSSPG

SEQ ID NO: 180 - protein, 152 aa, Artificial Sequence
  lipocalin mutein
ASDKEIQDVSGTWYLKAMKVDWDCNQPIFDSDTPMTLTTLEGGNLEAKVTIFIDGKYQEVKAVLEKTDEPGKYTADGGKHVAYIIRSHVKDHYIFYSEGQCPGIPVPGLWLVGRDPMNNLEALEDFEKAAGARGLSTESILIPRQSETSSPG

SEQ ID NO: 181 - protein, 152 aa, Artificial Sequence
  lipocalin mutein
ASDEEIRDVSGTWYLKAMKVDWDCNQPIFDSDTPMTLTTLEGGNLEAKVTIFIDGKYQEVKAVLEKTDEPGKYTADGGKHVAYIIRSHVKDHYIFYSEGQCPGIPVPGVWLVGRDPKNNLEALEDFEKAAGARGLRTESILIPWQSETSSPG

SEQ ID NO: 182 - protein, 152 aa, Artificial Sequence
  lipocalin mutein
ASDEEIQDVSGTWYLKAMKVDWDCNQPIFDSDTPMTLTTLEGGNLEAKVTIFIDGKYQEVKAVLEKTDEPGKYTADGGKHVAYIIRSHVKDHYIFYSEGQCPGIPVPGVWLVGRDPKNNLEALEDFEKAAGARGLSTESILIPRQSETSSPG

SEQ ID NO: 183 - protein, 152 aa, Artificial Sequence
  lipocalin mutein
ASDEEIQDVSGTWYLKAMKVDWDCNQPIFDSDTPMTLTTLEGGNLEAKVTIFISGKYQEVKAVLEKTDEPGKYTADGGKHVAYIIRSHVKDHYIFYSEGQCHGIPVPGVWLVGRDPKNNKEALEDFEKAAGARGLSTESILIPRQSETSSPG

SEQ ID NO: 184 - protein, 152 aa, Artificial Sequence
  lipocalin mutein
ASDEEIQDVSGTWYLKAMKVDWDCNQPIFDSDTPMTLTTLEGGNLEAKVTIFIDGKCQEVKAVLEKTDEPGKYTADGGKHVAYIIRSHVKDHYIFYSEGQCPGIPVPGVWLVGRDPKNNLEALEDFEKAAGARGLSTESILIPRQSETCSPG

SEQ ID NO: 185 - protein, 178 aa, Artificial Sequence
  lipocalin mutein
QDSTSDLIPAPPLSKVPLQQNFQDNQFHGKWYVVGFAGNYWLREDKDPGKMQATIYELKEDKSYNVTGVLFPHKKCHYDIVTFVPGSQPGEFTLGWIKSDPGITSDLVRVVSTNYNQHAMVFFKWVFQNREWFYITLYGRTKELTSELKENFIRFSKSLGLPENHIVFPVPIDQCIDG

SEQ ID NO: 186 - protein, 178 aa, Artificial Sequence
  lipocalin mutein
QDSTSDLIPAPPLSKVPLQQNFQDNQFQGKWYVVGFAGNYWLREDKDPGKMQATIYELKKDKSYNVTGVLFPHKECHYDIVTLVPGIQPGEFILGWIKSDPGITSDLVRVVSTDYNQHAMVFFKWVFQNREWFYITLYGRTKELTSELKENFIRFSKSLGLPENHIVFPVPIDQCIDG

SEQ ID NO: 187 - protein, 178 aa, Artificial Sequence
  lipocalin mutein
QDSTSDLIPAPPLSKVPLQQNFQDNQFQGKWYVVGFAGNYWLREDKDPGKMQATIYELKEDKTYNVTGVPFPHKKCHYDIVTFVPGSQPGEFILGWIKSDPGITSDLVRVVSTNYNQHAMVFFKWVFQNREWFYITLYGRTKELTSELKENFIRFSKSLGLPENHIVFPVPIDQCIDG

SEQ ID NO: 188 - protein, 178 aa, Artificial Sequence
  lipocalin mutein
QDSTSDLIPAPPLSKVPLQQNFQDNQFQGKWYVVGFAGNYWLREDKDPGEMQATIYELKEDKSYNVTGVLFPHKKCHYDTVTFVPGRQPGEFILGWIRSDPGITSDLVRVVSTDYNQHAMVFFKWVFQNREWFYITLYGRTKELTSELKENFIRFSKSLGLPENHIVFPVPIDQCIDG

SEQ ID NO: 189 - protein, 178 aa, Artificial Sequence
  lipocalin mutein
QDFTSDLIPAPPLSKVPLQQNFQDNQFQGKWYVVGFAGNYWLREDKDPGKMQATVYELKEDKSYNVTGVLFPHKKCHYDIVTLVPGSQPGEFILGWIRSDPGITSDLVRVVSTNYNQYAMVFFKWVFQNREWFYITLYGRTKELTSELKGNFIRFSKSLGLPENHIVFPVPIDQCIDG

SEQ ID NO: 190 - protein, 178 aa, Artificial Sequence
  lipocalin mutein
QDSTSDLIPAPPLSKVPLQQNFQDNQFHGKWYVVGFAGNYWLRGDKDPGTMQATIYELKEDRSYQVTGVLFPHKECHYDTVILVPGSQPGEFTLGWIKSDPGITSDLVRVVSTDYNQHAMVFFKWVFQNREWFYITLYGRTKELTSELKENFIRFSKSLGLPENHIVFPVPIDQCIDG

SEQ ID NO: 191 - protein, 178 aa, Artificial Sequence
  lipocalin mutein
QDSTSDLIPAPPLSKVPLQQNFQDNQFHGKWYVVGFAGNYWLREDKDPGEMQATIYELKEDKSYQVTGVRFPHKECHYDTVIFVPGSQPGEFTLGWIKSDPGITSDLVRVVSTDYNQYAMVFFKWVFQDREWFYITLYGRTKELTSELKENFIRFSKSLGLPENHIVFPVPIDQCIDG

SEQ ID NO: 192 - protein, 178 aa, Artificial Sequence
  lipocalin mutein
QDSTSDLIPAPPLSKVPLQQNFQDSQFQGKWYVVGFAGNYRLREDKDPGKMQATIYELREDKAYNVTGVPFPHKKCHYDIVTFVPGSQPGEFILGWIKSDPGITSDLVRVVSTNYNQHAMVFFKWVFQNREWFYITLYGRTKELTSELKENFIRFSKSLGLPENHIVFPVPIDQCIDG

SEQ ID NO: 193 - protein, 178 aa, Artificial Sequence
  lipocalin mutein
QDSTSDLIPAPPLSKVPLQQNFQDSQFQGKWYVVGFAGNYRLREDKDPGKMQATIYELREDKAYQVTGVPFPHKKCHDDIVIFVPGSQPGEFILGWIKSDPGITSDLVRVVSTDYNQHAMVFFKWVFQNREWFYITLYGRTKALTSELKENFIRFSKSLGLPEDHIVFPVPIDQCIDG

SEQ ID NO: 194 - protein, 178 aa, Artificial Sequence
  lipocalin mutein
QDSTSDLIPAPPLSKVPLQQNFQDSQFQGKWYVVGFAGNYRLREDKDPGKMQATIYELREDKAYQVTGVLFPHKKCHDDIVIFVPGSQPGEFILGWIKSDPGITSDLVRVVSTDYNQHAMVFFKWVFQNREWFYITLYGRTKALTSELKENFIRFSKSLGLPEDHIVFPVPIDQCIDG

SEQ ID NO: 195 - protein, 178 aa, Artificial Sequence
  lipocalin mutein
QDPTSDLIPAPPLSKVPLQQNFQDSQFQGKWYVVGFAGNYRLREDKDPGEMQATIYELREDKAYQVTGVPFPHKKCHYDTVIFVPGSQPGEFILGWIKSDPGITSDLVRVVSTDYNQHAMVFFKWVFQNREWFYITLYGRTKELTSELKENFIRFSKSLGLPENHIVFPVPIDQCIDG

SEQ ID NO: 196 - protein, 178 aa, Artificial Sequence
  lipocalin mutein
QDSTSDLIPAPPLSKVPLQQNFQDSRFQGKWYVVGFAGNYRLREDKDPGKMQATIYELREDKAYQVTGVPFPHKKCHHDTVIFVPGSQPGEFILGWIKSDPGITSDLVRVVSTNYNQHAMVFFKWVFQNREWFYITLYGRTKELTSELKENFIRFSKSLGLPEDHIVFPVPIDQCIDG

SEQ ID NO: 197 - protein, 178 aa, Artificial Sequence
  lipocalin mutein
QDSTSDLIPAPPLSKVPLQQDFQDSQFQGKWYVVGFAGNYRLREDKDPGKMQATIYELREDKAYQVTGVPFPHKKCHDDTVIFVPGSQPGEFILGWIKSDPGITSDLVRVVSTNYNQHAMVFFKWVFQNREWFYITLYGRTKELTSELKENFIRFSKSLGLPEDHIVFPVPIDQCIDG

SEQ ID NO: 198 - protein, 178 aa, Artificial Sequence
  lipocalin mutein
QDSTSDLIPAPPLSKVPLQQNFQDSQFQGKWYVVGFAGNYRLREDKDPGKMQATIYELREDKAYQVTGVPFPHKECHYDTVIFVPGSQPGEFILGWIKSDPGITSDLVRVVSTNYNQHAMVFFKWVFQDREWFYITLYGRTKELTSELKENFIRFSKSLGLPENHIVFPVPIDQCIDG

SEQ ID NO: 199 - protein, 178 aa, Artificial Sequence
  lipocalin mutein
QDSTSDLIPAPPLSKVPLQQNFQDSQFQGKWYVVGFAGNYRLREDKDPGEMQATIYELREDKAYRVTGVPFPHKECHYDIVVFVPGSQPGEFILGWIKSDPGITSDLARVVSTNYNQHAMVFFKWVFQDREWFYITLYGRTKELTSELKENFIRFSKSLGLPEDHIVFPAPIDQCIDG

SEQ ID NO: 200 - protein, 178 aa, Artificial Sequence
  lipocalin mutein
QDSTSDLIPAPPLSKVPLQQNFQDSQFQGKWYVVGFAGNYRLREDKDPGKMQATIYELREDKAYQVTGVLFPHKKCHDDIVIFVPGSQPGEFILGWIKSDPGITSDLVRVVSTDYNQHAMVFFKWVFQNREWFYITLYGRTKELTSELKENFIRFSKSLGLPEDHIVFPVPIDQCIDG

SEQ ID NO: 201 - protein, 178 aa, Artificial Sequence
  lipocalin mutein
QDSTSDLIPAPPLSKVPLQQNFQDSQFQGKWYVVGFAGNYRLREDKDPGKMQATIYELREDKAYQVTGVLFPHKKCHDDTVIFVPGSQPGEFILGWIKSDPGITSDLVRVVSTDYNQHAMVFFKWVFQNREWFYITLYGRTKELTSELKENFIRFSKSLGLPEDHIVFPVPIDQCIDG

SEQ ID NO: 202 - protein, 178 aa, Artificial Sequence
  lipocalin mutein
QDSTSDLIPAPPLSKVPLQQNFQDSQFQGKWYVVGFAGNYRLREDKDPGKMQATIYELREDKAYQVTGVPFPHKKCHDDTVIFVPGSQPGEFILGWIKSDPGITSDLVRVVSTDYNQHAMVFFKWVFQNREWFYITLYGRTKELTSELKENFIRFSKSLGLPEDHIVFPVPIDQCIDG

SEQ ID NO: 203 - protein, 244 aa, Artificial Sequence
  fusion protein
QDSTSDLIPAPPLSKVPLQQNFQDSQFQGKWYVVGFAGNYRLREDKDPGKMQATIYELREDKAYQVTGVLFPHKKCHDDIVIFVPGSQPGEFILGWIKSDPGITSDLVRVVSTDYNQHAMVFFKWVFQNREWFYITLYGRTKALTSELKENFIRFSKSLGLPEDHIVFPVPIDQCIDGGGGGSGGGGSGGGGSGVRLWATRQAMLGQVHEVPEGWLIFVAEQEELYVRVQNGFRKVQLEARTPL

SEQ ID NO: 204 - protein, 133 aa, human
QVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

(19) United States
(12) Patent Application Publication
(10) Pub. No.: US 2023/0227568 A1
(43) Pub. Date: Jul. 20, 2023

(54) MULTIMERIC IMMUNOMODULATOR TARGETING 4-1BB

(71) Applicant: Pieris Pharmaceuticals GmbH, Hallbergmoos (DE)

(72) Inventors: Janet PEPER-GABRIEL, Hallbergmoos (DE); Josef PRASSLER, Hallbergmoos (DE); Timo EICHNER, Hallbergmoos (DE); Stefan GRUENER, Hallbergmoos (DE); Ahmed MOUSA, Hallbergmoos (DE); Shane OLWILL, Hallbergmoos (DE)

(21) Appl. No.: 18/000,651

(22) PCT Filed: Jun. 4, 2021
(86) PCT No.: PCT/EP2021/065020

§ 371 Date: Dec. 2, 2022

(30) Foreign Application Priority Data
Jun. 5, 2020 (EP) 20178414.7
Jun. 8, 2020 (EP) 20178721.5

(51) Int. Cl. C07K 16/28; C07K 16/18; C07K 14/78; A61P 35/00; C07K 16/30; A61K 35/06

(52) U.S. Cl. CPC C07K 16/2878; C07K 16/18; C07K 16/2827; C07K 14/78; A61P 35/00; C07K 16/303; A61K 35/06; C07K 2317/622; C07K 2317/31; C07K 2319/30; C07K 2317/92; A61K 38/00

(57) ABSTRACT

The disclosure provides multimeric proteins comprising three, four, or more monomer polypeptides, each comprising a first 4-1BB-targeting moiety, an oligomerization moiety, and optionally a linker. The monomer polypeptide may further comprise one or more additional targeting moieties. The oligomerization moiety promotes the trimerization, tetramerization, or higher state of oligomerization of the monomer polypeptides. Such multimeric proteins can be used in many pharmaceutical applications, for example, as anti-cancer agents and/or immune modulators. The present disclosure also concerns methods of making the multimeric proteins described herein as well as compositions comprising such multimeric proteins. The present disclosure further relates to nucleic acid molecules encoding such multimeric proteins and methods for the generation of such multimeric proteins and nucleic acid molecules. In addition, the application discloses therapeutic and/or diagnostic uses of such multimeric proteins as well as compositions comprising one or more of such multimeric proteins.

### I. BACKGROUND

Cluster of differentiation 137 or CD137 (also known as 4-11BB or TNFRS9) is a co-stimulatory immune receptor and a member of the tumor necrosis factor receptor (TNFR) super-family. It is primarily expressed on activated CD4+ and CD8+ T cells, activated B cells, and natural killer (NK) cells but can also be found on resting monocytes and dendritic cells (Li and Liu, 2013), or endothelial cells (Snell et al., 2011). 4-1BB plays an important role in regulation of the immune response and thus is a target for cancer immunotherapy. 4-1BB ligand (4-1BBL) is the only known natural ligand of 4-11BB, and is constitutively expressed on several types of antigen presenting cells, such as activated B cells, monocytes, and splenic dendritic cells, and can be induced on T lymphocytes.

The benefit of 4-11BB co-stimulation for the elimination of cancer cells has been demonstrated in a number of in vivo models. The forced expression of 4-1BBL on a tumor, for example, leads to tumor rejection (Melero et al., 1998). Likewise, the forced expression of an anti-4-1BB scFv on a tumor leads to a CD4^{+ T cell and NK-cell dependent elimination of the tumor (Yang et al.,} 2007, Zhang et al., 2006, Ye et al., 2002). A systemically administered anti-4-11BB antibody has also been demonstrated to lead to retardation of tumor growth (Martinet et al., 2002). It has also been shown that 4-11BB is an excellent marker for naturally occurring tumor-reactive T cells in human tumors (Ye et al., 2014), and that anti-4-1BB antibodies can be employed to improve the expansion and activity of CD8^{+ melanoma tumor-infiltrating lymphocytes for the application in adoptive T cell therapy (Chacon et al.,} 2013). The preclinical demonstration of the potential therapeutic benefit of 4-11BB co-stimulation has spurred the development of therapeutic antibodies targeting 4-11BB, including BMS-663513 (described in U.S. Pat. No. 7,288,638) and PF-05082566 (Fisher et al., 2012).

4-1BBL is a trimeric protein that exists as a membrane-bound form which can be proteolytically cleaved into a soluble trimeric ligand. The ability of soluble 4-1BBL to activate 4-11BB, e.g., on 4-1BB-expressing lymphocytes is limited, however, and large concentrations are required to elicit an effect (Wyzgol et al., 2009), providing evidence that larger scale clustering of 4-11BB on the cell surface is required for inducing intracellular 4-11BB (Wyzgol et al., 2009, Rabu et al., 2005). The natural way of activation of 4-11BB is via the engagement of a 4-1BB-positive cell with a 4-1BBL-positive cell. 4-11BB activation is then thought to be induced by clustering through 4-1BBL on the opposing cell, leading to signaling via TRAF1, 2 and 3 (Snell et al., 2011, Yao et al., 2013) and further concomitant downstream effects in the 4-1BB-positive T cell. In the case of T cells activated by recognition of their respective cognate targets, the effects elicited by co-stimulation of 4-1BB are a further enhanced activation, enhanced survival and proliferation, the production of pro-inflammatory cytokines and an improved capacity to kill.

The present disclosure provides, among other things, novel approaches for stimulating 4-1BB via one or more 4-1BB-targeting multimeric proteins that enable the high level of 4-1BB clustering in an FcγR-independent manner.

### II. Definitions

The following list defines terms, phrases, and abbreviations used throughout the instant specification. All terms listed and defined herein are intended to encompass all grammatical forms.

As used herein, unless otherwise specified, “4-1BB” means human 4-1BB (hu4-1BB). Human 4-1BB means a full-length protein defined by UniProt 007011, a fragment thereof, or a variant thereof. Human 4-1BB is encoded by the gene TNFRSF9. 4-1BB is also known as cluster of differentiation 137 (CD137) or tumor necrosis factor receptor superfamily member 9 (TNFRSF9), which are used interchangeably. Cynomolgus 4-1BB (cy4-1BB) refers to the 4-1BB of cynomolgus monkeys. In some particular embodiments, 4-1BB of non-human species, e.g., cynomolgus 4-1BB and mouse 4-1BB, is used.

As used herein, unless otherwise specified, “Glypican-3” or “GPC3” means human GPC3 (huGPC3). Human GPC3 means a full-length protein defined by UniProt P51654, a fragment thereof, or a variant thereof. Human OPC3 is encoded by the gene GPC3. In some particular embodiments, GPC3 of non-human species, e.g., cynomolgus GPC3 and mouse GPC3, is used.

As used herein, unless otherwise specified, “OX40” means human OX40 (huOX40). Human OX40 means a full-length protein defined by UniProt P43489, a fragment thereof, or a variant thereof. Human OX40 is encoded by the gene TNFRSF4. OX40 is also known as cluster of differentiation 134 (CD134) or tumor necrosis factor receptor superfamily member 4 (TNFRSF4), which are used interchangeably. Cynomolgus OX40 (cyOX40) refers to the OX40 of cynomolgus monkeys. In some particular embodiments, OX40 of non-human species, e.g., cynomolgus OX40 and mouse OX40, is used.

As used herein, unless otherwise specified, “programmed cell death 1 ligand 1” or “PD-L1” means human PD-L1 (huPD-L1). Human PD-L1 means a full-length protein defined by UniProt Q9NZQ7, a fragment thereof, or a variant thereof. Human PD-L1 is encoded by the gene CD274. PD-L1 is also known as cluster of differentiation 274 (CD274) or B7 homolog 1 (B7-H1). In some particular embodiments, PD-L1 of non-human species, e.g., cynomolgus PD-L1 and mouse PD-L1, is used.

As used herein, “binding affinity” describes the ability of a biomolecule (e.g., a polypeptide or a protein) of the disclosure (e.g., a lipocalin mutein, an antibody, a fusion protein, a multimeric protein, or any other peptide or protein) to bind a selected target and form a complex. Binding affinity is measured by a number of methods known to those skilled in the art including, but not limited to, fluorescence titration, enzyme-linked immunosorbent assay (ELISA)-based assays, including direct and competitive ELISA, calorimetric methods, such as isothermal titration calorimetry (ITC), and surface plasmon resonance (SPR). These methods are well-established in the art and some examples of such methods are further described herein. Binding affinity is thereby reported as a value of dissociation constant (K_{D}), half maximal effective concentration (EC₅₀), or half maximal inhibitory concentration (IC₅₀) measured using such methods. A lower K_{D}, EC₅₀, or IC₅₀ value reflects better (higher) binding ability (affinity). Accordingly, the binding affinities of two biomolecules toward a selected target can be measured and compared. When comparing the binding affinities of two biomolecules toward the selected target, the term “about the same,” “substantially the same” or “substantially similar” means one biomolecule has a binding affinity reported as a K_{D}, an EC₅₀, or an IC₅₀ value that is identical or similar to that of another molecule within the experimental variability of the binding affinity measurement. The experimental variability of the binding affinity measurement is dependent upon the specific method used and is known to those skilled in the art.

As used herein, the term “substantially” may also refer to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

As used herein, the term “detect,” “detection,” “detectable,” or “detecting” is understood both on a quantitative and a qualitative level, as well as a combination thereof. It thus includes quantitative, semi-quantitative, and qualitative measurements performed on a biomolecule of the disclosure.

As used herein, “detectable affinity” generally means the binding ability between a biomolecule and its target, reported by a K_{D}, EC₅₀, or IC₅₀ value, is at most about 10→M or lower. A binding affinity, reported by a K_{D}, EC₅₀, or IC₅₀ value, higher than 10→M is generally no longer measurable with common methods such as ELISA and SPR and is therefore of secondary importance.

It is noted that the complex formation between the biomolecule of the disclosure and its target is influenced by many different factors such as the concentrations of the respective target, the presence of competitors, pH and the ionic strength of the buffer system used, the experimental method used for determination of the binding affinity (e.g., fluorescence titration, competitive ELISA (also called competition ELISA), and surface plasmon resonance), and even the mathematical algorithm used for evaluation of the experimental data. Therefore, it is clear to the skilled person that binding affinity reported by a K_{D}, EC₅₀, or IC₅₀ value may vary within a certain experimental range, depending on the method and experimental setup. This means that there may be a slight deviation in the measured K_{D}, EC₅₀, or IC₅₀ values or a tolerance range depending, for example, on whether such values were determined by ELISA (including direct or competition ELISA), by SPR, or by another method.

As used herein, “specific for,” “specific binding,” “specifically bind,” or “binding specificity” relates to the ability of a biomolecule to discriminate between the desired target (for example, 4-1BB, OX40, PD-L1 and GPC3) and one or more reference targets (for example, cellular receptor for neutrophil gelatinase-associated lipocalin). It is understood that such specificity is not an absolute but a relative property and can be determined, for example, in accordance with SPR, western blots, ELISA, fluorescence activated cell sorting (FACS), radioimmunoassay (RIA), electrochemiluminescence (ECL), immunoradiometric assay (IRMA), ImmunoHistoChemistry (IHC), and peptide scans.

When used herein in the context of the multimeric protein of the present disclosure that bind to 4-1BB, OX40, PD-L1 and/or GPC3, the term “specific for,” “specific binding,” “specifically bind,” or “binding specificity” means that the multimeric protein binds to, reacts with, or is directed against 4-1BB, OX40, PD-L1 and/or GPC3, as described herein, but does not essentially bind another protein. The term “another protein” includes any proteins that are not 4-1BB, OX40, PD-L1 or GPC3 or proteins closely related to or being homologous to 4-1BB, OX40, PD-L1 or GPC3. However, 4-1BB, OX40, PD-L1 or GPC3 from species other than human and fragments and/or variants of 4-1BB, OX40, PD-L1 or GPC3 are not excluded by the term “another protein.” The term “does not essentially bind” means that the multimeric proteins of the present disclosure bind another protein with lower binding affinity than 4-1BB, OX40, PD-L1 and/or GPC3, i.e., show a cross-reactivity of less than 30%, preferably less than 20%, more preferably less than 10%, particularly preferably less than 9, 8, 7, 6, or 5%. Whether the multimeric protein specifically reacts as defined herein above can easily be tested, inter alia, by comparing the reaction of a multimeric protein of the present disclosure with 4-1BB, OX40, PD-L1 and/or GPC3 and the reaction of said multimeric protein with (an)other protein(s).

As used herein, the term “lipocalin” refers to a monomeric protein of approximately 18-20 kDa in weight, having a cylindrical p-pleated sheet supersecondary structural region comprising a plurality of p-strands (preferably eight p-strands designated A to H) connected pair-wise by a plurality of (preferably four) loops at one end to thereby comprise a ligand-binding pocket and define the entrance to the ligand-binding pocket. Preferably, the loops comprising the ligand-binding pocket used in the present invention are loops connecting the open ends of β-strands A and B, C and D, E and F, and G and H, and are designated loops AB, CD, EF, and GH. It is well-established that the diversity of said loops in the otherwise rigid lipocalin scaffold gives rise to a variety of different binding modes among the lipocalin family members, each capable of accommodating targets of different sizes, shape, and chemical character (reviewed, e.g. in Skerra, 2000, Flower et al., 2000, Flower, 1996). It is understood that the lipocalin family of proteins has naturally evolved to bind a wide spectrum of ligands, sharing unusually low levels of overall sequence conservation (often with sequence identities of less than 20%) yet retaining a highly conserved overall folding pattern. The correspondence between positions in various lipocalins is also well-known to one of skill in the art (see, e.g., U.S. Pat. No. 7,250,297). Proteins falling in the definition of “lipocalin” as used herein include, but are not limited to, human lipocalins including tear lipocalin (Tlc, Lcn1), Lipocalin-2 (Lcn2) or neutrophil gelatinase-associated lipocalin (NGAL), apolipoprotein D (ApoD), apolipoprotein M, α₁-acid glycoprotein 1, α₁-acid glycoprotein 2, α₁-microglobulin, complement component 8γ, retinol-binding protein (RBP), the epididymal retinoic acid-binding protein, glycodelin, odorant-binding protein IIa, odorant-binding protein IIb, lipocalin-15 (Lcn15), and prostaglandin D synthase.

As used herein, unless otherwise specified, “tear lipocalin” refers to human tear lipocalin (hTlc) and further refers to mature human tear lipocalin. The term “mature” when used to characterize a protein means a protein essentially free from the signal peptide. A “mature hTlc” of the instant disclosure refers to the mature form of human tear lipocalin, which is free from the signal peptide. Mature hTlc is described by residues 19-176 of the sequence deposited with the SWISS-PROT Data Bank under Accession Number P31025, and the amino acid of which is indicated in SEQ ID NO: 1.

As used herein, “Lipocalin-2” or “neutrophil gelatinase-associated lipocalin” refers to human Lipocalin-2 (hLcn2) or human neutrophil gelatinase-associated lipocalin (hNGAL) and further refers to the mature human Lipocalin-2 or mature human neutrophil gelatinase-associated lipocalin. The term “mature” when used to characterize a protein means a protein essentially free from the signal peptide. A “mature hNGAL” of the instant disclosure refers to the mature form of human neutrophil gelatinase-associated lipocalin, which is free from the signal peptide. Mature hNGAL is described by residues 21-198 of the sequence deposited with the SWISS-PROT Data Bank under Accession Number P80188, and the amino acid of which is indicated in SEQ ID NO: 2.

As used herein, a “native sequence” refers to a protein or a polypeptide having a sequence that occurs in nature or having a wild-type sequence, regardless of its mode of preparation. Such native sequence protein or polypeptide can be isolated from nature or can be produced by other means, such as by recombinant or synthetic methods.

The “native sequence lipocalin” refers to a lipocalin having the same amino acid sequence as the corresponding polypeptide derived from nature. Thus, a native sequence lipocalin can have the amino acid sequence of the respective naturally-occurring (wild-type) lipocalin from any organism, in particular, a mammal. The term “native sequence”, when used in the context of a lipocalin specifically encompasses naturally-occurring truncated or secreted forms of the lipocalin, naturally-occurring variant forms such as alternatively spliced forms and naturally-occurring allelic variants of the lipocalin. The terms “native sequence lipocalin” and “wild-type lipocalin” are used interchangeably herein.

As used herein, a “mutein,” a “mutated” entity (whether protein or nucleic acid), or “mutant” refers to the exchange, deletion, or insertion of one or more amino acids or nucleotides, compared to the naturally-occurring (wild-type) protein or nucleic acid. Said term also includes fragments of a mutein as described herein. The present disclosure explicitly encompasses lipocalin muteins, as described herein, having a cylindrical p-pleated sheet supersecondary structural region comprising eight p-strands connected pair-wise by four loops at one end to thereby comprise a ligand-binding pocket and define the entrance of the ligand-binding pocket, wherein at least one amino acid of each of at least three of said four loops has been mutated as compared to the native sequence lipocalin. Lipocalin muteins of the present disclosure preferably have the function of binding 4-1BB, OX40 or GPC3 as described herein.

As used herein, the term “fragment,” in connection with the lipocalin muteins of the disclosure, refers to proteins or polypeptides derived from full-length mature hTlc or hNGAL or lipocalin muteins that are N-terminally and/or C-terminally truncated, i.e., lacking at least one of the N-terminal and/or C-terminal amino acids. Such fragments may include at least 10 or more, such as 20 or 30 or more consecutive amino acids of the primary sequence of mature hTlc or hNGAL or the lipocalin mutein it is derived from and are usually detectable in an immunoassay of mature hTlc or hNGAL. Such a fragment may lack up to 2, up to 3, up to 4, up to 5, up to 10, up to 15, up to 20, up to 25, or up to 30 (including all numbers in between) of the N-terminal and/or C-terminal amino acids. As an illustrative example, such a fragment may lack the one, two, three, or four N-terminal (His-His-Leu-Leu) and/or one or two C-terminal amino acids (Ser-Asp) of mature hTlc. It is understood that the fragment is preferably a functional fragment of mature hTlc or hNGAL or the lipocalin mutein from which it is derived, which means that it preferably retains the binding specificity, preferably to 4-1EE, OX40 or GPC3, of mature hTlc/hNGAL or lipocalin mutein it is derived from. As an illustrative example, such a functional fragment may comprise at least amino acids at positions 5-153, 5-150, 9-148, 12-140, 20-135, or 26-133 corresponding to the linear polypeptide sequence of mature hTlc. As another illustrative example, such a functional fragment may comprise at least amino acids at positions 13-157, 15-150, 18-141, 20-134, 25-134, or 28-134 corresponding to the linear polypeptide sequence of mature hNGAL.

A “fragment” with respect to the corresponding target, such as 4-1BB, OX40, PD-L1 or GPC3, of a multimeric protein of the disclosure, refers to N-terminally and/or C-terminally truncated target protein such as 4-1BB, OX40, PD-L1 or GPC3, or protein domains of a target protein such as 4-1BB, OX40, PD-L1 or GPC3. Fragments of 4-1BB, OX40, PD-L1 or GPC3 as described herein retain the capability of the full-length 4-1BB, OX40, PD-L1 or GPC3 to be recognized and/or bound by a multimeric protein of the disclosure. As an illustrative example, the fragment may be an extracellular domain of 4-1BB, OX40, PD-L1 or GPC3. As an illustrative example, such an extracellular domain of human 4-1BB may comprise residues 24-186 of UniProt 007011 or residues 1-163 of SEQ ID NO: 4. Such an extracellular domain may comprise amino acids of the extracellular subdomains of 4-1BB, such as the individual or combined amino acid sequences of domain 1 (residues 24-45 of UniProt 007011), domain 2 (residues 46-86 of UniProt 007011), domain 3 (87-118 of UniProt 007011) and domain 4 (residues 119-159 of UniProt 007011). An extracellular domain of cynomolgus 4-1BB may comprise residues 1-163 of SEQ ID NO: 6.

As used herein, the term “variant” relates to derivatives of a protein or polypeptide that include mutations, for example by substitutions, deletions, insertions, and/or chemical modifications of an amino acid sequence or nucleotide sequence. In some embodiments, such mutations and/or chemical modifications do not reduce the functionality of the protein or peptide. Such substitutions may be conservative, i.e., an amino acid residue is replaced with a chemically similar amino acid residue. Examples of conservative substitutions are the replacements among the members of the following groups: 1) alanine, serine, threonine, and valine; 2) aspartic acid, glutamic acid, glutamine, and asparagine, and histidine; 3) arginine, lysine, glutamine, asparagine, and histidine; 4) isoleucine, leucine, methionine, valine, alanine, phenylalanine, threonine, and proline; and 5) isoleucine, leucine, methionine, phenylalanine, tyrosine, and tryptophan. Such variants include proteins or polypeptides, wherein one or more amino acids have been substituted by their respective D-stereoisomers or by amino acids other than the naturally occurring 20 amino acids, such as, for example, ornithine, hydroxyproline, citrulline, homoserine, hydroxylysine, norvaline. Such variants also include, for instance, proteins or polypeptides in which one or more amino acid residues are added or deleted at the N- and/or C-terminus. Generally, a variant has at least about 50%, 60%, 70%, 75%, 80%, 85%, 90%, 92%, 95%, 98%, or at least about 99% amino acid sequence identity with the native sequence protein or polypeptide. A variant preferably retains the biological activity, e.g. binding the same target, of the protein or polypeptide it is derived from.

The term “variant”, as used herein with respect to the corresponding protein target, such as 4-1BB, OX40, PD-L1 or GPC3, of a multimeric protein of the disclosure, relates to a protein target, such as 4-1BB, OX40, PD-L1 or GPC3, or fragment thereof, respectively, that has one or more, such as 1, 2, 3, 4, 5, 6, 7, 8, 9, 10, 12, 14, 16, 18, 20, 22, 24, 26, 28, 30, 40, 50, 60, 70, 80 or more, amino acid substitutions, deletions and/or insertions in comparison to the native sequence of the protein target, such as 4-1BB as deposited with UniProt 007011, OX40 as deposited with UniProt P43489, PD-L1 as deposited with UniProt Q9NZQ7 or GPC3 as deposited with UniProt P51654, as described herein. A 4-1BB, OX40, PD-L1 or GPC3 variant, respectively, has preferably an amino acid sequence identity of at least 50%, 60%, 70%, 80%, 85%, 90% or 95% with a wild-type 4-1BB, OX40, PD-L1 or GPC3, respectively. A 4-1BB, OX40, PD-L1 or GPC3 variant as described herein retains the ability to bind multimeric proteins specific to 4-1BB, OX40, PD-L1 and/or GPC3 disclosed herein.

The term “variant”, as used herein with respect to a lipocalin mutein, relates to a lipocalin mutein or fragment thereof of the disclosure, wherein the sequence has mutations, including substitutions, deletions, and insertions, and/or chemical modifications. A variant of lipocalin mutein as described herein retains the biological activity, e.g., binding to 4-1BB, OX40 or GPC3, of the lipocalin mutein from which it is derived. Generally, a lipocalin mutein variant has at least about 50%, 60%, 70%, 75%, 80%, 85%, 90%, 92%, 95%, 98%, or 99% amino acid sequence identity with the lipocalin mutein from which it is derived.

As used herein, the term “mutagenesis” refers to the introduction of mutations into a polynucleotide or amino acid sequence. Mutations are preferably introduced under experimental conditions such that the amino acid naturally occurring at a given position of the protein or polypeptide sequence can be altered, for example substituted by at least one amino acid. The term “mutagenesis” also includes the (additional) modification of the length of sequence segments by deletion or insertion of one or more amino acids. Thus, it is within the scope of the disclosure that, for example, one amino acid at a chosen sequence position is replaced by a stretch of three amino acids, leading to an addition of two amino acid residues compared to the length of the respective segment of the native protein or polypeptide amino acid sequence. Such an insertion or deletion may be introduced independently from each other in any of the sequence segments that can be subjected to mutagenesis in the disclosure. In one exemplary embodiment of the disclosure, an insertion may be introduced into an amino acid sequence segment corresponding to the loop AB of the native sequence lipocalin (cf. International Patent Publication No. WO 2005/019256, which is incorporated by reference in its entirety herein).

As used herein, the term “random mutagenesis” means that no predetermined mutation (alteration of an amino acid) is present at a certain sequence position but that at least two amino acids can be incorporated with a certain probability at a predefined sequence position during mutagenesis.

As used herein, the term “sequence identity” or “identity” denotes a property of sequences that measures their similarity or relationship. The term “sequence identity” or “identity” as used in the present disclosure means the percentage of pair-wise identical residues—following (homologous) alignment of a sequence of a protein or polypeptide of the disclosure with a sequence in question—with respect to the number of residues in the longer of these two sequences. Sequence identity is measured by dividing the number of identical amino acid residues by the total number of residues and multiplying the product by 100.

As used herein, the term “sequence homology” or “homology” has its usual meaning and homologous amino acid includes identical amino acids as well as amino acids which are regarded to be conservative substitutions at equivalent positions in the linear amino acid sequence of a protein or polypeptide of the disclosure (e.g., any antibodies, antibody fragments or derivatives, multimeric proteins, or lipocalin muteins of the disclosure).

A skilled artisan will recognize available computer programs, for example BLAST (Altschul et al., 1997), BLAST2 (Altschul et al., 1990), and Smith-Waterman (Smith and Waterman, 1981), for determining sequence homology or sequence identity using standard parameters. The percentage of sequence homology or sequence identity can, for example, be determined herein using the program BLASTP, version 2.2.5 (Nov. 16, 2002; Altschul et al., 1997). In this embodiment, the percentage of homology is based on the alignment of the entire protein or polypeptide sequences (matrix: BLOSUM 62; gap costs: 11.1; cutoff value set to 10⁻³) including the propeptide sequences, preferably using the wild-type protein scaffold as reference in a pairwise comparison. It is calculated as the percentage of numbers of “positives” (homologous amino acids) indicated as result in the BLASTP program output divided by the total number of amino acids selected by the program for the alignment.

Specifically, in order to determine whether the amino acid sequence of a lipocalin mutein is different from that of a reference (wild-type) lipocalin with regard to a certain position in the amino acid sequence of the reference (wild-type) lipocalin, a skilled artisan can use means and methods well-known in the art, e.g., alignments, either manually or by using computer programs such as BLAST 2.0, which stands for Basic Local Alignment Search Tool, or ClustalW, or any other suitable program which is suitable to generate sequence alignments. Accordingly, the amino acid sequence of a reference (wild-type) lipocalin can serve as “subject sequence” or “reference sequence”, while the amino acid sequence of a lipocalin mutein serves as “query sequence.” The terms “wild-type sequence,” “reference sequence,” and “subject sequence” are used interchangeably herein. A preferred wild-type sequence of a lipocalin is the sequence of hTLc as shown in SEQ ID NO: 1 or hNGAL as shown in SEQ ID NO: 2.

“Gaps” are spaces in an alignment that are the result of additions or deletions of amino acids. Thus, two copies of exactly the same sequence have 100% identity, but sequences that are less highly conserved, and have deletions, additions, or replacements, may have a lower degree of sequence identity.

As used herein, the term “position” means the position of either an amino acid within an amino acid sequence disclosed herein or the position of a nucleotide within a nucleic acid sequence disclosed herein. It is to be understood that when the term “correspond” or “corresponding” is used herein in the context of the amino acid sequence positions of one or more lipocalin muteins, a corresponding position is not only determined by the number of the preceding nucleotides or amino acids. Accordingly, the absolute position of a given amino acid in accordance with the disclosure may vary from the corresponding position due to deletion or addition of amino acids elsewhere in a (mutant or wild-type) lipocalin. Similarly, the absolute position of a given nucleotide in accordance with the present disclosure may vary from the corresponding position due to deletions or additional nucleotides elsewhere in a mutein or wild-type lipocalin 5′-untranslated region (UTR) including the promoter and/or any other regulatory sequences or gene regions (including exons and introns).

A “corresponding position” in accordance with the disclosure may be the sequence position that aligns to the sequence position it corresponds to in a pairwise or multiple sequence alignment according to the present disclosure. It is preferably to be understood that for a “corresponding position” in accordance with the disclosure, the absolute positions of nucleotides or amino acids may differ from adjacent nucleotides or amino acids but said adjacent nucleotides or amino acids which may have been exchanged, deleted, or added may be comprised by the same one or more “corresponding positions”.

In addition, for a corresponding position in a lipocalin mutein based on a reference sequence in accordance with the disclosure, it is preferably to be understood that the positions of nucleotides or amino acids of a lipocalin mutein can structurally correspond to the positions elsewhere in a reference lipocalin (wild-type lipocalin) or another lipocalin mutein, even if they may differ in the absolute position numbers, as appreciated by the skilled in light of the highly-conserved overall folding pattern among lipocalins.

As used interchangeably herein, the terms “conjugate,” “conjugation,” “fuse,” “fusion,” or “linked” refer to the joining together of two or more moieties, through any forms of covalent or non-covalent linkage, by means including, but not limited to, genetic fusion, chemical conjugation, coupling through a linker or a cross-linking agent, and non-covalent association.

The term “multimeric protein” or “multimer” as used herein refers to a protein complex of two or more associated “monomer polypeptides”. Monomer polypeptides in a multimeric protein are linked by non-covalent bonding. In some embodiment, a multimeric protein as described herein comprises two, there, four, five, or more monomer polypeptides. In some embodiments, a multimeric protein may be homomultimeric, where the monomer polypeptides of the multimeric protein are identical. In some embodiments, a multimeric protein may be heteromultimeric, where the monomer polypeptides of the multimeric protein are different.

In some embodiments, a multimeric protein as described herein comprises two or more monomer polypeptides, each comprising a 4-1BB-targeting moiety and an oligomerization moiety and optionally one or more additional targeting moieties. In some embodiments, a multimeric protein as described herein comprises three or more monomer polypeptides, each comprising a 4-1BB-targeting moiety and an oligomerization moiety and optionally one or more additional targeting moieties. In some embodiments, a multimeric protein as described herein comprises four or more monomer polypeptides, each comprising a 4-1BB-targeting moiety and an oligomerization moiety and optionally one or more additional targeting moieties. Within the monomer polypeptide, these moieties may be linked by covalent or non-covalent linkage. Preferably, the monomer polypeptide is a translational fusion polypeptide between the two or more moieties. The translational fusion polypeptide may be generated by genetically engineering the coding sequence for one moiety in a reading frame with the coding sequence of a further moiety. Both moieties may be interspersed by a nucleotide sequence encoding a linker. However, the moieties of a monomer polypeptide of the present disclosure may also be linked through chemical conjugation. The moieties forming the monomer polypeptide are typically linked to each other as follows: C-terminus of one moiety to N-terminus of another moiety, or C-terminus of one moiety to C-terminus of another moiety, or N-terminus of one moiety to N-terminus of another moiety, or N-terminus of one moiety to C-terminus of another moiety. The moieties of the monomer polypeptide can be linked in any order and may include more than one of any of the constituent moieties.

An “oligomerization moiety” or “multimerization moiety” as disclosed herein promotes the assembly of monomer polypeptides into multimeric proteins. In some embodiments, an oligomerization moiety promotes trimerization, tetramerization, or higher oligomeric state of monomer polypeptides. In some preferred embodiments, an oligomerization moiety promotes trimerization of monomer polypeptides.

As used herein, the term “moiety” of a monomer polypeptide disclosed herein refers to a single protein, polypeptide, or peptide, which may form a stable structure by itself and define a unique function. In some embodiments, a preferred moiety of the disclosure is a lipocalin mutein. In some embodiments, a preferred moiety of the disclosure is a full-length antibody or an antigen-binding domain or derivative thereof, such as a single-chain variable fragment (scFv). In some embodiments, a preferred moiety of the disclosure is an oligomerization moiety.

A “linker” that may be comprised by a monomer polypeptide of the present disclosure joins together two or more moieties of a monomer polypeptide as described herein. The linkage can be covalent or non-covalent. A preferred covalent linkage is via a peptide bond, such as a peptide bond between amino acids. A preferred linker is a peptide linker. Accordingly, in a preferred embodiment, said linker comprises one or more amino acids, such as 1, 2, 3, 4, 5, 6, 7, 8, 9, 10, 11, 12, 13, 14, 15, 16, 17, 18, 19, 20 or more amino acids. Preferred peptide linkers are described herein, including glycine-serine (GS) linkers, glycosylated GS linkers, proline-alanine-serine polymer (PAS) linkers, helix-forming linkers, and rigid linkers. Exemplary peptide linkers are shown in SEQ ID NOs: 12-28. Other preferred linkers include chemical linkers.

A “sample” is defined as a biological sample taken from any subject. Biological samples include, but are not limited to, blood, serum, urine, feces, semen, or tissue, including tumor tissue.

A “subject” is a vertebrate, preferably a mammal, more preferably a human. The term “mammal” is used herein to refer to any animal classified as a mammal, including, without limitation, humans, domestic and farm animals, and zoo, sports, or pet animals, such as sheep, dogs, horses, cats, cows, rats, pigs, apes such as cynomolgus monkeys, to name only a few illustrative examples. Preferably, the “mammal” used herein is human.

An “effective amount” is an amount sufficient to yield beneficial or desired results. An effective amount can be administered in one or more individual administrations or doses.

As used herein, “antibody” includes full-length antibodies or any antigen binding fragment (i.e., “antigen-binding portion”) or derivatives (e.g., single chain antibody derivatives) thereof. A full-length antibody refers to a glycoprotein comprising at least two heavy chains (HCs) and two light chains (LCs) inter-connected by disulfide bonds. Each heavy chain is comprised of a heavy chain variable domain (V_{H} or HCVR) and a heavy chain constant region (C_{H}). The heavy chain constant region is comprised of three domains, C_{H1}, C_{H2} and C_{H3}. Each light chain is comprised of a light chain variable domain (V_{L} or LCVR) and a light chain constant region (G). The light chain constant region is comprised of one domain, C_{L}. The V_{H} and V_{L} regions can be further subdivided into regions of hypervariability, termed complementarity determining regions (CDRs), interspersed with regions that are more conserved, termed framework regions (FRs). Each V_{H} and V_{L} is composed of three CDRs and four FRs, arranged in the following order from the amino-terminus to the carboxy-terminus: FR1, CDR1, FR2, CDR2, FR3, CDR3, FR4. The variable regions of the heavy and light chains contain a binding domain that interacts with an antigen (for example, GPC3 or PD-L1). The constant regions of the antibodies may optionally mediate the binding of the immunoglobulin to host tissues or factors, including various cells of the immune system (e.g., effector cells) and the first component (C1q) of the classical complement system.

As used herein, “antigen binding fragment” of an antibody refers to one or more fragments of an antibody that retain the ability to specifically bind to an antigen (e.g., GPC3 or PD-L1). It has been shown that the antigen-binding function of an antibody can be performed by fragments of a full-length antibody. Examples of binding fragments encompassed within the term “antigen-binding fragment” of an antibody include (i) a Fab fragment consisting of the V_{H}, V_{L}, C_{L} and C_{H1} domains; (ii) a F(ab′)₂ fragment comprising two Fab fragments linked by a disulfide bridge at the hinge region; (iii) a Fab′ fragment consisting of the V_{H}, V_{L}, C_{L} and C_{H1} domains and the region between C_{H1} and C_{H2} domains; (iv) an Fd fragment consisting of the V_{H} and C_{H1} domains; (v) a single-chain Fv (scFv) fragment consisting of the V_{H} and V_{L} domains of a single arm of an antibody, (vi) a dAb fragment (Ward et al., 1989) consisting of a V_{H} domain; and (vii) an isolated complementarity determining region (CDR) or a combination of two or more isolated CDRs which may optionally be joined by a synthetic linker; (viii) a “diabody” comprising the V_{H} and V_{L} connected in the same polypeptide chain using a short linker (see, e.g., patent documents EP 404,097; WO 93/11161; and Holliger et al., 1993); (ix) a “domain antibody fragment” containing only the V_{H} or V_{L}, where in some instances two or more V_{H} regions are covalently joined.

As used herein a “T cell activation enhancing targeting” moiety, such as a lipocalin mutein, is a moiety that targets a receptor on a T cell, an antigen presenting cell, and/or a tumor cell or its ligand. Further, the targeting moiety is capable of stimulating, in particular co-stimulating, T cell activation, or capable of antagonizing T cell inhibition. Such a T cell activation enhancing targeting moiety can be an agonist of a co-stimulatory receptor on a T cell, also referred to herein as “T cell co-stimulatory receptor targeting” moiety. An exemplary T cell co-stimulating receptor is 4-1BB, another example is OX40. Alternatively, such a T cell activation enhancing targeting moiety can be an antagonist to an inhibitory receptor on a T cell. Antagonizing can be by binding to the inhibitory receptor or to its ligand. Co-stimulatory receptors on a T cell and inhibitory receptors on a T cell as well as their ligands are well known in the art and are, e.g., reviewed by Bakdash G dt al. (2013) Front. Immunol. 4:53 and Catakovic et al. Cell Communication and Signaling (2017) 15:1. T cell activation enhancing targeting lipocalin muteins are e.g. disclosed in WO 2006/056464, WO 2012/072806, WO 2016/177762, WO 2018/087108, WO 2017/009456, and WO 2018/134274, which are incorporated herein by reference.

As used herein, the term “tumor associated antigen (TAA)” refers to a protein or polypeptide antigen that is expressed by a tumor cell. For example, a TAA may be one or more surface proteins or polypeptides, nuclear proteins or glycoproteins, or fragments thereof, of a tumor cell. Alternatively, a TAA may refer to a protein or polypeptide antigen that is associated with the tumor stroma. TAA targeting lipocalin muteins are e.g. disclosed in WO 2009/095447, WO 2012/065978, WO 2013/174783, WO 2016/184875, WO 2012/136685, WO 2005/019256, and WO 2016/120307, which are incorporated herein by reference. Particular TAAs disclosed herein are GPC3 and PD-1.

As used herein, the term “chimeric antigen receptor” or “CAR” or “CARs” refers to an engineered receptor, which typically grafts an antigen specificity onto a receptor of a cytotoxic cell, for example T cells, NK cells and macrophages, with T cells being preferred. A CAR is an artificial fusion of multiple parts: it typically comprises at least one antigen specific targeting region (ectodomain), a transmembrane domain, and an intracellular signaling domain (endodomain) which typically contains the signaling domain(s) of one or more (co-)stimulatory immunoreceptors. An example for the ectodomain is an scFv fragment or a CD19 ligand. An example for the transmembrane domain is a CD28 transmembrane domain. An example of an endodomain is CD3-zeta. In the case of CTL019, for example, the recognition domain is an antibody single chain fragment (scFv) specific for CD19, the linker- and transmembrane regions are grafted from the membrane protein CD8, and the intracellular signaling part consists of the complete intracellular domains of 4-1BB and CD3zeta fused in tandem. When a T cell transduced with this construct encounters a CD19-positive target cell, the chimeric antigen receptor is clustered, which results in activation of the signaling pathways downstream of CD3zeta and of the co-stimulatory receptor 4-1EE, in turn leading to activation of T cell proliferation, cytokine secretion, survival and the capacity to kill. With this design, CTLO19 is an example for a “second generation” CAR, identified by the presence of two immunostimulatory domains. In contrast, “first generation” CARs contain only a single immunostimulatory domain—usually that of CD3zeta—while in “third generation” CARs, overall three intracellular immunostimulatory domains are fused in tandem, for example those of CD3zeta, CD28 and 4-1EE.

### III. DESCRIPTIONS OF FIGURES

FIG. 1: provides an overview over the design of the representative monomer polypeptides and multimeric proteins assembled thereof described in this application. Representative monomer polypeptides were generated by fusing one or more 4-1BB-targeting moieties of the disclosure (e.g., SEQ ID NOs: 56-71) to the N-terminus, C-terminus, or both N- and C-termini of an oligomerization moiety of the disclosure (e.g., SEQ ID NOs: 35-37) via linkers such as a linker shown in any one of SEQ ID NOs: 12-28. Different formats that were generated are depicted in FIGS. 1A and 1B and also include bispecific formats with one of the 4-1BB targeting moieties being replaced with an OX40-targeting moiety (FIG. 1B). Additional exemplary bispecific monomer polypeptides were generated by fusing a 4-1BB-targeting moiety of the disclosure (e.g., SEQ ID NOs: 56-71) and (1) a GPC3-targeting moiety of the disclosure (e.g., SEQ ID NOs: 74-98), (2) an OX40-targeting moiety of the disclosure (e.g., SEQ ID NOs: 174-202), or (3) an PD-L1-targeting moiety of the disclosure (e.g., SEQ ID NO: 172) to the N-terminus, C-terminus, or both N- and C-termini of an oligomerization moiety (e.g., SEQ ID NOs: 35-37) via linkers such as a linker shown in any one of SEQ ID NOs: 12-28. The different formats that were generated are depicted in FIG. 1C.

FIG. 2: shows the results of ELISA experiments in which the binding to human 4-1BB (FIG. 2A) or human GPC3 (FIG. 2B) of exemplary multimeric proteins was determined as described in Example 3. C-terminal His-tagged 4-1BB or GPC3 was coated on a microtiter plate, and the tested agents were titrated starting with the highest concentration of 100 nM. Bound agents under study were detected via anti-NGAL-HRP. The data was fit with a 1:1 binding model with the EC₅₀ value and the maximum signal as free parameters, and a slope that was fixed to one. The resulting EC₅₀ values are provided in Table 3.

FIG. 3: illustrates the results of exemplary ELISA experiments in which the ability of representative multimeric proteins to simultaneously bind GPC3 and 4-1BB, was determined as described in Example 4. Recombinant huGPC3-His was coated on a microtiter plate, followed by a titration of the multimeric proteins. Subsequently, a constant concentration of biotinylated hu4-1BB was added, which was detected via ExtrAvidin-Peroxidase. The data was fitted with a 1:1 binding model with the EC₅₀ value and the maximum signal as free parameters, and a slope that was fixed to unity. The resulting EC₅₀ values are provided in Table 4.

FIG. 4: the results of the target binding assessment of exemplary multimeric proteins by flow cytometry using human 4-1BB-expressing CHO cells (FIG. 4A), cynomolgus 4-1BB-expressing CHO cells (FIG. 4B), and human GPC3-expressing HepG2 cells (FIG. 4C), as described in Example 5. No binding was observed when using mock transfected cells (data not shown). The geometric means of the fluorescence intensity were used to calculate EC₅₀ values, which are provided in Table 5.

FIG. 5: demonstrates the potential of exemplary multimeric proteins to co-stimulate T cell activation. Mock transfected Flp-In-CHO cells were seeded into anti-CD3 antibody coated plates. Pan T cells, various concentrations of test molecules, and anti-CD28 antibody were added and incubated for three days. Levels of secreted IL-2 in the supernatant were determined by an electrochemoluminescence-based assay, as described in Example 6. Multimeric proteins that are trivalent (SEQ ID NO: 38 and SEQ ID NO: 43) did not increase IL-2 secretion. Multimeric proteins that have higher valencies than trivalency (SEQ ID NOs: 46-48 and 50-53) led to clear increase in IL-2 secretion compared to hlgG4 isotype control, with potencies comparable to the reference 4-1BB antibody (SEQ ID NOs: 72 and 73). In addition, a bispecific hexavalent protein with trivalent targeting 4-1BB and another trivalent T cell co-stimulatory receptor targeting moiety was even more potent than the reference 4-1BB antibody (SEQ ID NOs: 72 and 73).

FIG. 6: shows the ability of representative multimeric proteins to co-stimulate T cell activation. GPC3-expressing tumor cells HepG2 were seeded into anti-human CD3 coated plates. Pan T cells, various concentrations of test molecules, and anti-CD28 were added and incubated for three days. Levels of secreted IL-2 were determined, as described in Example 7. The bispecific multimeric proteins SEQ ID NO: 54 and SEQ ID NO: 55 as well as a bispecific hexavalent protein with trivalent targeting 4-1BB and another trivalent T cell co-stimulatory receptor targeting moiety lead to strong increase in IL-2 secretion, compared to the reference 4-1BB antibody SEQ ID NOs: 72 and 73. No increase of IL-2 secretion over background is observed for the reference GPC3 antibody SEQ ID NOs: 108 and 109, GPC3-specific lipocalin mutein SEQ ID NO: 90, or the 4-1BB-specific lipocalin mutein as included in the multimeric protein (SEQ ID NO: 64).

FIG. 7: shows the potential of representative multimeric proteins to activate the 4-1BB downstream signaling pathway and co-stimulate T cells, assessed using a 4-1BB bioassay as described in Example 8. NFκB-luc2/CD137 Jurkat cells were co-cultured in the absence and presence of GPC3-expressing tumor cells HepG2 with various concentrations of the multimeric proteins or controls. After 4 hours, luciferase assay reagent was added and luminescent signals were measured. Four-parameter logistic curve analysis was performed to calculate EC₅₀ values (see Table 6). The trivalent multimeric proteins SEQ ID NOs: 38-42, and 44 do not induce 4-1BB mediated T cell co-stimulation in the presence and absence of GPC3. Hexavalent multimeric proteins SEQ ID NOs: 48-53 show comparable activation in the presence and absence of GPC3. Bispecific multimeric proteins SEQ ID NO: 54 and SEQ ID NO: 55 induce GPC3-dependent 4-1BB mediated T cell co-stimulation.

FIG. 8: demonstrates the potential of exemplary multimeric proteins to co-stimulate isolated CD8+ (FIG. 8A) and isolated CD4+(FIG. 8B) T cell activation. Mock transfected Flp-In-CHO cells were seeded into anti-CD3 antibody coated plates. CD8+ or CD4+ T cells and various concentrations of test molecules were added and incubated for two days. Levels of secreted IL-2 in the supernatant were determined by an electrochemoluminescence-based assay, as described in Example 6. Multimeric protein (SEQ ID NO: 52) or reference 4-1BB antibody (SEQ ID NOs: 72 and 73) led to clear increase in IL-2 secretion by 008+ T cells compared to hlgG4 isotype control (SEQ ID NOs: 29 and 30). Multimeric protein (SEQ ID NO: 52) led to clear increase in IL-2 secretion by CD4+ T cells compared to hlgG4 isotype control (SEQ ID NOs: 29 and 30) with comparable potencies than reference 4-1BB antibody (SEQ ID NOs: 72 and 73).

FIG. 9: shows the results of the target binding assessment of exemplary multimeric proteins by flow cytometry using human 4-1BB-expressing CHO cells (FIG. 9A), human OX40-expressing CHO cells (FIG. 9B), and human PD-L1-expressing CHO cells (FIG. 9C), as described in Example 10. No binding was observed when using mock transfected cells (data not shown). The geometric means of the fluorescence intensity were used to calculate EC₅₀ values, which are provided in Table 7.

FIG. 10: shows the potential of exemplary multimeric proteins to co-stimulate T cell activation. Flp-In-CHO::huPD-L1 cells were seeded into anti-CD3 antibody coated plates. Pan T cells and various concentrations of test molecules were added and incubated for three days. Levels of secreted IL-2 in the supernatant were determined by an electrochemoluminescence-based assay, as described in Example 11. All tested multimeric proteins led to clear increase in IL-2 secretion compared to hlgG4 isotype control.

FIG. 11: shows the potential of exemplary multimeric proteins to co-stimulate isolated CD4+ T cell activation. Flp-In-CHO::huPD-L1 cells were seeded into anti-CD3 antibody coated plates. CD4+ T cells and various concentrations of test molecules were added and incubated for three days. Levels of secreted IL-2 in the supernatant were determined by an electrochemoluminescence-based assay, as described in Example 12. All tested multimeric proteins led to clear increase in IL-2 secretion by CD4+ T cells compared to hlgG4 isotype control.

FIG. 12: shows the potential of exemplary multimeric proteins to co-stimulate isolated CD8+ T cell activation. Flp-In-CHO::huPD-L1 cells were seeded into anti-CD3 antibody coated plates. CD8+ T cells and various concentrations of test molecules were added and incubated for three days. Levels of secreted IL-2 in the supernatant were determined by an electrochemoluminescence-based assay, as described in Example 13. All tested multimeric proteins led to clear increase in IL-2 secretion by CD8+ T cells compared to hlgG4 isotype control.

FIG. 13: shows the potential of representative hexavalent trimeric proteins to activate the 4-1BB downstream signaling pathway and co-stimulate T cells, assessed using a 4-1BB bioassay as described in Example 14. NFκB-luc2/CD137 Jurkat cells were co-cultured in the absence and presence of Flp-In-CHO::huOX40 cells with various concentrations of the multimeric proteins or controls. After 4 hours, luciferase assay reagent was added and luminescent signals were measured. All tested multimeric proteins led to a strong increase in 4-1BB mediated T cell co-stimulation compared to isotype controls in the presence of Flp-In-CHO::huOX40 cells but not in their absence.

FIG. 14: shows the potential of representative hexavalent trimeric proteins to activate the OX40 downstream signaling pathway and co-stimulate T cells, assessed using an OX40 bioassay as described in Example 15. NFκB-luc2/OX40 Jurkat cells were co-cultured in the absence and presence of Flp-In-CHO::hu4-1BB cells with various concentrations of the multimeric proteins or controls. After 5 hours, luciferase assay reagent was added and luminescent signals were measured. All tested multimeric proteins led to a strong increase in OX40 mediated T cell co-stimulation compared to isotype controls in the presence of Flp-In-CHO::hu4-1BB cells but not in their absence.

### IV. DETAILED DESCRIPTION OF THE DISCLOSURE

As is described herein, the present disclosure encompasses the recognition that trivalent soluble 4-1BBL may not lead to efficient 4-1BB activation, which requires higher dimension of 4-1BB clustering mediated by cell surface expression of antigen-presenting cells (Wyzgol et al., 2009, Rabu et al., 2005). Similarly, a bivalent 4-1BB-targeting molecule such as an antibody may by itself not be sufficient to induce efficient activation mediated by 4-1BB clustering. Thus, there is an unmet need for therapeutics that induce high level of 4-1BB clustering on T cells and NK cells. Accordingly, the present application provides, among other things, novel multimeric proteins, which do not contain an Fc region, for enabling the clustering of 4-1BB in an FcγR-independent manner and for inducing 4-1BB activation with high levels of 4-1BB clustering on the cell surface. The present application also provides novel approaches for clustering 4-1BB on the cell surface and stimulating 4-1BB activation and immune responses via a multimeric protein.

Additionally, 4-1BB-targeting therapeutics may be desired that do not require the crosslinking of 4-1BB-expressing cells with other cells, e.g., tumor cells. In this regard, a multimeric protein of the disclosure is capable of activating 4-1BB and co-stimulating T cells independent of the expression of an additional target protein(s).

Furthermore, a multimeric protein of the disclosure, smaller in size than antibodies, is capable of serving as a short-term acting 4-1BB agonist to reduce risks of peripheral toxicity and limitations associated with chronic 4-1BB agonism. Accordingly, a provided multimeric protein may satisfy an unmet need to provide therapeutics for disease areas, including malignancies, where current 4-1BB agonists such as monoclonal antibodies have not been able to show a convincing risk-benefit profile.

### A. Exemplary Multimeric Proteins of the Disclosure.

In some embodiments, a multimeric protein of the disclosure contains at least two monomer polypeptides, each comprising (1) a first 4-1BB-targeting moiety (T1), such as a 4-1EE-targeting lipocalin mutein, and (2) an oligomerization moiety (O), such as an oligomerization moiety shown in any one of SEQ ID NOs: 35-37. In some embodiments, a multimeric protein of the disclosure contains at least three monomer polypeptides, each comprising (1) a first 4-1BB-targeting moiety (T1), such as a 4-1BB-targeting lipocalin mutein, and (2) an oligomerization moiety (O), such as an oligomerization moiety shown in any one of SEQ ID NOs: 35-37. In some embodiments, a multimeric protein of the disclosure contains at least four monomer polypeptides, each comprising (1) a first 4-1BB-targeting moiety (T1), such as a 4-1EE-targeting lipocalin mutein, and (2) an oligomerization moiety (O), such as an oligomerization moiety shown in any one of SEQ ID NOs: 35-37. In some embodiments, a provided multimeric polypeptide contains three of such monomer polypeptides. In some embodiments, a provided multimeric polypeptide of the disclosure contains four of such monomer polypeptides.

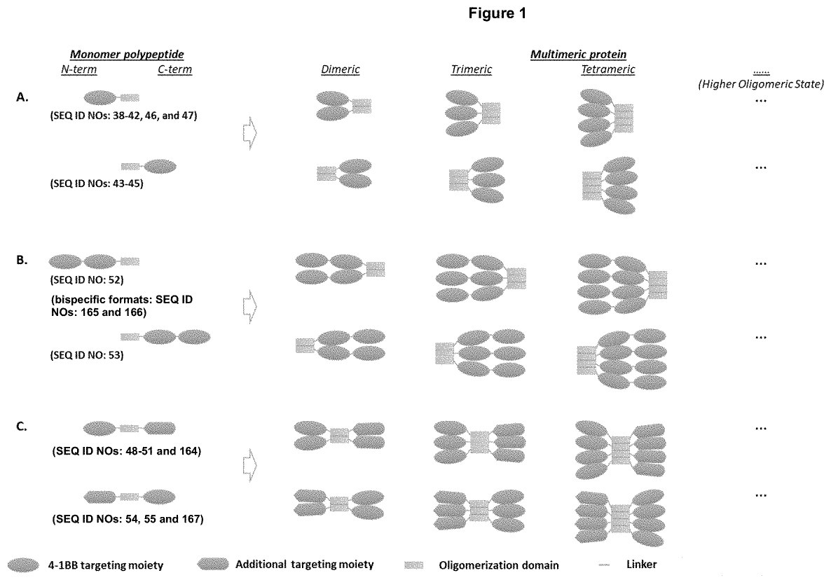

In some embodiments, the first 4-1EE-targeting moiety (T1) of a provided monomer polypeptide is fused at its N-terminus and/or its C-terminus to the oligomerization moiety (O). In some embodiments, the first 4-1BB-targeting moiety (T1) of a provided monomer polypeptide is fused to the oligomerization moiety (O) via a linker (L) (FIG. 1A). A linker as described herein may be a peptide linker, for example, as shown in any one of SEQ ID NOs: 12-28.

In some embodiments, the first 4-1EE-targeting moiety (T1) of a provided monomer polypeptide is linked at its N-terminus and/or its C-terminus to the oligomerization moiety (O). In some embodiments, the first 4-1BB-targeting moiety (T1) of a provided monomer polypeptide is linked to the oligomerization moiety (O) via a linker (L) (FIG. 1A). A linker as described herein may be a peptide linker, for example, as shown in any one of SEQ ID NOs: 12-28.

In some embodiments, the first 4-1EE-targeting moiety (T1) of a provided monomer polypeptide is linked via a linker (L), preferably a peptide linker, at its C-terminus to the N-terminus of the oligomerization moiety (O) (FIG. 1A).

In some embodiments, a monomer polypeptide of the disclosure comprises at least one additional targeting moiety (T2). In some embodiments, a monomer polypeptide comprises an additional targeting moiety (T2) which is a second 4-1BE-targeting moiety. In some embodiments, a monomer polypeptide comprises an additional moiety (T2) which is a moiety targeting another target (i.e., other than 4-1EE), e.g., a moiety that targets a tumor associated antigen, such as a GPC3- or PD-L1-targeting moiety, or a T cell activation enhancing targeting moiety (other than a 4-1BB-targeting moiety), such as an OX40-targeting moiety. Such additional targeting moiety (T2) can generally be any target specific binding molecule. Preferably, such additional targeting moiety (T2) is a lipocalin mutein, an antibody, or an antigen-binding fragment or derivative of an antibody, such as single chain variable fragment (scFv).

In some embodiments, a monomer polypeptide of the disclosure comprises an additional targeting moiety (T2), placed in tandem with the first 4-1BB-targeting moiety (T1). The additional targeting moiety (T2) and the first 4-1BB-targeting moiety (T1) may be linked via a linker, such as a peptide linker. In some particular embodiments, a monomer polypeptide comprises an additional targeting moiety (T2) that is a second 4-1BB-targeting moiety. The additional targeting moiety (T2) that is a second 4-1BB-targeting moiety may be placed in tandem with the first 4-1BB-targeting moiety (T1). The two 4-1BB-targeting moieties (T1 and T2) may be linked via a peptide linker (L) and to the N-terminus or C-terminus of the oligomerization moiety (O) (FIG. 1B). In some embodiments, where the additional targeting moiety (T2) is a second 4-1BB targeting moiety, the second 4-1BB targeting moiety may be a lipocalin mutein. The additional targeting moiety (T2) may be the same lipocalin mutein as the first 4-1BB-targeting moiety. The additional targeting moiety (T2) and the first 4-1BB-targeting moiety may be individually selected from the 4-1BB specific lipocalin muteins of the disclosure. In some embodiments, the additional targeting moiety (T2) is a moiety targeting another target (i.e., other than 4-1EE), e.g., a moiety that targets a tumor associated antigen, such as a GPC3- or PD-L1-targeting moiety, or a T cell activation enhancing targeting moiety (other than a 4-1EE-targeting moiety), such as an OX40-targeting moiety. In some particular embodiments, T1 is a 4-1BB-targeting lipocalin mutein, and T2 is an OX40-targeting lipocalin mutein.

In some embodiments, a monomer polypeptide of the disclosure has one of the following configurations (from N to C terminus) (L′ is a linker that is the same as or different from L):
a. T1-L′-T2-L-O;
b. T2-L′-T1-L-O;
c. O-L-T1-L′-T2;
d. O-L-T2-L′-T1;
e. T1-L-T2-O;
f. T2-L-T1-O;
g. O-L-T1-T2;
h. O-L-T2-T1;
i. T1-T2-L-O;
j. T2-T1-L-O;
k. O-T1-L-T2;
l. O-T2-L-T1;
m. T1-T2-O;
n. T2-T1-O;
o. O-T1-T2; or
p. O-T2-T1.

In some embodiments, a monomer polypeptide of the disclosure comprises an additional targeting moiety (T2), wherein the additional targeting moiety is linked to a different terminus of the oligomerization moiety (O) than the first 4-1BB-targeting moiety (T1). In some particular embodiments, a monomer polypeptide comprises an additional targeting moiety (T2) that is a moiety that targets a tumor associated antigen. In some particular embodiments, a monomer polypeptide comprises an additional targeting moiety (T2) that is a GPC3- or PD-L1-targeting moiety. In some particular embodiments, a monomer polypeptide comprises an additional targeting moiety (T2) that is a T cell activation enhancing targeting moiety, such as an OX40-targeting moiety. The additional targeting moiety (T2) that is preferably a moiety that targets a tumor associated antigen or a T cell activation enhancing targeting moiety may be linked to the C-terminus or N-terminus of the oligomerization moiety (O), while the first 4-11BE-targeting moiety (T1) is linked to the N-terminus or C-terminus, respectively, of the oligomerization moiety (O) (FIG. 1C). In some particular embodiments, T1 is a 4-1EE-targeting lipocalin mutein, and T2 is an OX40-targeting lipocalin mutein.

In some embodiments, a monomer polypeptide of the disclosure has one of the following configurations (from N to C terminus) (L′ is a linker that is the same as or different from L):
a. T1-L-O-L′-T2;
b. T2-L′-O-L-T1;
c. T1-L-O-T2;
d. T2-L-O-T1;
e. T1-O-L-T2;
f. T2-O-L-T1;
g. T1-O-T2; or
h. T2-O-T1.

In some embodiments, a multimeric protein of the disclosure may comprise at least four targeting moieties (T1 or T2). Such a multimeric protein may comprise at least three first 4-1EE targeting moieties (T1). As an illustrative example, the multimeric protein may comprise four first 4-1EE-targeting moieties (T1). Such an exemplary multimeric protein may comprise four monomer polypeptides that each comprise a first 4-1EE-targeting moiety (T1), and an oligomerization moiety (O), and optionally a linker (L), wherein the oligomerization domain is capable of promoting tetramerization. In another illustrative example, the multimeric protein may comprise six first 4-1EE-targeting moieties (T1). Such exemplary multimeric protein may comprise three monomer polypeptides disclosed herein, each comprising an oligomerization domain (O) that is capable of promoting trimerization and two first 4-1EE-targeting moieties (T1). In another illustrative example, the multimeric protein may comprise three first 4-1BB-targeting moieties (T1) and at least one additional targeting moiety (T2). Such a multimeric protein may have three monomer polypeptides, each comprising an oligomerization domain (O) that is capable of promoting trimerization and wherein at least one of the monomer polypeptides, preferably all monomer polypeptides, comprise an additional targeting moiety (T2). The additional targeting moieties (T2) can be any additional targeting moiety (T2) disclosed herein. Any of the monomer polypeptides may comprise one or more linkers (L) connecting the moieties T1, T2, and/or O.

In some embodiments, a multimeric protein of the disclosure may be able to bind 4-1BB with a K_{D} value of about 1 nM or lower, such as 0.94 nM or lower, about 0.68 nM or lower, about 0.5 nM or lower, about 0.3 nM or lower, or about 0.2 nM or lower. In some embodiments, a multimeric protein of the disclosure may be able to bind 4-1BB with a K_{D} value lower than the K_{D} value of the 4-1BB-targeting moiety as included in such multimeric protein, such as the lipocalin mutein shown in SEQ ID NO: 64. The K_{D} values of provided multimeric proteins may be apparent K_{D} values, for example, as described in Example 2. The K_{D} values of provided multimeric proteins may be measured, for example, in a surface-plasmon-resonance (SPR) assay, such as an SPR assay as essentially described in Example 2.

In some embodiments, a multimeric protein of the disclosure may be able to bind 4-1BB with an EC₅₀ value of about 1.5 nM or lower, such as about 0.7 nM or lower, about 0.3 nM or lower, about 0.2 nM or lower, about 0.15 nM or lower, or about 0.1 nM or lower. In some embodiments, a multimeric protein of the disclosure may be able to bind 4-1BB with an EC₅₀ value lower than the EC₅₀ value of the 4-1BB-targeting moiety as included in such multimeric protein, such as the lipocalin mutein shown in SEQ ID NO: 64. The EC₅₀ values of provided multimeric proteins may be measured, for example, in an enzyme-linked immunosorbent assay (ELISA) assay, such as an ELISA assay as essentially described in Example 3.

In some embodiments, a multimeric protein of the disclosure may be able to bind 4-1BB-expressing cells with an EC₅₀ value of about 11 nM or lower, such as about 9 nM or lower, about 7 nM or lower, about 5 nM or lower, about 4 nM or lower, about 3 nM or lower, or about 2 nM or lower. In some embodiments, a multimeric protein of the disclosure may be able to bind 4-1BB with an EC₅₀ value lower than the EC₅₀ value of the 4-1BB-targeting moiety as included in such multimeric protein, such as the lipocalin mutein shown in SEQ ID NO: 64. In some embodiments, a multimeric protein of the disclosure may be able to bind 4-1BB with an EC₅₀ value comparable to or lower than the EC₅₀ value of an anti-4-1BB antibody, such as the antibody having the heavy and light chains provided by SEQ ID NOs: 72 and 73. The EC₅₀ value of a provided multimeric protein may be measured, for example, in a flow cytometric analysis, such as a flow cytometric analysis as essentially described in Example 5. The cell expressing 4-1BB may be, for example, a CHO cell transfected with human 4-1BB.

In some embodiments, a multimeric protein of the disclosure may be cross-reactive with cynomolgus 4-1BB. In some embodiments, a provided multimeric protein may be able to bind cynomolgus 4-1BB with an EC₅₀ value of at most about 7 nM or lower, such as about 6 nM or lower, about 5 nM or lower, about 4 nM or lower, about 3 nM or lower, or about 2 nM or lower. In some embodiments, a multimeric protein of the disclosure may be able to bind cynomolgus 4-1BB with an EC₅₀ value comparable to or lower than the EC₅₀ value of an anti-4-1BB antibody, such as the antibody having the heavy and light chains provided by SEQ ID NOs: 72 and 73. The EC₅₀ value of a multimeric protein may be measured, for example, in a flow cytometric analysis, such as a flow cytometric analysis as essentially described in Example 5. The cell expressing 4-1BB may be, for example, a CHO cell transfected with cynomolgus 4-1BB.

In some embodiments, a multimeric protein of the disclosure may be able to bind GPC3-expressing cells with an EC₅₀ value of at most about 3 nM or lower, such as about 2 nM or lower, about 1 nM or lower, or about 0.5 nM or lower. In some embodiments, a multimeric protein of the disclosure may be able to bind GPC3 with an EC₅₀ value comparable to or lower than the EC₅₀ value of the anti-GPC3 antibody from which the GPC3-targeting moiety is derived, such as the antibody having the heavy and light chains provided by SEQ ID NOs: 108 and 109. The EC₅₀ value of a provided multimeric protein may be measured, for example, in a flow cytometric analysis, such as a flow cytometric analysis as essentially described in Example 5. The cells expressing GPC3 may be, for example, HepG2 cells.

In some embodiments, a multimeric protein of the disclosure may be able to simultaneously bind 4-1BB and GPC3. In some embodiments, a provided multimeric protein may be able to simultaneously bind 4-1BB and GPC3, with an EC₅₀ value of at most about 0.2 nM or lower, such as about 0.1 nM or lower. In some other embodiments, a provided multimeric protein may be able to simultaneously bind 4-1BB and GPC3, with an EC₅₀ value of at most about 1.5 nM or even lower, such as about 1.4 nM or lower, about 1.3 nM or lower, about 0.7 nM or lower, or about 0.6 nM or lower. The simultaneous binding may be determined, for example, in and ELISA assay, such as an ELISA assay as essentially described in Example 4.

In some embodiments, a multimeric protein of the disclosure may be able to bind PD-L1-expressing cells with an EC₅₀ value of at most about 10 nM or lower, such as about 9 nM or lower, about 7 nM or lower, about 5 nM or lower, about 4 nM or lower, about 3 nM or lower, about 2 nM or lower, or about 1.5 nM or lower. The EC₅₀ value of a provided multimeric protein may be measured, for example, in a flow cytometric analysis, such as a flow cytometric analysis as essentially described in Example 10. The cells expressing PD-L1 may be, for example, CHO cells transfected with human PD-L1. In some embodiments, a multimeric protein of the disclosure may be able to simultaneously bind 4-1BB and PD-L1.

In some embodiments, a multimeric protein of the disclosure may be able to bind OX40-expressing cells with an EC₅₀ value of at most about 10 nM or lower, such as about 9 nM or lower, about 7 nM or lower, about 5 nM or lower, about 4 nM or lower, about 3 nM or lower, about 2 nM or lower, about 1.5 nM or lower, about 1 nM or lower, or about 0.5 nM or lower. The EC₅₀ value of a provided multimeric protein may be measured, for example, in a flow cytometric analysis, such as a flow cytometric analysis as essentially described in Example 10. The cells expressing OX40 may be, for example, CHO cells transfected with human OX40. In some embodiments, a multimeric protein of the disclosure may be able to simultaneously bind 4-1BB and OX40.

In some embodiments, multimeric proteins of the disclosure may be able to induce increased IL-2 secretion. In some preferred embodiments, provided multimeric proteins may be able to induce a concentration-dependent IL-2 secretion and/or demonstrate a tendency to induce enhanced IL-2 secretion at higher concentrations. IL-2 secretion may be measured, for example, in a functional T cell activation assay, such as an assay as essentially described in Examples 6, 7 and/or 9. In some embodiments, the T cells are CD4+ T cells, are CD8+ T cells, or comprise both CD4+ T cells and CD8+ T cells.

In some embodiments, multimeric proteins of the disclosure may be able to co-stimulate T cell responses. In some embodiments, the T cells are CD4+ T cells, are CD8+ T cells, or comprise both. In some embodiments, multimeric proteins of the disclosure may be able to co-stimulate T cell responses in a GPC3, OX40-, 4-1BB- or PD-L1-dependent manner. In some embodiments, provided multimeric proteins are not able to co-stimulate T cell responses in the absence of GPC3, OX40, 4-1BB or PD-L1. The stimulated T cell response or T cell activation may be measured, for example, in a 4-1BB bioassay, such as an assay as essentially described in Example 8, or in an OX40 bioassay, such as an assay as essentially described in Example 15.

In some embodiments, a multimeric protein of the disclosure contains at least two, preferably three or four, monomer polypeptides, each comprising an amino acid sequence shown in any one of SEQ ID NOs: 38-55 and 164-167.

In some embodiments, a multimeric protein of the disclosure contains at least two, preferably three or four, monomer polypeptides, each comprising an amino acid sequence having at least 70%, at least 75%, at least 80%, at least 85%, at least 90%, at least 92%, at least 95%, at least 97%, at least 98%, at least 99%, or even higher sequence identity to an amino acid sequence shown in any one of SEQ ID NOs: 38-55 and 164-167.

### B. Exemplary Oligomerization Moiety as Included in the Multimeric Proteins.

In some embodiments, an oligomerization moiety comprised in a monomer polypeptide of the disclosure may convert two or more monomer polypeptides to a multimeric protein of the disclosure.

In some embodiments, an oligomerization moiety of the disclosure may be a dimerization domain, such as the GCN4 leucine zipper.

In some embodiments, an oligomerization moiety of the disclosure may be a trimerization domain, such as the C-terminal domain of T4 fibritin (foldon), trimerization domains of a collagen such as human collagen XVIII trimerization domain and human collagen XV trimerization domain, the GCN4 leucine zipper, and the trimerization motif from the lung surfactant protein. Trimerization domains of collagens have been described in the art and include trimerization domains of collagen XV, collagen XVIII, and/or collagen XXII as, e.g., described in WO 2006/048252, WO 2012/022811, WO 2012/049328, and EP 2065402). In some embodiments, a trimerization domain of the disclosure comprises an amino acid sequence having at least 70%, at least 75%, at least 80%, at least 85%, at least 90%, at least 92%, at least 95%, at least 97%, at least 98%, at least 99%, or even higher sequence identity to the amino acid sequences shown in SEQ ID NO: 35. In some embodiments, a provided trimerization domain comprises the amino acid sequence shown in SEQ ID NO: 35.

In some embodiments, an oligomerization moiety of the disclosure may be a tetramerization domain, such as the p53 tetramerization domain, GCN4 leucine zipper, and the TRP-like domain. In some embodiments, a tetramerization domain of the disclosure comprises an amino acid sequence having at least 70%, at least 75%, at least 80%, at least 85%, at least 90%, at least 92%, at least 95%, at least 97%, at least 98%, at least 99%, or even higher sequence identity to the amino acid sequences shown in any one of SEQ ID NOs: 36-37. In some embodiments, a provided tetramerization domain comprises an amino acid sequence shown in any one of SEQ ID NOs: 36-37.

### C. Exemplary Lipocalin Muteins of the Disclosure.

Lipocalins are proteinaceous binding molecules that have naturally evolved to bind ligands. Lipocalins occur in many organisms, including vertebrates, insects, plants, and bacteria. The members of the lipocalin protein family (Pervaiz and Brew, 1987) are typically small, secreted proteins and have a single polypeptide chain. They are characterized by a range of different molecular-recognition properties: their binding to various, principally hydrophobic small molecules (such as retinoids, fatty acids, cholesterols, prostaglandins, biliverdins, pheromones, tastants, and odorants), and their binding to specific cell-surface receptors and their formation of macromolecular complexes. Although they have, in the past, been classified primarily as transport proteins, it is now clear that the lipocalins fulfill a variety of physiological functions. These include roles in retinol transport, olfaction, pheromone signaling, and the synthesis of prostaglandins. Lipocalins have also been implicated in the regulation of the immune response and the mediation of cell homeostasis (reviewed, e.g., in Flower et al., 2000, Flower, 1996).

Lipocalins share unusually low levels of overall sequence conservation, often with sequence identities of less than 20%. In strong contrast, their overall folding pattern is highly conserved. The central part of the lipocalin structure consists of a single eight-stranded anti-parallel p-sheet closed back on itself to form a continuously hydrogen-bonded p-barrel. This p-barrel forms a central cavity. One end of the barrel is sterically blocked by the N-terminal peptide segment that runs across its bottom as well as three peptide loops connecting the p-strands. The other end of the p-barrel is open to the solvent and encompasses a target-binding site, which is formed by four flexible peptide loops (AB, CD, EF, and GH). It is the diversity of the loops in the otherwise rigid lipocalin scaffold that gives rise to a variety of different binding modes each capable of accommodating targets of different size, shape, and chemical character (reviewed, e.g., in Skerra, 2000, Flower et al., 2000, Flower, 1996).

A lipocalin mutein according to the present disclosure may be a mutein of any lipocalin. Examples of suitable lipocalins (also sometimes designated as “reference lipocalin,” “wild-type lipocalin,” “reference protein scaffolds,” or simply “scaffolds”) of which a mutein may be used include, but are not limited to, tear lipocalin (lipocalin-1, Tlc, or von Ebner's gland protein), retinol binding protein, neutrophil lipocalin-type prostaglandin D-synthase, p-lactoglobulin, bilin-binding protein (BBP), apolipoprotein D (APOD), neutrophil gelatinase-associated lipocalin (NGAL), a2-microglobulin-related protein (A2m), 24p3/uterocalin (24p3), von Ebner's gland protein 1 (VEGP 1), von Ebner's gland protein 2 (VEGP 2), and Major allergen Can f 1 (ALL-1). In related embodiments, a lipocalin mutein is derived from the lipocalin group consisting of human tear lipocalin (hTlc), human neutrophil gelatinase-associated lipocalin (hNGAL), human apolipoprotein D (hAPOD) and the bilin-binding protein of Pieris brassicae.

The amino acid sequence of a lipocalin mutein according to the disclosure may have a high sequence identity as compared to the reference (or wild-type) lipocalin from which it is derived, for example, hTlc or hNGAL, when compared to sequence identities with another lipocalin (see also above). In this general context the amino acid sequence of a lipocalin mutein according to the disclosure is at least substantially similar to the amino acid sequence of the corresponding reference (wild-type) lipocalin, with the proviso that there may be gaps (as defined herein) in an alignment that are the result of additions or deletions of amino acids. A respective sequence of a lipocalin mutein of the disclosure, being substantially similar to the sequences of the corresponding reference (wild-type) lipocalin, has, in some embodiments, at least 60%, at least 65%, at least 70%, at least 75%, at least 80%, at least 82%, at least 85%, at least 87%, at least 90% identity, including at least 95% identity to the sequence of the corresponding lipocalin. In this regard, a lipocalin mutein of the disclosure of course may contain substitutions as described herein which renders the lipocalin mutein capable of binding to a desired target, such as a T cell activation enhancing targeting moiety or a moiety that targets a tumor associated antigen, such as 4-1BB or GPC3.

Typically, a lipocalin mutein contains one or more mutated amino acid residues—relative to the amino acid sequence of the wild-type or reference lipocalin, for example, hTlc and hNGAL—in the four loops at the open end that comprise a ligand-binding pocket and define the entrance of ligand-binding pocket (cf. above). As explained above, these regions are essential in determining the binding specificity of a lipocalin mutein for the desired target. In some embodiments, a lipocalin mutein of the disclosure may also contain mutated amino acid residues regions outside of the four loops. In some embodiments, a lipocalin mutein of the disclosure may contain one or more mutated amino acid residues in one or more of the three peptide loops (designated BC, DE, and FG) connecting the p-strands at the closed end of the lipocalin. In some embodiments, a mutein derived from of tear lipocalin, NGAL lipocalin or a homologue thereof, may have 1, 2, 3, 4, or more mutated amino acid residues at any sequence position in the N-terminal region and/or in the three peptide loops BC, DE, and FG arranged at the end of the p-barrel structure that is located opposite to the natural lipocalin binding pocket. In some embodiments, a mutein derived from tear lipocalin, NGAL lipocalin or a homologue thereof, may have no mutated amino acid residues in peptide loop DE arranged at the end of the p-barrel structure, compared to wild-type sequence of tear lipocalin.

In some embodiments, a lipocalin mutein according to the disclosure may include one or more, such as 2, 3, 4, 5, 6, 7, 8, 9, 10, 11, 12, 13, 14, 15, 16, 17, 18, 19, 20 or even more mutated amino acid residues in comparison to the amino acid sequence of a corresponding reference (wild-type) lipocalin, provided that such a lipocalin mutein should be capable of binding to a given target, such as 4-1BB or GPC3. In some embodiments, a lipocalin mutein of the disclosure includes at least two, including 2, 3, 4, 5, or even more, mutated amino acid residues, where a native amino acid residue of the corresponding reference (wild-type) lipocalin is substituted by an arginine residue.

Any types and numbers of mutations, including substitutions, deletions, and insertions, are envisaged as long as a provided lipocalin mutein retains its capability to bind its given target, such as 4-1BB or GPC3, and/or it has a sequence identity that it is at least 60%, such as at least 65%, at least 70%, at least 75%, at least 80%, at least 85% or higher identity to the amino acid sequence of the reference (wild-type) lipocalin, for example, mature hTlc or mature hNGAL.

Specifically, in order to determine whether an amino acid residue of the amino acid sequence of a lipocalin mutein is different from a reference (wild-type) lipocalin corresponds to a certain position in the amino acid sequence of the reference (wild-type) lipocalin, a skilled artisan can use means and methods well-known in the art, e.g., alignments, either manually or by using computer programs such as BLAST2.0, which stands for Basic Local Alignment Search Tool or ClustalW or any other suitable program which is suitable to generate sequence alignments. Accordingly, the amino acid sequence of a reference (wild-type) lipocalin can serve as “subject sequence” or “reference sequence”, while the amino acid sequence of a lipocalin mutein serves as “query sequence” (see also above).

In some embodiments, a substitution is a conservative substitution. Conservative substitutions are generally the following substitutions, listed according to the amino acid to be mutated, each followed by one or more replacement(s) that can be taken to be conservative: Ala→Ser, Thr, or Val; Arg→Lys, Gln, Asn, or His; Asn→Gln, Glu, Asp, or His; Asp→Glu, Gln, Asn, or His; Gln→Asn, Asp, Glu, or His; Glu→Asp, Asn, Gln, or His; His→Arg, Lys, Asn, Gln, Asp, or Glu; Ile→Thr, Leu, Met, Phe, Val, Trp, Tyr, Ala, or Pro; Leu→Thr, Ile, Val, Met, Ala, Phe, Pro, Tyr, or Trp; Lys→Arg, His, Gln, or Asn; Met→Thr, Leu, Tyr, Ile, Phe, Val, Ala, Pro, or Trp; Phe→Thr, Met, Leu, Tyr, Ile, Pro, Trp, Val, or Ala; Ser→Thr, Ala, or Val; Thr→Ser, Ala, Val, Ile, Met, Val, Phe, Pro, or Leu; Trp→Tyr, Phe, Met, Ile, or Leu; Tyr→Trp, Phe, Ile, Leu, or Met; Val→Thr, Ile, Leu, Met, Phe, Ala, Ser, or Pro. Other substitutions are also permissible and can be determined empirically or in accord with other known conservative or non-conservative substitutions. As a further orientation, the following groups each contain amino acids that can typically be taken to define conservative substitutions for one another:
(a) Alanine (Ala), Serine (Ser), Threonine (Thr), Valine (Val)
(b) Aspartic acid (Asp), Glutamic acid (Glu), Glutamine (Gln), Asparagine (Asn), Histidine (His)
(c) Arginine (Arg), Lysine (Lys), Glutamine (Gln), Asparagine (Asn), Histidine (His)
(d) Isoleucine (Ile), Leucine (Leu), Methionine (Met), Valine (Val), Alanine (Ala), Phenylalanine (Phe), Threonine (Thr), Proline (Pro)
(e) Isoleucine (Ile), Leucine (Leu), Methionine (Met), Phenylalanine (Phe), Tyrosine (Tyr), Tryptophan (Trp)

If such conservative substitutions result in a change in biological activity, then more substantial changes, such as the following, or as further described below in reference to amino acid classes, may be introduced and the products screened for a desired characteristic. Examples of such more substantial changes are: Ala→Leu or Phe; Arg→Glu; Asn→Ile, Val, or Trp; Asp→Met; Cys→Pro; Gln→Phe; Glu→Arg; His→Gly; Ile→Lys, Glu, or Gln; Leu→Lys or Ser; Lys→Tyr; Met→Glu; Phe→Glu, Gln, or Asp; Trp→Cys; Tyr→Glu or Asp; Val→Lys, Arg, His.

In some embodiments, substantial modifications in the physical and biological properties of the lipocalin (mutein) are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain.

Naturally occurring residues are divided into groups based on common side-chain properties: (1) hydrophobic: methionine, alanine, valine, leucine, iso-leucine; (2) neutral hydrophilic: cysteine, serine, threonine, asparagine, glutamine; (3) acidic: aspartic acid, glutamic acid; (4) basic: histidine, lysine, arginine; (5) residues that influence chain orientation: glycine, proline; and (6) aromatic: tryptophan, tyrosine, phenylalanine. In some embodiments. substitutions may entail exchanging a member of one of these classes for another class.

Any cysteine residue not involved in maintaining the proper conformation of the respective lipocalin also may be substituted, generally with serine, to improve the oxidative stability of the molecule and prevent aberrant crosslinking. Conversely, cysteine bond (s) may be added to the lipocalin to improve its stability.

### D. Exemplary 4-1BB-Targeting Moiety as Included in the Multimeric Proteins.

In some embodiments, with respect to a provided multimeric protein, a 4-1BB-targeting moiety may be or comprise a 4-1BB-targeting lipocalin mutein.

As noted above, lipocalin is a polypeptide defined by its supersecondary structure, namely cylindrical β-pleated sheet supersecondary structural region comprising eight β-strands connected pair-wise by four loops at one end to define thereby a binding pocket. The present disclosure is not limited to lipocalin muteins specifically disclosed herein. In this regard, the disclosure relates to a lipocalin mutein having a cylindrical β-pleated sheet supersecondary structural region comprising eight β-strands connected pair-wise by four loops at one end to define thereby a binding pocket, wherein at least one amino acid of each of at least three of said four loops has been mutated and wherein said lipocalin is effective to bind a given target, such as 4-1BB, with detectable affinity.

In some embodiments, lipocalin muteins disclosed herein may be or comprise a mutein of mature human tear lipocalin (hTlc). A mutein of mature hTlc may be designated herein as an “hTlc mutein”. In some other embodiments, a lipocalin mutein disclosed herein is a mutein of mature human neutrophil gelatinase-associated lipocalin (hNGAL). A mutein of mature hNGAL may be designated herein as an “hNGAL mutein”.

In one aspect, the present disclosure includes any number of lipocalin muteins derived from a reference (wild-type) lipocalin, preferably derived from mature hTlc or mature hNGAL, that bind 4-1BB with detectable affinity. In a related aspect, the disclosure includes various lipocalin muteins that are capable of activating the downstream signaling pathways of 4-1BB by binding to 4-1BB. In this sense, 4-1BB can be regarded as a non-natural target of the reference (wild-type) lipocalin, preferably hTlc or hNGAL, where “non-natural target” refers to a substance that does not bind to the reference (wild-type) lipocalins under physiological conditions. By engineering reference (wild-type) lipocalins with one or more mutations at certain sequence positions, the present inventors have demonstrated that high affinity and high specificity for the non-natural target, 4-1BB, is possible. In some embodiments, at 1, 2, 3, 4, 5, 6, 7, 8, 9, 10, 11, 12 or even more nucleotide triplet(s) encoding certain sequence positions on wild-type lipocalins, a random mutagenesis may be carried out through substitution at these positions by a subset of nucleotide triplets, with the aim of generating a lipocalin mutein which is capable of binding 4-1BB.

In some embodiments, lipocalin muteins of the disclosure may have mutated, including substituted, deleted and inserted, amino acid residue(s) at one or more sequence positions corresponding to the linear polypeptide sequence of a reference lipocalin, preferably hTlc or hNGAL. In some embodiments, the number of amino acid residues of a lipocalin mutein of the disclosure that is mutated in comparison with the amino acid sequence of the reference lipocalin, preferably hTlc or hNGAL, is 1, 2, 3, 4, 5, 6, 7, 8, 9, 10, 11, 12, 13, 14, 15, 16, 17, 18, 19, 20 or more such as 25, 30, 35, 40, 45 or 50, with 1, 2, 3, 4, 5, 6, 7, 8, 9, 10, or 11 being preferred and 9, 10 or 11 being even more preferred. However, it is preferred that a lipocalin mutein of the disclosure is still capable of binding 4-1BB.

In some embodiments, a lipocalin mutein of the present disclosure may lack 1, 2, 3, 4 or more amino acids at its N-terminal end and/or 1, 2 or more amino acids at its C-terminal end, in comparison to the respective reference (wild-type) lipocalin; for example, SEQ ID NOs: 56-62. In some embodiments, the present disclosure encompasses hTlc muteins as defined above, in which the first four one, two, three, or N-terminal amino acid residues of the sequence of mature hTlc (His-His-Leu-Leu; positions 1-4) and/or the last one or two C-terminal amino acid residues (Ser-Asp; positions 157-158) of the linear polypeptide sequence of the mature hTlc have been deleted (e.g., SEQ ID NOs: 56-62). In some embodiments, the present disclosure encompasses hNGAL muteins as defined above, in which amino acid residues (Lys-Asp-Pro, positions 46-48) of the linear polypeptide sequence of the mature hNGAL have be deleted (SEQ ID NO: 67). Further, a lipocalin mutein of the disclosure may include the wild-type (natural) amino acid sequence of the reference (wild-type) lipocalin, preferably hTlc or hNGAL, outside the mutated amino acid sequence positions.

In some embodiments, one or more mutated amino acid residues incorporated into a lipocalin mutein of the disclosure does do not substantially hamper or not interfere with the binding activity to the designated target and the folding of the mutein. Such mutations, including substitution, deletion and insertion, can be accomplished at the DNA level using established standard methods (Sambrook and Russell, 2001, Molecular cloning: a laboratory manual). In some embodiments, a mutated amino acid residue(s) at one or more sequence positions corresponding to the linear polypeptide sequence of the reference (wild-type) lipocalin, preferably hTlc or hNGAL, is introduced through random mutagenesis by substituting the nucleotide triplet(s) encoding the corresponding sequence positions of the reference lipocalin with a subset of nucleotide triplets.

In some embodiments, a provided lipocalin mutein that binds 4-1BB with detectable affinity may include at least one amino acid substitution of a native cysteine residue by another amino acid, for example, a serine residue. In some embodiments, a lipocalin mutein that binds 4-1BB with detectable affinity may include one or more non-native cysteine residues substituting one or more amino acids of a reference (wild-type) lipocalin, preferably hTlc or hNGAL. In some embodiments, a lipocalin mutein according to the disclosure includes at least two amino acid substitutions of a native amino acid by a cysteine residue, hereby to form one or more cysteine bridges. In some embodiments, said cysteine bridge may connect at least two loop regions. The definition of these regions is used herein in accordance with (2000), Flower (1996) and Breustedt et al. (2005).

Generally, a lipocalin mutein of the disclosure may have about at least 70%, including at least about 80%, such as at least about 85% amino acid sequence identity, with the amino acid sequence of the mature hTlc (SEQ ID NO: 1) or mature hNGAL (SEQ ID NO: 2).

In some aspects, the present disclosure provides 4-1BB-binding hTlc muteins. In this regard, the disclosure provides one or more hTlc muteins that are capable of binding 4-1BB with an affinity measured by a K_{D} of about 300 nM, 200 nM, 150 nM, 100 nM, or lower. In some embodiments, provided hTlc muteins are capable of binding 4-1BB with an EC₅₀ value of about 250 nM, 150 nM, 100 nM, 50 nM, 20 nM, or even lower. In some other embodiments, the 4-1BB-binding hTlc muteins may be cross-reactive with cynomolgus 4-1BB (cy4-1BB).

In some embodiments, an hTlc mutein of the disclosure may interfere with the binding of 4-1BBL to 4-11BE.

In some embodiments, provided hTlc muteins may comprise a mutated amino acid residue at one or more positions corresponding to positions 5, 26-31, 33-34, 42, 46, 52, 56, 58, 60-61, 65, 71, 85, 94, 101, 104-106, 108, 111, 114, 121, 133, 148, 150, and 153 of the linear polypeptide sequence of mature hTlc (SEQ ID NO: 1).

In some embodiments, provided hTlc muteins may comprise a mutated amino acid residue at one or more positions corresponding to positions 26-34, 55-58, 60-61, 65, 104-106, and 108 of the linear polypeptide sequence of mature hTlc (SEQ ID NO: 1).

In some embodiments, provided hTlc muteins may further comprise a mutated amino acid residue at one or more positions corresponding to positions 101, 111, 114 and 153 of the linear polypeptide sequence of mature hTlc (SEQ ID NO: 1).

In some embodiments, provided hTlc muteins may comprise a mutated amino acid residue at 1, 2, 3, 4, 5, 6, 7, 8, 9, 10, 11, 12, 13, 14, 15, 16, 17, 18, 19, 20, 21, 22, 23, 24, 25, 26, or even more positions corresponding to positions 5, 26-31, 33-34, 42, 46, 52, 56, 58, 60-61, 65, 71, 85, 94, 101, 104-106, 108, 111, 114, 121, 133, 148, 150 and 153 of the linear polypeptide sequence of mature hTlc (SEQ ID NO: 1). In some preferred embodiments, the provided hTlc muteins are capable of binding 4-1BB, in particular human 4-1BB.

In some embodiments, provided hTlc muteins may comprise a mutated amino acid residue at 1, 2, 3, 4, 5, 6, 7, 8, 9, 10, 11, 12, or even more positions corresponding to positions 26-34, 55-58, 60-61, 65, 104-106 and 108 of the linear polypeptide sequence of mature hTlc (SEQ ID NO: 1). In some preferred embodiments, the provided hTlc muteins are capable of binding 4-1BB, in particular human 4-1BB.

In some embodiments, a lipocalin mutein according to the disclosure may include at least one amino acid substitution of a native cysteine residue by, e.g., a serine residue. In some embodiments, an hTlc mutein according to the disclosure includes an amino acid substitution of a native cysteine residue at positions corresponding to positions 61 and/or 153 of the linear polypeptide sequence of mature hTlc (SEQ ID NO:1) by another amino acid, such as a serine residue. In this context it is noted that it has been found that removal of the structural disulfide bond (on the level of a respective naïve nucleic acid library) of wild-type hTlc that is formed by the cysteine residues 61 and 153 (cf. Breustedt et al., 2005) may provide hTlc muteins that are not only stably folded but are also able to bind a given non-natural target with high affinity. In some embodiments, the elimination of the structural disulfide bond may provide the further advantage of allowing for the generation or deliberate introduction of non-natural disulfide bonds into muteins of the disclosure, thereby, increasing the stability of the muteins. However, hTlc muteins that bind 4-1BB and that have the disulfide bridge formed between Cys 61 and Cys 153 are also part of the present disclosure.

In some particular embodiments, an hTlc mutein of the disclosure may include one or more of the amino acid substitutions Cys 61→Ala, Phe, Lys, Arg, Thr, Asn, Gly, Gln, Asp, Asn, Leu, Tyr, Met, Ser, Pro or Trp and/or Cys 153→Ser or Ala, at positions corresponding to positions 61 and/or 153 of the linear polypeptide sequence of mature hTlc (SEQ ID NO: 1).

In some embodiments, either two or all three of the cysteine codons at positions corresponding to positions 61, 101 and 153 of the linear polypeptide sequence of mature hTlc (SEQ ID NO: 1) are replaced by a codon of another amino acid. Further, in some embodiments, an hTlc mutein according to the disclosure includes an amino acid substitution of a native cysteine residue at the position corresponding to position 101 of the linear polypeptide sequence of mature hTlc (SEQ ID NO: 1) by a serine residue or a histidine residue.

In some embodiments, a mutein according to the disclosure comprises an amino acid substitution of a native amino acid by a cysteine residue at positions corresponding to positions 28 or 105 of the linear polypeptide sequence of mature hTlc (SEQ ID NO: 1). Further, in some embodiments, a mutein according to the disclosure comprises an amino acid substitution of a native arginine residue at the position corresponding to position 111 of the linear polypeptide sequence of mature hTlc (SEQ ID NO: 1) by a proline residue. Further, in some embodiments, a mutein according to the disclosure comprises an amino acid substitution of a native lysine residue at the position corresponding to position 114 of the linear polypeptide sequence of mature hTlc (SEQ ID NO: 1) by a tryptophan residue or a glutamic acid.

In some embodiments, provided 4-1BB-binding hTlc muteins may comprise, at one or more positions corresponding to positions 5, 26-31, 33-34, 42, 46, 52, 56, 58, 60-61, 65, 71, 85, 94, 101, 104-106, 108, 111, 114, 121, 133, 148, 150, and 153 of the linear polypeptide sequence of mature hTlc (SEQ ID NO: 1), one or more of the following mutated amino acid residues: Ala 5→Val or Thr; Arg 26→Glu; Glu 27→Gly; Phe 28→Cys; Pro 29→Arg; Glu 30→Pro; Met 31→Trp; Leu 33→Ile; Glu 34→Phe; Thr 42→Ser; Gly 46→Asp; Lys 52→Glu; Leu 56→Ala; Ser 58→Asp; Arg 60→Pro; Cys 61→Ala; Lys 65→Arg or Asn; Thr 71→Ala; Val 85→Asp; Lys 94→Arg or Glu; Cys 101→Ser; Glu 104→Val; Leu 105→Cys; His 106→Asp; Lys 108→Ser; Arg 111→Pro; Lys 114→Trp; Lys 121→Glu; Ala 133→Thr; Arg 148→Ser; Ser 150→Ile; and Cys 153→Ser. In some embodiments, an hTlc mutein of the disclosure comprises two or more, such as 3, 4, 5, 6, 7, 8, 9, 10, 11, 12, 13, 14, 15, 16, 17, 18, 19, 20, 21, 22, 23, 24, 25, 26, or more, or even all mutated amino acid residues at these sequence positions of mature hTlc (SEQ ID NO: 1).

In some embodiments, provided 4-1BB-binding hTlc muteins may comprise one of the following sets of mutated amino acid residues in comparison with the linear polypeptide sequence of mature hTlc (SEQ ID NO: 1):
(a) Arg 26→Glu; Glu 27→Gly; Phe 28→Cys; Pro 29→Arg; Glu 30→Pro; Met 31→Trp; Leu 33→Ile; Glu 34→Phe; Leu 56→Ala; Ser 58→Asp; Arg 60→Pro; Cys 61→Ala; Cys 101→Ser; Glu 104→Val; Leu 105→Cys; His 106→Asp; Lys 108→Ser; Arg 111→Pro; Lys 114→Trp; and Cys 153→Ser;
(b) Ala 5→Thr; Arg 26→Glu; Glu 27→Gly; Phe 28→Cys; Pro 29→Arg; Glu 30→Pro; Met 31→Trp; Leu 33→Ile; Glu 34→Phe; Leu 56→Ala; Ser 58→Asp; Arg 60→Pro; Cys 61→Ala; Lys 65→Arg; Val 85→Asp; Cys 101→Ser; Glu 104→Val; Leu 105→Cys; His 106→Asp; Lys 108→Ser; Arg 111→Pro; Lys 114→Trp; Lys 121→Glu; Ala 133→Thr; and Cys 153→Ser;
(c) Arg 26→Glu; Glu 27→Gly; Phe 28→Cys; Pro 29→Arg; Glu 30→Pro; Met 31→Trp; Leu 33→Ile; Glu 34→Phe; Leu 56→Ala; Ser 58→Asp; Arg 60→Pro; Cys 61→Ala; Lys 65→Asn; Lys 94→Arg; Cys 101→Ser; Glu 104→Val; Leu 105→Cys; His 106→Asp; Lys 108→Ser; Arg 111→Pro; Lys 114→Trp; Lys 121→Glu; Ala 133→Thr; and Cys 153→Ser;
(d) Ala 5→Val; Arg 26→Glu; Glu 27→Gly; Phe 28→Cys; Pro 29→Arg; Glu 30→Pro; Met 31→Trp; Leu 33→Ile; Glu 34→Phe; Leu 56→Ala; Ser 58→Asp; Arg 60→Pro; Cys 61→Ala; Lys 65→Arg; Lys 94→Glu; Cys 101→Ser; Glu 104→Val; Leu 105→Cys; His 106→Asp; Lys 108→Ser; Arg 111→Pro; Lys 114→Trp; Lys 121→Glu; Ala 133→Thr; and Cys 153→Ser;
(e) Arg 26→Glu; Glu 27→Gly; Phe 28→Cys; Pro 29→Arg; Glu 30→Pro; Met 31→Trp; Leu 33→Ile; Glu 34→Phe; Thr 42→Ser; Leu 56→Ala; Ser 58→Asp; Arg 60→Pro; Cys 61→Ala; Cys 101→Ser; Glu 104→Val; Leu 105→Cys; His 106→Asp; Lys 108→Ser; Arg 111→Pro; Lys 114→Trp; Ser 150→Ile; and Cys 153→Ser;
(f) Arg 26→Glu; Glu 27→Gly; Phe 28→Cys; Pro 29→Arg; Glu 30→Pro; Met 31→Trp; Leu 33→Ile; Glu 34→Phe; Lys 52→Glu; Leu 56→Ala; Ser 58→Asp; Arg 60→Pro; Cys 61→Ala; Thr 71→Ala; Cys 101→Ser; Glu 104→Val; Leu 105→Cys; His 106→Asp; Lys 108→Ser; Arg 111→Pro; Lys 114→Trp; Ala 133→Thr; Arg 148→Ser; Ser 150→Ile; and Cys 153→Ser; and
(g) Ala 5→Thr; Arg 26→Glu; Glu 27→Gly; Phe 28→Cys; Pro 29→Arg; Glu 30→Pro; Met 31→Trp; Leu 33→Ile; Glu 34→Phe; Gly 46→Asp; Leu 56→Ala; Ser 58→Asp; Arg 60→Pro; Cys 61→Ala; Thr 71→Ala; Cys 101→Ser; Glu 104→Val; Leu 105→Cys; His 106→Asp; Lys 108→Ser; Arg 111→Pro; Lys 114→Trp; Ser 150→Ile; and Cys 153→Ser.

In some embodiments, the residual region, i.e. the region differing from positions corresponding to positions 5, 26-31, 33-34, 42, 46, 52, 56, 58, 60-61, 65, 71, 85, 94, 101, 104-106, 108, 111, 114, 121, 133, 148, 150, and 153 of the linear polypeptide sequence of mature hTlc (SEQ ID NO: 1), of an hTlc mutein of the disclosure may comprise the wild-type (natural) amino acid sequence of the linear polypeptide sequence of mature hTlc outside the mutated amino acid sequence positions.

In some embodiments, an hTlc mutein of the disclosure has at least 70% sequence identity or at least 70% sequence homology to the sequence of mature hTlc (SEQ ID NO: 1). As an illustrative example, the mutein of the SEQ ID NO: 56 has an amino acid sequence identity or a sequence homology of approximately 84% with the amino acid sequence of the mature hTlc.

In some embodiments, an hTlc mutein of the disclosure comprises an amino acid sequence as set forth in any one of SEQ ID NOs: 56-62 or a fragment or variant thereof.

In some embodiments, an hTlc mutein of the disclosure has at least 75%, at least 80%, at least 85%, at least 90%, at least 95%, at least 98%, at least 99%, or higher sequence identity to an amino acid sequence selected from the group consisting of SEQ ID NOs: 56-62.

The present disclosure also includes structural homologues of an hTlc mutein having an amino acid sequence selected from the group consisting of SEQ ID NOs: 56-62, which structural homologues have an amino acid sequence homology or sequence identity of more than about 60%, preferably more than 65%, more than 70%, more than 75%, more than 80%, more than 85%, more than 90%, more than 92% and most preferably more than 95% in relation to said hTlc mutein.

In some aspects, the present disclosure provides 4-1BB-binding hNGAL muteins. In this regard, the disclosure provides one or more hNGAL muteins that are capable of binding 4-1BB with an affinity measured by a K_{D} of about 800 nM, 700 nM, 200 nM, 140 nM, 100 nM or lower, preferably about 70 nM, 50 nM, 30 nM, 10 nM, 5 nM, 2 nM, or even lower. In some embodiments, provided hNGAL muteins are capable of binding 4-1BB with an EC₅₀ value of about 1000 nM, 500 nM, 100 nM, 80 nM, 50 nM, 25 nM, 18 nM, 15 nM, 10 nM, 5 nM, or lower.

In some embodiments, provided 4-1BB-binding hNGAL muteins may be cross-reactive with cynomolgus 4-1BB. In some embodiments, provided hNGAL muteins are capable of binding cynomolgus 4-1BB with an affinity measured by a K_{D} of about 50 nM, 20 nM, 10 nM, 5 nM, 2 nM, or even lower. In some embodiments, provided hNGAL muteins are capable of binding cynomolgus 4-1BB with an EC₅₀ value of about 100 nM, 80 nM, 50 nM, 30 nM, or even lower.

In some embodiments, an hNGAL mutein of the disclosure may interfere or compete with the binding of 4-1BBL to 4-11BE. In some other embodiments, an hNGAL mutein of the disclosure may be capable of binding 4-1BB in the presence of 4-1BBL and/or binding 4-1BBE/4-1BBL complex.

In some embodiments, provided hNGAL muteins may comprise a mutated amino acid residue at one or more positions corresponding to positions 28, 36, 40-41, 49, 52, 65, 68, 70, 72-73, 77, 79, 81, 83, 87, 94, 96, 100, 103, 106, 125, 127, 132 and 134 of the linear polypeptide sequence of mature hNGAL (SEQ ID NO: 2).

In some embodiments, provided hNGAL muteins may comprise a mutated amino acid residue at 1, 2, 3, 4, 5, 6, 7, 8, 9, 10, 11, 12, 13, 14, 15, 16, 17, 18, 19, 20, 21, or even more positions corresponding to position 28, 36, 40-41, 49, 52, 65, 68, 70, 72-73, 77, 79, 81, 83, 87, 94, 96, 100, 103, 106, 125, 127, 132, and 134 of the linear polypeptide sequence of mature hNGAL (SEQ ID NO: 2). In some preferred embodiments, the provided hNGAL muteins are capable of binding 4-1BB, in particular human 4-1BB.

In some embodiments, provided hNGAL muteins may comprise a mutated amino acid residue at one or more positions corresponding to positions 28, 36, 40-41, 49, 52, 65, 68, 70, 72-73, 77, 79, 81, 87, 96, 100, 103, 106, 125, 127, 132 and 134 of the linear polypeptide sequence of mature hNGAL (SEQ ID NO: 2) In some preferred embodiments, the provided hNGAL muteins are capable of binding 4-1BB, in particular human 4-1BB.

In some embodiments, provided hNGAL muteins may comprise a mutated amino acid residue at one or more positions corresponding to positions 36, 87, and 96 of the linear polypeptide sequence of mature hNGAL (SEQ ID NO: 2) and at one or more positions corresponding to positions 28, 40-41, 49, 52, 65, 68, 70, 72-73, 77, 79, 81, 83, 94, 100, 103, 106, 125, 127, 132, and 134 of the linear polypeptide sequence of mature hNGAL (SEQ ID NO: 2).

In other some embodiments, provided hNGAL muteins may comprise a mutated amino acid residue at one or more positions corresponding to positions 20, 25, 28, 33, 36, 40-41, 44, 49, 52, 59, 68, 70-73, 77-82, 87, 92, 96, 98, 100, 101, 103, 122, 125, 127, 132, and 134 of the linear polypeptide sequence of mature hNGAL (SEQ ID NO: 2).

In other embodiments, provided hNGAL muteins may comprise a mutated amino acid residue at one or more positions corresponding to positions 36, 40, 41, 49, 52, 68, 70, 72, 73, 77, 79, 81, 96, 100, 103, 125, 127, 132, and 134 of the linear polypeptide sequence of mature hNGAL (SEQ ID NO: 2) and at one or more positions corresponding to positions 20, 25, 33, 44, 59, 71, 78, 80, 82, 87, 92, 98, 101, and 122 of the linear polypeptide sequence of mature hNGAL (SEQ ID NO: 2).

In some embodiments, a lipocalin mutein according to the disclosure may comprise at least one amino acid substitution of a native cysteine residue by, e.g., a serine residue. In some embodiments, an hNGAL mutein according to the disclosure may comprise an amino acid substitution of a native cysteine residue at positions corresponding to positions 76 and/or 175 of the linear polypeptide sequence of mature hNGAL (SEQ ID NO: 2) by another amino acid, such as a serine residue. In this context, it is noted that it has been found that removal of the structural disulfide bond (on the level of a respective naïve nucleic acid library) of wild-type hNGAL that is formed by the cysteine residues 76 and 175 (cf. Breustedt et al., 2005) may provide hNGAL muteins that are not only stably folded but are also able to bind a given non-natural target with high affinity. In some embodiments, the elimination of the structural disulfide bond may provide the further advantage of allowing for the generation or deliberate introduction of non-natural disulfide bonds into muteins of the disclosure, thereby, increasing the stability of the muteins. However, hNGAL muteins that bind 4-1BB and that have the disulfide bridge formed between Cys 76 and Cys 175 are also part of the present disclosure.

In some embodiments, provided 4-1BB-binding hNGAL muteins may comprise, at one or more positions corresponding to positions 28, 36, 40-41, 49, 52, 65, 68, 70, 72-73, 77, 79, 81, 83, 87, 94, 96, 100, 103, 106, 125, 127, 132 and 134 of the linear polypeptide sequence of mature hNGAL (SEQ ID NO: 2), one or more of the following mutated amino acid residues: Gln 28→His; Leu 36→Gln; Ala 40→Ile; Ile 41→Arg or Lys; Gln 49→Val, Ile, His, Ser or Asn; Tyr 52→Met; Asn 65→Asp; Ser 68→Met, Ala or Gly; Leu 70→Ala, Lys, Ser or Thr; Arg 72→Asp; Lys 73→Asp; Asp 77→Met, Arg, Thr or Asn; Trp 79→Ala or Asp; Arg 81→Met, Trp or Ser; Phe 83→Leu; Cys 87→Ser; Leu 94→Phe; Asn 96→Lys; Tyr 100→Phe; Leu 103→His; Tyr 106→Ser; Lys 125→Phe; Ser 127→Phe; Tyr 132→Glu and Lys 134→Tyr. In some embodiments, an hNGAL mutein of the disclosure comprises two or more, such as 3, 4, 5, 6, 7, 8, 9, 10, 11, 12, even more such as 13, 14, 15, 16, 17, 18, 19, 20, 21, 22, 23, 24, or all mutated amino acid residues at these sequence positions of mature hNGAL (SEQ ID NO: 2).

In some embodiments, provided 4-1BB-binding hNGAL muteins may comprise, at one or more positions corresponding to positions 20, 25, 28, 33, 36, 40-41, 44, 49, 52, 59, 68, 70-73, 77-82, 87, 92, 96, 98, 100, 101, 103, 122, 125, 127, 132, and 134 of the linear polypeptide sequence of mature hNGAL (SEQ ID NO: 2), one or more of the following mutated amino acid residues: Gin 20→Arg; Asn 25→Tyr or Asp; Gln 28→His; Val 33→Ile; Leu 36→Met; Ala 40→Asn; Ile 41→Leu; Glu 44→Val or Asp; Gin 49→His; Tyr 52→Ser or Gly; Lys 59→Asn; Ser 68→Asp; Leu 70→Met; Phe 71→Leu; Arg 72→Leu; Lys 73→Asp; Asp 77→Gin or His; Tyr 78→His; Trp 79→Ile; Ile 80→Asn; Arg 81→Trp or Gin; Thr 82→Pro; Cys 87→Ser; Phe 92→Leu or Ser; Asn 96→Phe; Lys 98→Arg; Tyr 100→Asp; Pro 101→Leu; Leu 103→His or Pro; Phe 122→Tyr; Lys 125→Ser; Ser 127→Ile; Tyr 132→Trp; and Lys 134→Gly. In some embodiments, an hNGAL mutein of the disclosure comprises two or more, such as 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 mutated amino acid residues at these sequence positions of mature hNGAL (SEQ ID NO: 2).

In some embodiments, provided 4-1BB-binding hNGAL muteins may comprise, at one or more, such as 2, 3, 4, 5, 6, 7, 8, 9, 10, 11, 12, 13, 14, 15, 16, 17, 18, 19, positions corresponding to positions 36, 40, 41, 49, 52, 68, 70, 72, 73, 77, 79, 81, 96, 100, 103, 125, 127, 132, and 134 of the linear polypeptide sequence of mature hNGAL (SEQ ID NO: 2), one or more of the following mutated amino acid residues: Leu 36→Met; Ala 40→Asn; Ile 41→Leu; Gin 49→His; Tyr 52→Ser or Gly; Ser 68→Asp; Leu 70→Met; Arg 72→Leu; Lys 73→Asp; Asp 77→Gin or His; Trp 79→Ile; Arg 81→Trp or Gin; Asn 96→Phe; Tyr 100→Asp; Leu 103→His or Pro; Lys 125→Ser; Ser 127→Ile; Tyr 132→Trp; and Lys 134→Gly. In some embodiments, provided 4-1BB-binding hNGAL muteins may further comprise, at one or more, such as 2, 3, 4, 5, 6, 7, 8, 9, 10, 11, 12, 13, 14, positions corresponding to positions 20, 25, 33, 44, 59, 71, 78, 80, 82, 87, 92, 98, 101, and 122 of the linear polypeptide sequence of mature hNGAL (SEQ ID NO: 2), one or more of the following mutated amino acid residues: Gin 20→Arg; Asn 25→Tyr or Asp; Val 33→Ile; Glu 44→Val or Asp; Lys 59→Asn; Phe 71→Leu; Tyr 78→His; Ile 80→Asn; Thr 82→Pro; Phe 92→Leu or Ser; Lys 98→Arg; Pro 101→Leu; and Phe 122→Tyr.

In some embodiments, provided 4-1BB-binding hNGAL muteins may comprise one of the following sets of mutated amino acid residues in comparison with the linear polypeptide sequence of mature hNGAL (SEQ ID NO: 2):
(a) Gln 28→His; Leu 36→Gln; Ala 40→Ile; Ile 41→Lys; Gln 49→Asn; Tyr 52→Met; Ser 68→Gly; Leu 70→Thr; Arg 72→Asp; Lys 73→Asp; Asp 77→Thr; Trp 79→Ala; Arg 81→Ser; Cys 87→Ser; Asn 96→Lys; Tyr 100→Phe; Leu 103→His; Tyr 106→Ser; Lys 125→Phe; Ser 127→Phe; Tyr 132→Glu; and Lys 134→Tyr; (b) Gln 28→His; Leu 36→Gln; Ala 40→Ile; Ile 41→Arg; Gln 49→Ile; Tyr 52→Met; Asn 65→Asp; Ser 68→Met; Leu 70→Lys; Arg 72→Asp; Lys 73→Asp; Asp 77→Met; Trp 79→Asp; Arg 81→Trp; Cys 87→Ser; Asn 96→Lys; Tyr 100→Phe; Leu 103→His; Tyr 106→Ser; Lys 125→Phe; Ser 127→Phe; Tyr 132→Glu; and Lys 134→Tyr;
(c) Gln 28→His; Leu 36→Gln; Ala 40→Ile; Ile 41→Arg; Gln 49→Asn; Tyr 52→Met; Asn 65→Asp; Ser 68→Ala; Leu 70→Ala; Arg 72→Asp; Lys 73→Asp; Asp 77→Thr; Trp 79→Asp; Arg 81→Trp; Cys 87→Ser; Asn 96→Lys; Tyr 100→Phe; Leu 103→His; Tyr 106→Ser; Lys 125→Phe; Ser 127→Phe; Tyr 132→Glu; and Lys 134→Tyr;
(d) Gln 28→His; Leu 36→Gln; Ala 40→Ile; Ile 41→Lys; Gln 49→Asn; Tyr 52→Met; Asn 65→Asp; Ser 68→Ala; Leu 70→Ala; Arg 72→Asp; Lys 73→Asp; Asp 77→Thr; Trp 79→Asp; Arg 81→Trp; Cys 87→Ser; Asn 96→Lys; Tyr 100→Phe; Leu 103→His; Tyr 106→Ser; Lys 125→Phe; Ser 127→Phe; Tyr 132→Glu; and Lys 134→Tyr;
(e) Gln 28→His; Leu 36→Gln; Ala 40→Ile; Ile 41→Lys; Gln 49→Ser; Tyr 52→Met; Asn 65→Asp; Ser 68→Gly; Leu 70→Ser; Arg 72→Asp; Lys 73→Asp; Asp 77→Thr; Trp 79→Ala; Arg 81→Met; Cys 87→Ser; Asn 96→Lys; Tyr 100→Phe; Leu 103→His; Tyr 106→Ser; Lys 125→Phe; Ser 127→Phe; Tyr 132→Glu; and Lys 134→Tyr;
(f) Gln 28→His; Leu 36→Gln; Ala 40→Ile; Ile 41→Lys; Gln 49→Val; Tyr 52→Met; Asn 65→Asp; Ser 68→Gly; Leu 70→Thr; Arg 72→Asp; Lys 73→Asp; Asp 77→Arg; Trp 79→Asp; Arg 81→Ser; Cys 87→Ser; Leu 94→Phe; Asn 96→Lys; Tyr 100→Phe; Leu 103→His; Tyr 106→Ser; Lys 125→Phe; Ser 127→Phe; Tyr 132→Glu; and Lys 134→Tyr;
(g) Gln 28→His; Leu 36→Gln; Ala 40→Ile; Ile 41→Arg; Gln 49→His; Tyr 52→Met; Asn 65→Asp; Ser 68→Gly; Leu 70→Thr; Arg 72→Asp; Lys 73→Asp; Asp 77→Thr; Trp 79→Ala; Arg 81→Ser; Cys 87→Ser; Asn 96→Lys; Tyr 100→Phe; Leu 103→His; Tyr 106→Ser; Lys 125→Phe; Ser 127→Phe; Tyr 132→Glu; and Lys 134→Tyr;
(h) Gln 28→His; Leu 36→Gln; Ala 40→Ile; Ile 41→Lys; Gln 49→Asn; Tyr 52→Met; Asn 65→Asp; Ser 68→Gly; Leu 70→Thr; Arg 72→Asp; Lys 73→Asp; Asp 77→Thr; Trp 79→Ala; Arg 81→Ser; Phe 83→Leu; Cys 87→Ser; Leu 94→Phe; Asn 96→Lys; Tyr 100→Phe; Leu 103→His; Tyr 106→Ser; Lys 125→Phe; Ser 127→Phe; Tyr 132→Glu; and Lys 134→Tyr; or
(i) Gln 28→His; Leu 36→Gln; Ala 40→Ile; Ile 41→Arg; Gln 49→Ser; Tyr 52→Met; Asn 65→Asp; Ser 68→Ala; Leu 70→Thr; Arg 72→Asp; Lys 73→Asp; Asp 77→Asn; Trp 79→Ala; Arg 81→Ser; Cys 87→Ser; Asn 96→Lys; Tyr 100→Phe; Leu 103→His; Tyr 106→Ser; Lys 125→Phe; Ser 127→Phe; Tyr 132→Glu; and Lys 134→Tyr.

In some further embodiments, in the residual region, i.e. the region differing from positions 28, 36, 40-41, 49, 52, 65, 68, 70, 72-73, 77, 79, 81, 83, 87, 94, 96, 100, 103, 106, 125, 127, 132 and 134 of the linear polypeptide sequence of mature hNGAL (SEQ ID NO: 2), an hNGAL mutein of the disclosure may include the wild-type (natural) amino acid sequence of mature hNGAL outside the mutated amino acid sequence positions.

In some other embodiments, provided 4-1BB-binding hNGAL muteins may comprise one of the following sets of mutated amino acid residues in comparison with the linear polypeptide sequence of mature hNGAL (SEQ ID NO: 2):
(a) Leu 36→Met; Ala 40→Asn; Ile 41→Leu; Gln 49→His; Tyr 52→Ser; Ser 68→Asp; Leu 70→Met; Arg 72→Leu; Lys 73→Asp; Asp 77→Gln; Trp 79→Ile; Arg 81→Trp; Asn 96→Phe; Tyr 100→Asp; Leu 103→His; Lys 125→Ser; Ser 127→Ile; Tyr 132→Trp; and Lys 134→Gly;
(b) Leu 36→Met; Ala 40→Asn; Ile 41→Leu; Gln 49→His; Tyr 52→Ser; Ser 68→Asp; Leu 70→Met; Arg 72→Leu; Lys 73→Asp; Asp 77→Gln; Trp 79→Ile; Arg 81→Trp; Phe 92→Leu; Asn 96→Phe; Lys 98→Arg; Tyr 100→Asp; Pro 101→Leu; Leu 103→His; Lys 125→Ser; Ser 127→Ile; Tyr 132→Trp; and Lys 134→Gly;
(c) Asn 25→Tyr; Leu 36→Met; Ala 40→Asn; Ile 41→Leu; Gln 49→His; Tyr 52→Gly; Ser 68→Asp; Leu 70→Met; Phe 71→Leu; Arg 72→Leu; Lys 73→Asp; Asp 77→Gln; Trp 79→Ile; Arg 81→Gln; Phe 92→Ser; Asn 96→Phe; Tyr 100→Asp; Leu 103→His; Lys 125→Ser; Ser 127→Ile; Tyr 132→Trp; and Lys 134→Gly;
(d) Leu 36→Met; Ala 40→Asn; Ile 41→Leu; Gln 49→His; Tyr 52→Gly; Ser 68→Asp; Leu 70→Met; Arg 72→Leu; Lys 73→Asp; Asp 77→Gln; Tyr 78→His; Trp 79→Ile; Arg 81→Trp; Phe 92→Leu; Asn 96→Phe; Tyr 100→Asp; Leu 103→His; Lys 125→Ser; Ser 127→Ile; Tyr 132→Trp; and Lys 134→Gly;
(e) Asn 25→Asp; Leu 36→Met; Ala 40→Asn; Ile 41→Leu; Gln 49→His; Tyr 52→Gly; Ser 68→Asp; Leu 70→Met; Arg 72→Leu; Lys 73→Asp; Asp 77→Gln; Trp 79→Ile; Arg 81→Trp; Phe 92→Leu; Asn 96→Phe; Tyr 100→Asp; Leu 103→His; Lys 125→Ser; Ser 127→Ile; Tyr 132→Trp; and Lys 134→Gly;
(f) Val 33→Ile; Leu 36→Met; Ala 40→Asn; Ile 41→Leu; Gln 49→His; Tyr 52→Gly; Ser 68→Asp; Leu 70→Met; Arg 72→Leu; Lys 73→Asp; Asp 77→Gln; Trp 79→Ile; Arg 81→Trp; Phe 92→Leu; Asn 96→Phe; Tyr 100→Asp; Leu 103→His; Lys 125→Ser; Ser 127→Ile; Tyr 132→Trp; and Lys 134→Gly;
(g) Gln 20→Arg; Leu 36→Met; Ala 40→Asn; Ile 41→Leu; Glu 44→Val; Gln 49→His; Tyr 52→Gly; Ser 68→Asp; Leu 70→Met; Arg 72→Leu; Lys 73→Asp; Asp 77→Gln; Trp 79→Ile; Arg 81→Trp; Phe 92→Leu; Asn 96→Phe; Tyr 100→Asp; Leu 103→His; Phe 122→Tyr; Lys 125→Ser; Ser 127→Ile; Tyr 132→Trp; and Lys 134→Gly;
(h) Leu 36→Met; Ala 40→Asn; Ile 41→Leu; Gln 49→His; Tyr 52→Ser; Ser 68→Asp; Leu 70→Met; Arg 72→Leu; Lys 73→Asp; Asp 77→Gln; Trp 79→Ile; Ile 80→Asn; Arg 81→Trp; Thr 82→Pro; Asn 96→Phe; Tyr 100→Asp; Pro 101→Leu; Leu 103→Pro; Lys 125→Ser; Ser 127→Ile; Tyr 132→Trp; and Lys 134→Gly;
(i) Leu 36→Met; Ala 40→Asn; Ile 41→Leu; Gln 49→His; Tyr 52→Gly; Lys 59→Asn; Ser 68→Asp; Leu 70→Met; Arg 72→Leu; Lys 73→Asp; Asp 77→Gln; Trp 79→Ile; Arg 81→Trp; Phe 92→Leu; Asn 96→Phe; Tyr 100→Asp; Leu 103→His; Lys 125→Ser; Ser 127→Ile; Tyr 132→Trp; and Lys 134→Gly; and
(j) Leu 36→Met; Ala 40→Asn; Ile 41→Leu; Glu 44→Asp; Gln 49→His; Tyr 52→Ser; Ser 68→Asp; Leu 70→Met; Phe 71→Leu; Arg 72→Leu; Lys 73→Asp; Asp 77→His; Trp 79→Ile; Arg 81→Trp; Phe 92→Leu; Asn 96→Phe; Tyr 100→Asp; Leu 103→His; Lys 125→Ser; Ser 127→Ile; Tyr 132→Trp; and Lys 134→Gly.

In some embodiments, provided 4-1BB-binding hNGAL mutein may comprise the following set of mutated amino acid residues in comparison with the linear polypeptide sequence of mature hNGAL (SEQ ID NO: 2) Gln 28→His; Leu 36→Gln; Ala 40→Ile; Ile 41→Arg; Gln 49→Ile; Tyr 52→Met; Asn 65→Asp; Ser 68→Met; Leu 70→Lys; Arg 72→Asp; Lys 73→Asp; Asp 77→Met; Trp 79→Asp; Arg 81→Trp; Cys 87→Ser; Asn 96→Lys; Tyr 100→Phe; Leu 103→His; Tyr 106→Ser; Lys 125→Phe; Ser 127→Phe; Tyr 132→Glu; and Lys 134→Tyr and/or provided mutein may have at least 75%, at least 80%, at least 85%, at least 90%, at least 95%, at least 98%, at least 99%, or higher sequence identity to the amino acid sequence of SEQ ID NO: 64.

In some embodiments, in the residual region, i.e. the region differing from positions 20, 25, 28, 33, 36, 40-41, 44, 49, 52, 59, 68, 70-73, 77-82, 87, 92, 96, 98, 100, 101, 103, 122, 125, 127, 132, and 134 of the linear polypeptide sequence of mature hNGAL (SEQ ID NO: 2), of an hNGAL mutein of the disclosure may include the wild-type (natural) amino acid sequence of mature hNGAL outside the mutated amino acid sequence positions.

In some embodiments, an hNGAL mutein of the disclosure has at least 70% sequence identity or at least 70% sequence homology to the sequence of mature hNGAL (SEQ ID NO: 2). As an illustrative example, the mutein of the SEQ ID NO: 64 has an amino acid sequence identity or a sequence homology of approximately 87% with the amino acid sequence of the mature hNGAL.

In some embodiments, an hNGAL mutein of the disclosure comprises an amino acid sequence as set forth in any one of SEQ ID NOs: 63-71 or a fragment or variant thereof.

In some embodiments, an hNGAL mutein of the disclosure has at least 75%, at least 80%, at least 85%, at least 90%, at least 95%, at least 98%, at least 99%, or higher sequence identity to an amino acid sequence selected from the group consisting of SEQ ID NOs: 63-71.

The present disclosure also includes structural homologues of an hNGAL mutein having an amino acid sequence selected from the group consisting of SEQ ID NOs: 63-71, which structural homologues have an amino acid sequence homology or sequence identity of more than about 60%, preferably more than 65%, more than 70%, more than 75%, more than 80%, more than 85%, more than 90%, more than 92% and most preferably more than 95% in relation to said hNGAL mutein.

In some embodiments, the present disclosure provides a lipocalin mutein that binds 4-1BB with an affinity measured by a K_{D} of about 5 nM or lower, wherein the lipocalin mutein has at least 75%, at least 80%, at least 85%, at least 90%, at least 95%, at least 98%, at least 99%, or higher sequence identity to the amino acid sequence of SEQ ID NO: 64.

In some embodiments, a lipocalin mutein, monomer polypeptide, or multimeric protein of the present disclosure can comprise a heterologous amino acid sequence at its N- or C-Terminus, preferably C-terminus, such as a Strep II tag (SEQ ID NO: 12) or a cleavage site sequence for certain restriction enzymes, without affecting the biological activity (binding to its target, e.g., 4-1BB) of the lipocalin mutein.

In some embodiments, further modifications of a lipocalin mutein, monomer polypeptide, or multimeric protein may be introduced in order to modulate certain characteristics of the mutein, such as to improve folding stability, serum stability, protein resistance or water solubility or to reduce aggregation tendency, or to introduce new characteristics to the mutein. In some embodiments, modification(s) may result in two or more (e.g., 2, 3, 4, 5, 6, 7, 8, 9, or 10) characteristics of a provided mutein being modulated.

For example, it is possible to mutate one or more amino acid sequence positions of a lipocalin mutein, monomer polypeptide, or multimeric protein to introduce new reactive groups, for example, for the conjugation to other compounds, such as polyethylene glycol (PEG), hydroxyethyl starch (HES), biotin, peptides or proteins, or for the formation of non-naturally occurring disulphide linkages. The conjugated compound, for example, PEG and HES, can in some cases increase the serum half-life of the corresponding lipocalin mutein.

In some embodiments, a reactive group of a lipocalin mutein, monomer polypeptide, or multimeric protein may occur naturally in its amino acid sequence, such as naturally occurring cysteine residues in said amino acid sequence. In some other embodiments, such reactive group may be introduced via mutagenesis. In case a reactive group is introduced via mutagenesis, one possibility is the mutation of an amino acid at the appropriate position by a cysteine residue. Exemplary possibilities of such a mutation to introduce a cysteine residue into the amino acid sequence of an hTlc mutein include the substitutions Thr 40→Cys, Glu 73→Cys, Arg 90→Cys, Asp 95→Cys, and Glu 131→Cys of the wild-type sequence of hTlc (SEQ ID NO: 1). Exemplary possibilities of such a mutation to introduce a cysteine residue into the amino acid sequence of an hNGAL mutein include the introduction of a cysteine residue at one or more of the sequence positions that correspond to sequence positions 14, 21, 60, 84, 88, 116, 141, 145, 143, 146 or 158 of the wild-type sequence of hNGAL (SEQ ID NO: 2). The generated thiol moiety may be used to PEGylate or HESylate the mutein, monomer polypeptide, or multimeric protein, for example, in order to increase the serum half-life of a respective lipocalin mutein

In some embodiments, in order to provide suitable amino acid side chains as new reactive groups for conjugating one of the above compounds to a lipocalin mutein, artificial amino acids may be introduced to the amino acid sequence of a lipocalin mutein, monomer polypeptide, or multimeric protein. Generally, such artificial amino acids are designed to be more reactive and thus to facilitate the conjugation to the desired compound. Such artificial amino acids may be introduced by mutagenesis, for example, using an artificial tRNA is para-acetyl-phenylalanine.

In some embodiments, a lipocalin mutein, monomer polypeptide, or multimeric protein of the disclosure is fused at (at least one of) its N-terminus or its C-terminus to a protein, a protein domain or a peptide, for instance, an antibody, a signal sequence and/or an affinity tag. In some other embodiments, a lipocalin mutein of the disclosure is conjugated at its N-terminus or its C-terminus to a partner, which is a protein, a protein domain or a peptide; for instance, an antibody, a signal sequence and/or an affinity tag.

Affinity tags such as the Strep-tag or Strep-tag II (Schmidt et al., 1996), the c-myc-tag, the FLAG-tag, the His-tag or the HA-tag or proteins such as glutathione-S-transferase or combination thereof, which allow easy detection and/or purification of recombinant proteins, are examples of suitable fusion partners. As an illustrative example, a myc-His-tag, e.g. as shown in SEQ ID NO: 131, may be fused to the lipocalin mutein, monomer polypeptide, e.g. as one shown in SEQ ID NOs: 38-55, or multimeric protein, e.g. at the C-terminus. Proteins with chromogenic or fluorescent properties such as the green fluorescent protein (GFP) or the yellow fluorescent protein (YFP) are suitable fusion partners for lipocalin muteins, monomer polypeptides, or multimeric proteins of the disclosure as well. In general, it is possible to label the lipocalin mutein, monomer polypeptide, or multimeric protein of the disclosure with any appropriate chemical substance or enzyme, which directly or indirectly generates a detectable compound or signal in a chemical, physical, optical, or enzymatic reaction. For example, a fluorescent or radioactive label can be conjugated to a lipocalin mutein, monomer polypeptide, or multimeric protein to generate fluorescence or x-rays as detectable signal. Alkaline phosphatase, horseradish peroxidase and β-galactosidase are examples of enzyme labels (and at the same time optical labels) which catalyze the formation of chromogenic reaction products. In general, all labels commonly used for antibodies (except those exclusively used with the sugar moiety in the Fc part of immunoglobulins) can also be used for conjugation to the lipocalin muteins, monomer polypeptides, or multimeric proteins of the disclosure.

In some embodiments, a lipocalin mutein of the disclosure may be fused or conjugated to a moiety that extends the serum half-life of the mutein (in this regard see also International Patent Publication No. WO 2006/056464, where such strategies are described with reference to muteins of human neutrophil gelatinase-associated lipocalin (hNGAL) with binding affinity for CTLA-4). The moiety that extends the serum half-life may be a PEG molecule, a HES molecule, a fatty acid molecule, such as palmitic acid (Vajo and Duckworth, 2000), an Fc part of an immunoglobulin, a C_{H}3 domain of an immunoglobulin, a C_{H}4 domain of an immunoglobulin, an albumin binding peptide, an albumin binding protein, or a transferrin, to name only a few.

In some embodiments, if PEG is used as a conjugation partner, the PEG molecule can be substituted, unsubstituted, linear, or branched. It can also be an activated polyethylene derivative. Examples of suitable compounds are PEG molecules as described in International Patent Publication No. WO 1999/64016, in U.S. Pat. No. 6,177,074, or in U.S. Pat. No. 6,403,564 in relation to interferon, or as described for other proteins such as PEG-modified asparaginase, PEG-adenosine deaminase (PEG-ADA) or PEG-superoxide dismutase (Fuertges and Abuchowski, 1990). The molecular weight of such a polymer, such as polyethylene glycol, may range from about 300 to about 70,000 daltons, including, for example, polyethylene glycol with a molecular weight of about 10,000, of about 20,000, of about 30,000 or of about 40,000 daltons. Moreover, as e.g., described in U.S. Pat. No. 6,500,930 or 6,620,413, carbohydrate oligomers and polymers such as HES can be conjugated to a mutein of the disclosure for the purpose of serum half-life extension.

In some embodiments, if an Fc part of an immunoglobulin is used for the purpose to prolong the serum half-life of the lipocalin mutein, monomer polypeptide, or multimeric protein of the disclosure, the SynFusion™ technology, commercially available from Syntonix Pharmaceuticals, Inc. (MA, USA), may be used. The use of this Fc-fusion technology allows the creation of longer-acting biopharmaceuticals and may, for example, consist of two copies of the mutein linked to the Fc region of an antibody to improve pharmacokinetics, solubility, and production efficiency.

Examples of albumin binding peptides that can be used to extend the serum half-life of a lipocalin mutein, monomer polypeptide, or multimeric protein, are, for instance, those having a Cys-Xaa₁-Xaa₂-Xaa₃-Xaa₄-Cys consensus sequence, wherein Xaa₁ is Asp, Asn, Ser, Thr, or Trp; Xaa₂ is Asn, Gln, His, Ile, Leu, or Lys; Xaa₃ is Ala, Asp, Phe, Trp, or Tyr; and Xaa₄ is Asp, Gly, Leu, Phe, Ser, or Thr as described in U.S. Patent Publication No. 20030069395 or Dennis et al. (2002). The albumin binding protein fused or conjugated to a lipocalin mutein, monomer polypeptide, or multimeric protein to extend serum half-life may be a bacterial albumin binding protein, an antibody, an antibody fragment including domain antibodies (see U.S. Pat. No. 6,696,245, for example), or a lipocalin mutein with binding activity for albumin. Examples of bacterial albumin binding proteins include streptococcal protein G (Konig and Skerra, 1998).

In some embodiments, if the albumin-binding protein is an antibody fragment it may be a domain antibody. Domain Antibodies (dAbs) are engineered to allow precise control over biophysical properties and in vivo half-life to create the optimal safety and efficacy product profile. Domain Antibodies are for example commercially available from Domantis Ltd. (Cambridge, UK, and MA, USA).

In some embodiments, albumin itself (Osborn et al., 2002), or a biologically active fragment of albumin can be used as a partner of a lipocalin mutein of the disclosure to extend serum half-life. The term “albumin” includes all mammal albumins such as human serum albumin or bovine serum albumin or rat albumin. The albumin or fragment thereof can be recombinantly produced as described in U.S. Pat. No. 5,728,553 or European Patent Publication Nos. EP0330451 and EP0361991. Accordingly, recombinant human albumin (e.g., Recombumin® from Novozymes Delta Ltd., Nottingham, UK) can be conjugated or fused to a lipocalin mutein, monomer polypeptide, or multimeric protein of the disclosure.

In some embodiments, if a transferrin is used as a partner to extend the serum half-life of the lipocalin mutein, monomer polypeptide, or multimeric protein of the disclosure, the muteins can be genetically fused to the N or C terminus, or both, of non-glycosylated transferrin. Non-glycosylated transferrin has a half-life of 14-17 days, and a transferrin fusion protein will similarly have an extended half-life. The transferrin carrier also provides high bioavailability, biodistribution and circulating stability. This technology is commercially available from BioRexis (BioRexis Pharmaceutical Corporation, Pa., USA). Recombinant human transferrin (DeltaFerrin™) for use as a protein stabilizer/half-life extension partner is also commercially available from Novozymes Delta Ltd. (Nottingham, UK).

Yet another alternative to prolong the half-life of the lipocalin muteins of the disclosure is to fuse to the N- or C-terminus of a lipocalin mutein, monomer polypeptide, or multimeric protein a long, unstructured, flexible glycine-rich sequences (for example poly-glycine with about 20 to 80 consecutive glycine residues). This approach disclosed in International Patent Publication No. WO 2007/038619, for example, has also been term “rPEG” (recombinant PEG).

### E. Exemplary GPC3-Targeting Moiety as Included in the Multimeric Proteins.

In some embodiments, with respect to a provided multimeric protein, a GPC3-targeting moiety may be or comprise a full-length antibody or an antigen-binding domain or derivative thereof specific for GPC3. In some embodiments, a GPC3-targeting moiety may be or comprise a single chain variable fragment (scFv) specific for GPC3.

Illustrative examples of GPC3-binding antibodies of the disclosure may comprise an antigen-binding region which cross-blocks or binds to the same epitope as a GPC3-binding antibody comprising the heavy chain variable domain (V_{H}) and light chain variable domain (V_{L}) regions of a known antibody such as codrituzumab (also known as GC33 or R05137382), YP7 (including humanized YP7), HN3, and HS20. In some embodiments, a GPC3-binding antibody of the disclosure may comprise an antigen-binding region, such as any one of the three heavy chain complementarity determining regions (CDRs) (HCDR1, HCDR2 and HCDR3) and the three light chain CDRs (LCDR1, LCDR2 and LCDR3) from an antibody selected from the group consisting of codrituzumab, YP7, HN3, and HS20.

In some embodiments, a provided GPC3 antibody or antigen-binding domain or derivative thereof may have a heavy chain variable region (HCVR) selected from the group consisting of SEQ ID NOs: 104, 105, 115, 120, and 126, and/or a light chain variable region (LCVR) selected from the group consisting of SEQ ID NOs: 106, 116, 127, and 128.

In some embodiments, the heavy chain and light chain pair of a provided GPC3 antibody or antigen-binding domain or derivative thereof are or comprise a HCVR and LCVR, respectively, as follows: SEQ ID NOs: 104 and 106, SEQ ID NOs: 106 and 106, SEQ ID NOs: 115 and 116, SEQ ID NOs: 126 and 127, or SEQ ID NOs: 126 and 128.

In some embodiments, the heavy chain and light chain pair of a provided GPC3 antibody or antigen-binding domain or derivative thereof are or comprise a HCVR and LCVR, respectively, that have a sequence having at least 70%, at least 75%, at least 80%, at least 85%, at least 90%, at least 92%, at least 95%, at least 97%, at least 98%, at least 99%, or higher sequence identity to the amino acid sequences shown in SEQ ID NOs: 104 and 106, SEQ ID NOs: 106 and 106, SEQ ID NOs: 115 and 116, SEQ ID NOs: 126 and 127, or SEQ ID NOs: 126 and 128.

In some embodiments, a provided GPC3 antibody or antigen-binding domain or derivative thereof may have a heavy chain that is any one of SEQ ID NOs: 104 and 105, and/or a light chain that is SEQ ID NO: 106.

In some embodiments, the heavy chain and light chain pair of a provided GPC3 antibody are or comprise the amino acid sequences as shown in SEQ ID NOs: 104 and 106 or SEQ ID NOs: 105 and 106.

In some embodiments, the heavy chain and light chain pair of a provided GPC3 antibody are or comprise a heavy chain and a light chain that have a sequence having at least 70%, at least 75%, at least 80%, at least 85%, at least 90%, at least 92%, at least 95%, at least 97%, at least 98%, at least 99%, or higher sequence identity to the amino acid sequences as shown in SEQ ID NOs: 104 and 106 or SEQ ID NOs: 105 and 106.

In some embodiments, a provided GPC3 antibody or antigen-binding domain thereof may have a HCVR with at least 70%, at least 75%, at least 80%, at least 85%, at least 90%, at least 92%, at least 95%, at least 97%, at least 98%, at least 99%, or even higher sequence identity to an amino acid sequence selected from the group consisting of SEQ ID NOs: 104, 105, 115, 120, and 126, and/or a LCVR with at least 70%, at least 75%, at least 80%, at least 85%, at least 90%, at least 92%, at least 95%, at least 97%, at least 98%, at least 99%, or even higher sequence identity to an amino acid sequence selected from the group consisting of SEQ ID NOs: 106, 116, 127, and 128. In other embodiments, a provided GPC3 antibody or antigen-binding domain thereof may have a heavy chain with at least 70%, at least 75%, at least 80%, at least 85%, at least 90%, at least 92%, at least 95%, at least 97%, at least 98%, at least 99%, or even higher sequence identity to an amino acid sequence selected from the group consisting of SEQ ID NOs: 104 and 105, and/or a light chain with at least 70%, at least 75%, at least 80%, at least 85%, at least 90%, at least 92%, at least 95%, at least 97%, at least 98%, at least 99%, or even higher sequence identity to the amino acid sequence of SEQ ID NO: 106.

In some embodiments, the heavy chain variable region of a provided GPC3 antibody or antigen-binding domain thereof may have the three CDRs having following sequences: GYTFTDYE (HCDR1, SEQ ID NO: 99), LDPKTGDT (HCDR2, SEQ ID NO: 100), TRFYSYTY (HCDR3; SEQ ID NO: 101). In some embodiments, the heavy chain variable region of a provided GPC3 antibody or antigen-binding domain thereof may have the three CDRs having following sequences: GFTFNKNA (HCDR1, SEQ ID NO: 110), IRNKTNNYAT (HCDR2, SEQ ID NO: 111), VAGNSFAY (HCDR3; SEQ ID NO: 112). In some embodiments, the heavy chain variable region of a provided GPC3 antibody or antigen-binding domain thereof may have the three CDRs having following sequences: YFDFDSYE (HCDR1, SEQ ID NO: 117), IYHSGST (HCDR2, SEQ ID NO: 118), ARVNMDRFDY (HCDR3; SEQ ID NO: 119). In some embodiments, the heavy chain variable region of a provided GPC3 antibody or antigen-binding domain thereof may have the three CDRs having following sequences: GFTFSSYA (HCDR1, SEQ ID NO: 122), IQKQGLPT (HCDR2, SEQ ID NO: 122), AKNRAKFDY (HCDR3; SEQ ID NO: 123).

In some embodiments the light chain variable region of a provided GPC3 antibody or antigen-binding domain thereof may have the three CDRs having following sequences: QSLVHSNRNTY (LCDR1, SEQ ID NO: 102), KVS (LCDR2), SQNTHVPPT (LCDR3; SEQ ID NO: 103). In some embodiments the light chain variable region of a provided GPC3 antibody or antigen-binding domain thereof may have the three CDRs having following sequences: QSLLYSSNQKNY (LCDR1, SEQ ID NO: 113), WAS (LCDR2), QQYYNYPLT (LCDR3; SEQ ID NO: 114). In some embodiments the light chain variable region of a provided GPC3 antibody or antigen-binding domain thereof may have the three CDRs having following sequences: QSISSY (LCDR1, SEQ ID NO: 124), NAS (LCDR2), QQNRGFPLT (LCDR3; SEQ ID NO: 125).

In some embodiments, a provided GPC3 antibody or antigen-binding domain thereof comprises a heavy chain variably region that has the three CDRs having following sequences: GYTFTDYE (HCDR1, SEQ ID NO: 99), LDPKTGDT (HCDR2, SEQ ID NO: 100), TRFYSYTY (HCDR3; SEQ ID NO: 101), and a light chain variably region that has the three CDRs having following sequences: QSLVHSNRNTY (LCDR1, SEQ ID NO: 102), KVS (LCDR2), SQNTHVPPT (LCDR3; SEQ ID NO: 103). In some embodiments, a provided GPC3 antibody or antigen-binding domain thereof comprises a heavy chain variably region that has the three CDRs having following sequences: GFTFNKNA (HCDR1, SEQ ID NO: 110), IRNKTNNYAT (HCDR2, SEQ ID NO: 111), VAGNSFAY (HCDR3; SEQ ID NO: 112), and a light chain variably region that has the three CDRs having following sequences: QSLLYSSNQKNY (LCDR1, SEQ ID NO: 113), WAS (LCDR2), QQYYNYPLT (LCDR3; SEQ ID NO: 114). In some embodiments, a provided GPC3 antibody or antigen-binding domain thereof comprises a heavy chain variably region that has the three CDRs having following sequences: GFTFSSYA (HCDR1, SEQ ID NO: 121), IQKQGLPT (HCDR2, SEQ ID NO: 122), AKNRAKFDY (HCDR3; SEQ ID NO: 123), and a light chain variably region that has the three CDRs having following sequences: QSISSY (LCDR1, SEQ ID NO: 124), NAS (LCDR2), QQNRGFPLT (LCDR3; SEQ ID NO: 125).

In some embodiments, a single chain variable fragment (scFv) specific for GPC3 disclosed herein may be derived from a GPC3 antibody having the amino acid sequences as shown in SEQ ID NOs: 104 and 106 or SEQ ID NOs: 105 and 106. In some embodiments, a provided scFv specific for GPC3 may have at least 70%, at least 75%, at least 80%, at least 85%, at least 90%, at least 92%, at least 95%, at least 97%, at least 98%, at least 99%, or even higher sequence identity to the amino acid sequence shown in SEQ ID NO: 98. In some embodiments, a provided scFv specific for GPC3 may comprise the amino acid sequence shown in SEQ ID NO: 98.

Unless otherwise indicated, all CDR sequences disclosed herein are defined according to the IMGT method as described in Lefranc, M.-P., The Immunologist, 7, 132-136 (1999). CDR1 consists of positions 27 to 38, CDR2 consists of positions 56 to 65, CDR3 for germline V-genes consists of positions 105 to 116, CDR3 for rearranged V-J-genes or V-D-J-genes consists of positions 105 to 117 (position preceding J-PHE or J-TRP 118) with gaps at the top of the loop for rearranged CDR3-IMGT with less than 13 amino acids, or with additional positions 112.1, 111.1, 112.2, 111.2, etc. for rearranged CDR3-IMGT with more than 13 amino acids. The positions given in this paragraph are according to the IMGT numbering described in Lefranc, M.-P., The Immunologist, 7, 132-136 (1999).

Various techniques for the production of antibodies or antigen-binding domains or derivatives thereof are well known in the art and described, e.g., in Altshuler et al. (2010). Thus, for example, polyclonal antibodies can be obtained from the blood of an animal following immunization with an antigen in mixture with additives and adjuvants and monoclonal antibodies can be produced by any technique which provides antibodies produced by continuous cell line cultures. Examples of such techniques are described, e.g., Harlow and Lane (1999), (1988), and include the hybridoma technique originally described by Köhler and Milstein, 1975, the trioma technique, the human B cell hybridoma technique (see e.g. Li et al., 2006, Kozbor and Roder, 1983) and the EBV-hybridoma technique to produce human monoclonal antibodies (Cole et al., 1984). Furthermore, recombinant antibodies may be obtained from monoclonal antibodies or can be prepared de novo using various display methods such as phage, ribosomal, mRNA, or cell display. In some embodiments, a suitable system for the expression of the recombinant (humanized) antibodies or fragments thereof may be selected from, for example, bacteria, yeast, insects, mammalian cell lines or transgenic animals or plants (see, e.g., U.S. Pat. No. 6,080,560; Holliger and Hudson, 2005). Further, techniques described for the production of single chain antibodies (see, inter alia, U.S. Pat. No. 4,946,778) can be adapted to produce single chain antibodies specific for the target of this invention. Surface plasmon resonance as employed in the BIAcore system can be used to increase the efficiency of phage antibodies.

In some other embodiments, with respect to a provided multimeric protein, a GPC3-targeting moiety may be or comprise a GPC3-targeting lipocalin mutein.

In some aspects, the present disclosure provides GPC3-binding hNGAL muteins. In this regard, the disclosure provides one or more hNGAL muteins that are capable of binding GPC3 with an affinity measured by a K_{D} of about 1 nM, 0.5 nM, 0.3 nM, 0.2 nM, or lower.

In some embodiments, provided GPC3-binding hNGAL muteins may comprise a mutated amino acid residue at one or more positions corresponding to positions 36, 40, 41, 49, 52, 65, 68, 70, 72, 73, 77, 79, 81, 87, 96, 100, 103,105, 106, 125, 127, 132, 134, 136, and 175 of the linear polypeptide sequence of mature hNGAL (SEQ ID NO: 2).

In some embodiments, provided GPC3-binding hNGAL muteins may comprise, at one or more positions corresponding to positions 36, 40, 41, 49, 52, 65, 68, 70, 72, 73, 77, 79, 81, 87, 96, 100, 103, 105, 106, 125, 127, 132, 134, 136, and 175 of the linear polypeptide sequence of mature hNGAL (SEQ ID NO: 2), one or more of the following mutated amino acid residues: Leu 36→Val or Arg.; Ala 40→Leu, Val or Gly; Ile 41→Leu, Arg, Met, Gly or Ala; Gln 49→Pro or Leu; Tyr 52→Arg or Trp; Asn 65→Asp; Ser 68→Val, Gly, Asn or Ala; Leu 70→Arg, Ser, Ala or Val; Arg 72→Asp, Trp, Ala, or Gly; Lys 73→Gly, Arg, Asn, Glu or Ser; Cys 76→Val or Ile; Asp 77→His, Met, Val, Leu, Thr or Lys; Trp 79→Lys, Ser or Thr; Arg 81→Gly; Cys 87→Ser; Asn 96→Arg, Asp, Gln or Pro; Tyr 100→Gly, Glu, Pro or Gln; Leu 103→Glu, Gln, Asn, Gly, Ser, Asp, or Tyr; Ser 105→Ala; Tyr 106→Asn, Ser or Thr; Lys 125→Glu; Ser 127→Arg or Tyr; Tyr 132→Trp or Ile; Lys 134→Ala or Phe; Thr 136→Ile; and Cys 175→Ala. In some embodiments, an hNGAL mutein of the disclosure comprises two or more, such as 3, 4, 5, 6, 7, 8, 9, 10, 11, 12, even more such as 13, 14, 15, 16, 17, 18, 19, 20, 21, 22, 23, 24, or all mutated amino acid residues at these sequence positions of mature hNGAL (SEQ ID NO: 2).

In some embodiments, provided GPC3-binding hNGAL muteins may comprise one of the following sets of mutated amino acid residues in comparison with the linear polypeptide sequence of mature hNGAL (SEQ ID NO: 2):
(a) Leu 36→Val; Ile 41→Leu; Gln 49→Leu; Tyr 52→Arg; Asn 65→Asp; Ser 68→Val; Leu 70→Ser; Arg 72→Trp; Lys 73→Arg; Asp 77→His; Trp 79→Lys; Arg 81→Gly; Cys 87→Ser; Asn 96→Asp; Tyr 100→Gly; Leu 103→Gln; Tyr 106→Asn; Lys 125→Glu; Ser 127→Arg; Tyr 132→Trp; and Lys 134→Ala;
(b) Leu 36→Val; Ala 40→Val; Ile 41→Arg; Gln 49→Pro; Tyr 52→Arg; Asn 65→Asp; Ser 68→Gly; Leu 70→Ser; Lys 73→Gly; Asp 77→His; Trp 79→Lys; Arg 81→Gly; Cys 87→Ser; Asn 96→Asp; Tyr 100→Gly; Leu 103→Glu; Tyr 106→Asn; Lys 125→Glu; Ser 127→Arg; Tyr 132→Trp; and Lys 134→Phe;
(c) Leu 36→Val; Ala 40→Gly; Ile 41→Met; Gln 49→Leu; Tyr 52→Arg; Asn 65→Asp; Leu 70→Ala; Lys 73→Asn; Asp 77→His; Trp 79→Lys; Arg 81→Gly; Cys 87→Ser; Asn 96→Gln; Tyr 100→Gly; Leu 103→Glu; Tyr 106→Asn; Lys 125→Glu; Ser 127→Arg; Tyr 132→Trp; and Lys 134→Phe;
(d) Leu 36→Arg; Ala 40→Val; Ile 41→Gly; Gln 49→Pro; Tyr 52→Trp; Asn 65→Asp; Ser 68→Asn; Leu 70→Arg; Arg 72→Ala; Lys 73→Arg; Asp 77→Leu; Trp 79→Ser; Arg 81→Gly; Cys 87→Ser; Asn 96→Gln; Tyr 100→Glu; Leu 103→Asn; Ser 105→Ala; Tyr 106→Asn; Lys 125→Glu; Ser 127→Tyr; Tyr 132→Ile; Lys 134→Phe; and Thr 136→Ile;
(e) Leu 36→Arg; Ala 40→Val; Ile 41→Gly; Gln 49→Pro; Tyr 52→Trp; Asn 65→Asp; Ser 68→Asn; Leu 70→Arg; Arg 72→Ala; Lys 73→Arg; Asp 77→Thr; Trp 79→Ser; Arg 81→Gly; Cys 87→Ser; Asn 96→Gln; Tyr 100→Glu; Leu 103→Gly; Ser 105→Ala; Tyr 106→Asn; Lys 125→Glu; Ser 127→Tyr; Tyr 132→Ile; Lys 134→Phe; and Thr 136→Ile;
(f) Leu 36→Arg; Ala 40→Gly; Ile 41→Ala; Gln 49→Pro; Tyr 52→Trp; Asn 65→Asp; Ser 68→Asn; Leu 70→Arg; Arg 72→Ala; Lys 73→Arg; Asp 77→Val; Trp 79→Ser; Arg 81→Gly; Cys 87→Ser; Asn 96→Pro; Tyr 100→Glu; Leu 103→Asn; Ser 105→Ala; Tyr 106→Ser; Lys 125→Glu; Ser 127→Tyr; Tyr 132→Ile; Lys 134→Phe; and Thr 136→Ile;
(g) Leu 36→Arg; Ala 40→Val; Ile 41→Ala; Gln 49→Pro; Tyr 52→Arg; Asn 65→Asp; Ser 68→Ala; Leu 70→Arg; Arg 72→Ala; Lys 73→Arg; Asp 77→Leu; Trp 79→Ser; Arg 81→Gly; Cys 87→Ser; Asn 96→Arg; Tyr 100→Glu; Leu 103→Tyr; Ser 105→Ala; Tyr 106→Asn; Lys 125→Glu; Ser 127→Tyr; Tyr 132→Ile; Lys 134→Phe; and Thr 136→Ile;
(h) Leu 36→Arg; Ala 40→Val; Ile 41→Ala; Gln 49→Pro; Tyr 52→Arg; Asn 65→Asp; Ser 68→Asn; Leu 70→Val; Arg 72→Ala; Lys 73→Gly; Asp 77→Lys; Trp 79→Ser; Arg 81→Gly; Cys 87→Ser; Asn 96→Arg; Tyr 100→Pro; Leu 103→Asn; Ser 105→Ala; Tyr 106→Asn; Lys 125→Glu; Ser 127→Tyr; Tyr 132→Ile; Lys 134→Phe; and Thr 136→Ile;
(i) Leu 36→Arg; Ala 40→Leu; Ile 41→Gly; Gln 49→Pro; Tyr 52→Trp; Asn 65→Asp; Ser 68→Asn; Leu 70→Arg; Arg 72→Ala; Lys 73→Arg; Asp 77→Met; Trp 79→Ser; Arg 81→Gly; Cys 87→Ser; Asn 96→Gln; Tyr 100→Glu; Leu 103→Ser; Ser 105→Ala; Tyr 106→Asn; Lys 125→Glu; Ser 127→Tyr; Tyr 132→Ile; and Lys 134→Phe;
(j) Leu 36→Arg; Ala 40→Val; Ile 41→Gly; Gln 49→Pro; Tyr 52→Trp; Asn 65→Asp; Ser 68→Asn; Leu 70→Arg; Arg 72→Ala; Lys 73→Gly; Cys 76→Val; Asp 77→Lys; Trp 79→Thr; Arg 81→Gly; Cys 87→Ser; Asn 96→Gln; Tyr 100→Glu; Leu 103→Asn; Ser 105→Ala; Tyr 106→Thr; Lys 125→Glu; Ser 127→Tyr; Tyr 132→Ile; Lys 134→Phe; and Cys 175→Ala;
(k) Leu 36→Arg; Ala 40→Val; Ile 41→Gly; Gln 49→Pro; Tyr 52→Arg; Asn 65→Asp; Ser 68→Gly; Leu 70→Arg; Arg 72→Gly; Lys 73→Glu; Cys 76→Ile; Asp 77 Lys; Trp 79→Ser; Arg 81→Gly; Cys 87→Ser; Asn 96→Gln; Tyr 100→Gln; Leu 103→Asp; Ser 105→Ala; Tyr 106→Thr; Lys 125→Glu; Ser 127→Tyr; Tyr 132 Ile; Lys 134→Phe; Thr 136→Ile; and Cys 175→Ala; and
(1) Leu 36→Arg; Ala 40→Val; Ile 41→Gly; Gln 49→Pro; Tyr 52→Arg; Asn 65→Asp; Ser 68→Gly; Leu 70→Arg; Arg 72→Asp; Lys 73→Ser; Cys 76→Val; Asp 77→Thr; Trp 79→Ser; Arg 81→Gly; Cys 87→Ser; Asn 96→Gln; Tyr 100→Glu; Leu 103→Asn; Ser 105→Ala; Tyr 106→Thr; Lys 125→Glu; Ser 127→Tyr; Tyr 132→Ile; Lys 134→Phe; Thr 136→Ile; Cys 175→Ala.

In some embodiments, in the residual region, i.e. the region differing from positions 36, 40, 41, 49, 52, 65, 68, 70, 72, 73, 77, 79, 81, 87, 96, 100, 103, 105, 106, 125, 127, 132, 134, 136, and 175 of the linear polypeptide sequence of mature hNGAL (SEQ ID NO: 2), of a GPC3-binding hNGAL mutein of the disclosure may include the wild-type (natural) amino acid sequence of mature hNGAL outside the mutated amino acid sequence positions.

In some embodiments, a GPC3-binding hNGAL mutein of the disclosure has at least 70% sequence identity or at least 70% sequence homology to the sequence of mature hNGAL (SEQ ID NO: 2). As an illustrative example, the mutein of the SEQ ID NO: 90 has an amino acid sequence identity or a sequence homology of approximately 87% with the amino acid sequence of the mature hNGAL.

In some embodiments, a GPC3-binding hNGAL mutein of the disclosure comprises an amino acid sequence as set forth in any one of SEQ ID NOs: 74-97 or a fragment or variant thereof.

In some embodiments, a GPC3-binding hNGAL mutein of the disclosure has at least 75%, at least 80%, at least 85%, at least 90%, at least 95%, at least 98%, at least 99%, or higher sequence identity to an amino acid sequence selected from the group consisting of SEQ ID NOs: 74-97.

The present disclosure also includes structural homologues of a GPC3-binding hNGAL mutein having an amino acid sequence selected from the group consisting of SEQ ID NOs: 74-97, which structural homologues have an amino acid sequence homology or sequence identity of more than about 60%, preferably more than 65%, more than 70%, more than 75%, more than 80%, more than 85%, more than 90%, more than 92% and most preferably more than 95% in relation to said hNGAL mutein.

### F. Exemplary PD-L1-Targeting Moiety as Included in the Multimeric Proteins.

In some embodiments, with respect to a provided multimeric protein, a PD-L1-targeting moiety may be or comprise a single chain variable fragment (scFv) specific for PD-L1. For example, an scFv specific for PD-L1 may be derived from a PD-L1-specific antibody selected from the group consisting of atezolizumab (also known as MPDL3280A or RG7446, trade name Tecentriq®), avelumab (also known as MSB0010718C, trade name Bavencio®), durvalumab (previously known as MED14736, trade name Imfinzi®), and BMS-936559 (also known as MDX-1105). These and other suitable PD-L1-specific antibodies are further described, e.g., in WO 2020/025659 A1, which is herein incorporated by reference in its entirety.

In some embodiments, a provided scFv specific for PD-L1 may have at least 70%, at least 75%, at least 80%, at least 85%, at least 90%, at least 92%, at least 95%, at least 97%, at least 98%, at least 99%, or even higher sequence identity to the amino acid sequence shown in SEQ ID NO: 172. In some embodiments, a provided scFv specific for PD-L1 comprises heavy chain CDRs (HCDR1, HCDR2 and HCDR3) and light chain CDRs (LCDR1, LCDR2 and LCDR3) which have the same sequence as the heavy chain CDRs and light chain CDRs of SEQ ID NO: 172. In some embodiments, a provided scFv specific for PD-L1 may comprise the amino acid sequence shown in SEQ ID NO: 172.

### G. Exemplary OX40-Targeting Moiety as Included in the Multimeric Proteins.

In some embodiments, with respect to a provided multimeric protein, an OX40-targeting moiety may be or comprise an OX40-targeting lipocalin mutein.

In some embodiments, the OX40-targeting lipocalin mutein is an hTlc mutein. In some other embodiments, the OX40-targeting lipocalin mutein is an hNGAL mutein. In some embodiments, the mutein is capable of binding OX40 with an affinity measured by K_{D} of about 500 nM or lower, about 400 nM or lower, about 300 nM or lower, about 200 nM or lower, about 150 nM or lower, about 100 nM or lower, about 70 nM or lower, about 50 nM or lower, about 30 nM or lower, about 20 nM or lower, about 15 nM or lower, about 10 nM or lower, about 5 nM or lower, about 3 nM or lower, about 2 nM or lower, about 1 nM or lower, about 0.5 nM or even lower, as determined, e.g., in a surface-plasmon-resonance (SPR) assay. In some embodiments, the mutein binds OX40 with an EC₅₀ value of about 250 nM or lower, about 200 nM or lower, about 150 nM or lower, about 100 nM or lower, about 70 nM or lower, about 50 nM or lower, about 30 nM or lower, about 20 nM or lower, about 15 nM or lower, about 10 nM or lower, about 7 nM or lower, about 5 nM or lower, about 3 nM or lower, about 2 nM or lower, about 1 nM or even lower, as determined, e.g., in a fluorescence activated cell sorting (FACS) assay. In some embodiments, the mutein is cross-reactive with both human OX40 and cynomolgus OX40. In some embodiments, the mutein interferes with the binding of OX40 ligand (OX40L) to OX40. In some embodiments, the mutein competes with OX40L for binding to OX40.

In some embodiments, provided OX40-binding hTlc muteins may comprise a mutated amino acid residue at one or more (e.g., at 1, 2, 3, 4, 5, 6, 7, 8, 9, 10, 11, 12, 13, 14, 15, 16, 17, 18, 19, 20, 21, 22, 23, 24, 25, 26, 27, 28, or more) positions corresponding to positions 5, 6, 8, 11, 19, 23, 26-34, 36, 37, 40, 52, 55-56, 58, 60-61, 65, 79, 86, 101, 104-106, 108, 111, 113-114, 116, 121, 124, 137, 140, 148, and 153 of the linear polypeptide sequence of mature hTlc (SEQ ID NO: 1). In some embodiments, an hTlc mutein of the disclosure comprises 10 or more mutated amino acid residues at one or more of the above-mentioned positions of the linear polypeptide sequence of mature hTlc (SEQ ID NO: 1). In some embodiments, an hTlc mutein of the disclosure comprises 15 or more mutated amino acid residues at one or more of the above-mentioned positions of the linear polypeptide sequence of mature hTlc (SEQ ID NO: 1). In some embodiments, an hTlc mutein of the disclosure comprises 20 or more mutated amino acid residues at one or more of the above-mentioned positions of the linear polypeptide sequence of mature hTlc (SEQ ID NO: 1).

In some embodiments, provided OX40-binding hTlc muteins may comprise, at one or more positions corresponding to positions 5, 6, 8, 11, 19, 23, 26-34, 36, 37, 40, 52, 55-56, 58, 60-61, 65, 79, 86, 101, 104-106, 108, 111, 113-114, 116, 121, 124, 137, 140, 148, and 153 of the linear polypeptide sequence of mature hTlc (SEQ ID NO: 1), one or more of the following mutated amino acid residues: Ala 5→Thr; Ser 6→Thr; Glu 8→Lys; Gln 11→Arg; Leu 19→Met or Gln; Thr 23→Lys; Arg 26→Trp; Glu 27→Asp; Phe 28→Cys; Pro 29→Asn; Glu 30→Gln; Met 31→Pro; Asn 32→Ile; Leu 33→Phe; Glu 34→Asp; Val 36→Asp; Thr 37→Ala; Thr 40→Ile; Lys 52→Glu; Met 55→Ile; Leu 56→Phe; Ser 58→Asp; Arg 60→Lys; Cys 61→Tyr; Lys 65→Ile; Ala 79→Thr; Ala 86→Thr; Cys 101→Ser; Glu 104→Gln; Leu 105→Cys; His 106→Pro; Lys 108→Ile; Arg 111→Pro; Val 113→Met or Leu; Lys 114→Trp; Val 116→Ala; Lys 121→Met; Leu 124→Lys; Arg 137→His; Ser 140→Arg; Arg 148→Ser or Trp; and Cys 153→Ser. In some embodiments, an hTlc mutein of the disclosure comprises two or more, such as 3, 4, 5, 6, 7, 8, 9, 10, 11, 12, 13, 14, 15, 16, 17, 18, 19, 20, 21, 22, 23, 24, 25, 26, 27, 28 or more, or even all of the above-mentioned mutated amino acid residues at these sequence positions of mature hTlc (SEQ ID NO: 1). In some embodiments, an hTlc mutein of the disclosure comprises 10 or more of the above-mentioned mutated amino acid residues at these sequence positions of mature hTlc (SEQ ID NO: 1). In some embodiments, an hTlc mutein of the disclosure comprises 15 or more of the above-mentioned mutated amino acid residues at these sequence positions of mature hTlc (SEQ ID NO: 1). In some embodiments, an hTlc mutein of the disclosure comprises 20 or more of the above-mentioned mutated amino acid residues at these sequence positions of mature hTlc (SEQ ID NO: 1).

In some embodiments, provided OX40-binding hTlc muteins may comprise, at one or more positions corresponding to positions 26-34, 55-56, 60, 101, 104-105, 108, 111, and 114 of the linear polypeptide sequence of mature hTlc (SEQ ID NO: 1), one or more of the following mutated amino acid residues: Arg 26→Trp; Glu 27→Asp; Phe 28→Cys; Pro 29→Asn; Glu 30→Gln; Met 31→Pro; Asn 32→Ile; Leu 33→Phe; Glu 34→Asp; Met 55→Ile; Leu 56→Phe; Arg 60→Lys; Cys 101→Ser; Glu 104→Gln; Leu 105→Cys; Lys 108→Ile; Arg 111→Pro; and Lys 114→Trp. In some embodiments, an hTlc mutein of the disclosure comprises two or more, such as 3, 4, 5, 6, 7, 8, 9, 10, 11, 12, 13, 14, 15, 16, 17, or 18 of the above-mentioned mutated amino acid residues at these sequence positions of mature hTlc (SEQ ID NO: 1). In some embodiments, an hTlc mutein of the disclosure comprises 10 or more of the above-mentioned mutated amino acid residues at these sequence positions of mature hTlc (SEQ ID NO: 1). In some embodiments, an hTlc mutein of the disclosure comprises 15 or more of the above-mentioned mutated amino acid residues at these sequence positions of mature hTlc (SEQ ID NO: 1).

In some embodiments, provided OX40-binding hTlc muteins may comprise, at one or more positions corresponding to positions 23, 26-34, 55-56, 58, 60-61, 101, 104-106, 108, 111, 114, and 153 of the linear polypeptide sequence of mature hTlc (SEQ ID NO: 1), one or more of the following mutated amino acid residues: Thr 23→Lys; Arg 26→Trp; Glu 27→Asp; Phe 28→Cys; Pro 29→Asn; Glu 30→Gln; Met 31→Pro; Asn 32→Ile; Leu 33→Phe; Glu 34→Asp; Met 55→Ile; Leu 56→Phe; Ser 58→Asp; Arg 60→Lys; Cys 61→Tyr; Cys 101→Ser; Glu 104→Gln; Leu 105→Cys; His 106→Pro; Lys 108→Ile; Arg 111→Pro; Lys 114→Trp; and Cys 153→Ser. In some embodiments, an hTlc mutein of the disclosure comprises two or more, such as 3, 4, 5, 6, 7, 8, 9, 10, 11, 12, 13, 14, 15, 16, 17, 18, 19, 20, 21, 22, or 23 of the above-mentioned mutated amino acid residues at these sequence positions of mature hTlc (SEQ ID NO: 1).

In some embodiments, provided OX40-binding hTlc muteins may comprise, at one or more positions corresponding to positions 5, 6, 8, 11, 19, 36, 37, 40, 52, 65, 79, 86, 113, 116, 121, 124, 137, 140, and 148 of the linear polypeptide sequence of mature hTlc (SEQ ID NO: 1), one or more of the following mutated amino acid residues: Ala 5→Thr; Ser 6→Thr; Glu 8→Lys; Gln 11→Arg; Leu 19→Met or Gln; Val 36→Asp; Thr 37→Ala; Thr 40→Ile; Lys 52→Glu; Lys 65→Ile; Ala 79→Thr; Ala 86→Thr; Val 113→Met or Leu; Val 116→Ala; Lys 121→Met; Leu 124→Lys; Arg 137→His; Ser 140→Arg; and Arg 148→Ser or Trp.

In some embodiments, provided OX40-binding hTlc muteins may comprise one of the following sets of mutated amino acid residues in comparison with the linear polypeptide sequence of mature hTlc (SEQ ID NO: 1):
(a) Arg 26→Trp; Glu 27→Asp; Phe 28→Cys; Pro 29→Asn; Glu 30→Gln; Met 31→Pro; Asn 32→Ile; Leu 33→Phe; Glu 34→Asp; Met 55→Ile; Leu 56→Phe; Ser 58→Asp; Arg 60→Lys; Cys 61→Tyr; Cys 101→Ser; Glu 104→Gln; Leu 105→Cys; His 106→Pro; Lys 108→Ile; Arg 111→Pro; Lys 114→Trp; and Cys 153→Ser;
(b) Leu 19→Gln; Thr 23→Lys; Arg 26→Trp; Glu 27→Asp; Phe 28→Cys; Pro 29→Asn; Glu 30→Gln; Met 31→Pro; Asn 32→Ile; Leu 33→Phe; Glu 34→Asp; Val 36→Asp; Thr 40→Ile; Met 55→Ile; Leu 56→Phe; Ser 58→Asp; Arg 60→Lys; Cys 61→Tyr; Ala 86→Thr; Cys 101→Ser; Glu 104→Gln; Leu 105→Cys; His 106→Pro; Lys 108→Ile; Arg 111→Pro; Val 113→Met; Lys 114→Trp; and Cys 153→Ser
(c) Leu 19→Met; Thr 23→Lys; Arg 26→Trp; Glu 27→Asp; Phe 28→Cys; Pro 29→Asn; Glu 30→Gln; Met 31→Pro; Asn 32→Ile; Leu 33→Phe; Glu 34→Asp; Met 55→Ile; Leu 56→Phe; Ser 58→Asp; Arg 60→Lys; Cys 61→Tyr; Cys 101→Ser; Glu 104→Gln; Leu 105→Cys; His 106→Pro; Lys 108→Ile; Arg 111→Pro; Val 113→Met; Lys 114→Trp; and Cys 153→Ser;
(d) Ala 5→Thr; Thr 23→Lys; Arg 26→Trp; Glu 27→Asp; Phe 28→Cys; Pro 29→Asn; Glu 30→Gln; Met 31→Pro; Asn 32→Ile; Leu 33→Phe; Glu 34→Asp; Lys 52→Glu; Met 55→Ile; Leu 56→Phe; Ser 58→Asp; Arg 60→Lys; Cys 61→Tyr; Cys 101→Ser; Glu 104→Gln; Leu 105→Cys; His 106→Pro; Lys 108→Ile; Arg 111→Pro; Val 113→Met; Lys 114→Trp; Arg 137→His; and Cys 153→Ser;
(e) Ser 6→Thr; Thr 23→Lys; Arg 26→Trp; Glu 27→Asp; Phe 28→Cys; Pro 29→Asn; Glu 30→Gln; Met 31→Pro; Asn 32→Ile; Leu 33→Phe; Glu 34→Asp; Thr 37→Ala; Met 55→Ile; Leu 56→Phe; Ser 58→Asp; Arg 60→Lys; Cys 61→Tyr; Lys 65→Ile; Ala 79→Thr; Cys 101→Ser; Glu 104→Gln; Leu 105→Cys; His 106→Pro; Lys 108→Ile; Arg 111→Pro; Lys 114→Trp; Val 116→Ala; and Cys 153→Ser;
(f) Ala 5→Thr; Thr 23→Lys; Arg 26→Trp; Glu 27→Asp; Phe 28→Cys; Pro 29→Asn; Glu 30→Gln; Met 31→Pro; Asn 32→Ile; Leu 33→Phe; Glu 34→Asp; Val 36→Asp; Met 55→Ile; Leu 56→Phe; Ser 58→Asp; Arg 60→Lys; Cys 61→Tyr; Cys 101→Ser; Glu 104→Gln; Leu 105→Cys; His 106→Pro; Lys 108→Ile; Arg 111→Pro; Lys 114→Trp; Arg 148→Ser; and Cys 153→Ser;
(g) Glu 8→Lys; Thr 23→Lys; Arg 26→Trp; Glu 27→Asp; Phe 28→Cys; Pro 29→Asn; Glu 30→Gln; Met 31→Pro; Asn 32→Ile; Leu 33→Phe; Glu 34→Asp; Val 36→Asp; Met 55→Ile; Leu 56→Phe; Ser 58→Asp; Arg 60→Lys; Cys 61→Tyr; Cys 101→Ser; Glu 104→Gln; Leu 105→Cys; His 106→Pro; Lys 108→Ile; Arg 111→Pro; Val 113→Leu; Lys 114→Trp; Lys 121→Met; and Cys 153→Ser;
(h) Gln 11→Arg; Thr 23→Lys; Arg 26→Trp; Glu 27→Asp; Phe 28→Cys; Pro 29→Asn; Glu 30→Gln; Met 31→Pro; Asn 32→Ile; Leu 33→Phe; Glu 34→Asp; Val 36→Asp; Met 55→Ile; Leu 56→Phe; Ser 58→Asp; Arg 60→Lys; Cys 61→Tyr; Cys 101→Ser; Glu 104→Gln; Leu 105→Cys; His 106→Pro; Lys 108→Ile; Arg 111→Pro; Lys 114→Trp; Ser 140→Arg; Arg 148→Trp; and Cys 153→Ser;
(i) Thr 23→Lys; Arg 26→Trp; Glu 27→Asp; Phe 28→Cys; Pro 29→Asn; Glu 30→Gln; Met 31→Pro; Asn 32→Ile; Leu 33→Phe; Glu 34→Asp; Val 36→Asp; Met 55→Ile; Leu 56→Phe; Ser 58→Asp; Arg 60→Lys; Cys 61→Tyr; Cys 101→Ser; Glu 104→Gln; Leu 105→Cys; His 106→Pro; Lys 108→Ile; Arg 111→Pro; Lys 114→Trp; and Cys 153→Ser;
(j) Thr 23→Lys; Arg 26→Trp; Glu 27→Asp; Phe 28→Cys; Pro 29→Asn; Glu 30→Gln; Met 31→Pro; Asn 32→Ile; Leu 33→Phe; Glu 34→Asp; Val 36→Asp; Met 55→Ile; Leu 56→Phe; Arg 60→Lys; Cys 61→Tyr; Cys 101→Ser; Glu 104→Gln; Leu 105→Cys; Lys 108→Ile; Arg 111→Pro; Lys 114→Trp; Leu 124→Lys; and Cys 153→Ser; or
(k) Thr 23→Lys; Arg 26→Trp; Glu 27→Asp; Phe 28→Cys; Pro 29→Asn; Glu 30→Gln; Met 31→Pro; Asn 32→Ile; Leu 33→Phe; Glu 34→Asp; Val 36→Asp; Met 55→Ile; Leu 56→Phe; Ser 58→Asp; Arg 60→Lys; Cys 101→Ser; Glu 104→Gln; Leu 105→Cys; His 106→Pro; Lys 108→Ile; Arg 111→Pro; and Lys 114→Trp.

In some embodiments, an OX40-binding hTlc mutein includes all but three, all but two, or all but one mutated amino acid residues of one of the aforementioned sets of mutated amino acid residues in comparison with the linear polypeptide sequence of mature hTlc (SEQ ID NO: 1).

In some embodiments, the residual region, i.e., the region differing from positions corresponding to positions 5, 6, 8, 11, 19, 23, 26-34, 36, 37, 40, 52, 55-56, 58, 60-61, 65, 79, 86, 101, 104-106, 108, 111, 113-114, 116, 121, 124, 137, 140, 148, and 153 of the linear polypeptide sequence of mature hTlc (SEQ ID NO: 1), of an OX40-binding hTlc mutein of the disclosure may comprise the wild-type (natural) amino acid sequence of the linear polypeptide sequence of mature hTlc outside the mutated amino acid sequence positions.

In some embodiments, an OX40-binding hTlc mutein of the disclosure has at least 70% sequence identity or at least 70% sequence homology to the sequence of mature hTlc (SEQ ID NO: 1). As an illustrative example, the mutein of the SEQ ID NO: 182 has an amino acid sequence identity or a sequence homology of approximately 84% with the amino acid sequence of the mature hTlc.

In some embodiments, an OX40-binding hTlc mutein of the disclosure comprises an amino acid sequence as set forth in any one of SEQ ID NOs: 174-184 or a fragment or variant thereof.

In some embodiments, an OX40-binding hTlc mutein of the disclosure has at least 75%, at least 80%, at least 85%, at least 90%, at least 95%, at least 98%, at least 99% or higher sequence identity to an amino acid sequence selected from the group consisting of SEQ ID NOs: 174-184.

The present disclosure also includes structural homologues of an OX40-binding hTlc mutein having an amino acid sequence selected from the group consisting of SEQ ID NOs: 173-183, which structural homologues have an amino acid sequence homology or sequence identity of more than about 60%, preferably more than 65%, more than 70%, more than 75%, more than 80%, more than 85%, more than 90%, more than 92% and most preferably more than 95% in relation to said hTlc mutein.

In some embodiments, provided OX40-binding hNGAL muteins may comprise a mutated amino acid residue at one or more (e.g., at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or even more) positions corresponding to positions 3, 21, 25-26, 28, 36, 40-41, 44, 49-50, 52, 55, 59, 60, 62-63, 65, 68, 70, 72-73, 75, 77-83, 87, 93, 96, 98, 100, 103, 106, 108, 114, 118, 125, 127, 129, 132, 134, 143, 150, 164, and 170 of the linear polypeptide sequence of mature hNGAL (SEQ ID NO: 2). In some embodiments, an hNGAL mutein of the disclosure comprises 10 or more mutated amino acid residues at one or more of the above-mentioned positions of the linear polypeptide sequence of mature hNGAL (SEQ ID NO: 2). In some embodiments, an hNGAL mutein of the disclosure comprises 15 or more mutated amino acid residues at one or more of the above-mentioned positions of the linear polypeptide sequence of mature hNGAL (SEQ ID NO: 2). In some embodiments, an hNGAL mutein of the disclosure comprises 20 or more mutated amino acid residues at one or more of the above-mentioned positions of the linear polypeptide sequence of mature hNGAL (SEQ ID NO: 2).

In some embodiments, provided OX40-binding hNGAL muteins may comprise, at one or more positions corresponding to positions 3, 21, 25-26, 28, 36, 40-41, 44, 49-50, 52, 55, 59, 60, 62-63, 65, 68, 70, 72-73, 75, 77-83, 87, 93, 96, 98, 100, 103, 106, 108, 114, 118, 125, 127, 129, 132, 134, 143, 150, 164, and 170 of the linear polypeptide sequence of mature hNGAL (SEQ ID NO: 2), one or more of the following mutated amino acid residues: Ser 3→Phe or Pro; Asn 21→Asp; Asn 25→Ser; Gln 26→Arg; Gln 28→His; Leu 36→Phe; Ala 40→Tyr; Ile 41→Trp or Arg; Glu 44→Gly; Gln 49→Gly; Lys 50→Glu or Thr; Tyr 52→Gln; Ile 55→Val; Lys 59→Arg; Glu 60→Lys; Tyr 62→Arg; Ser 63→Thr or Ala; Asn 65→Gln or Arg; Ser 68→Gly; Leu 70→Pro or Arg; Arg 72→Pro; Lys 73→His; Lys 75→Glu; Asp 77→His; Tyr 78→Asp or His; Trp 79→Asp; Ile 80→Thr; Arg 81→Val; Thr 82→Ile or Val; Phe 83→Leu; Cys 87→Ile, Ser, or Arg; Thr 93→Ile; Asn 96→Trp; Lys 98→Arg; Tyr 100→Asp; Leu 103→Ile; Tyr 106→Asp; Val 108→Ala; Asn 114→Asp; His 118→Tyr; Lys 125→Trp; Ser 127→Phe; Asn 129→Asp; Tyr 132→Trp; and Lys 134→Tyr; Glu 143→Ala; Glu 150→Gly; Gln 164→Asp; and Val 170→Ala. In some embodiments, an hNGAL mutein of the disclosure comprises two or more, such as 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or all mutated amino acid residues at these sequence positions of mature hNGAL (SEQ ID NO: 2). In some embodiments, an hNGAL mutein of the disclosure comprises 10 or more of the above-mentioned mutated amino acid residues at these sequence positions of mature hNGAL (SEQ ID NO: 2). In some embodiments, an hNGAL mutein of the disclosure comprises 15 or more of the above-mentioned mutated amino acid residues at these sequence positions of mature hNGAL (SEQ ID NO: 2). In some embodiments, an hNGAL mutein of the disclosure comprises 20 or more of the above-mentioned mutated amino acid residues at these sequence positions of mature hNGAL (SEQ ID NO: 2).

In some embodiments, provided OX40-binding hNGAL muteins may comprise, at one or more positions corresponding to positions 36, 40-41, 49, 52, 68, 72-73, 77, 79, 81, 87, 96, 100, 103, 106, 125, 127, 132, and 134 of the linear polypeptide sequence of mature hNGAL (SEQ ID NO: 2), one or more of the following mutated amino acid residues: Leu 36→Phe; Ala 40→Tyr; Ile 41→Trp or Arg; Gln 49→Gly; Tyr 52→Gln; Ser 68→Gly; Arg 72→Pro; Lys 73→His; Asp 77→His; Trp 79→Asp; Arg 81→Val; Cys 87→Ile, Ser, or Arg; Asn 96→Trp; Tyr 100→Asp; Leu 103→Ile; Tyr 106→Asp; Lys 125→Trp; Ser 127→Phe; Tyr 132→Trp; and Lys 134→Tyr. In some embodiments, an hNGAL mutein of the disclosure comprises two or more, such as 3, 4, 5, 6, 7, 8, 9, 10, 11, 12, 13, 14, 15, 16, 17, 18, 19, or 20 mutated amino acid residues at these sequence positions of mature hNGAL (SEQ ID NO: 2).

In some embodiments, provided OX40-binding hNGAL muteins may comprise, at one or more positions corresponding to positions 3, 21, 25-26, 28, 44, 50, 55, 59-60, 62-63, 65, 70, 75, 78, 80, 82-83, 93, 98, 108, 114, 118, 129, 143, 150, 164, and 170 of the linear polypeptide sequence of mature hNGAL (SEQ ID NO: 2), one or more of the following mutated amino acid residues: Ser 3→Phe or Pro; Asn 21→Asp; Asn 25→Ser; Gln 26→Arg; Gln 28→His; Glu 44→Gly; Lys 50→Glu or Thr; Ile 55→Val; Lys 59→Arg; Glu 60→Lys; Tyr 62→Arg; Ser 63→Thr or Ala; Asn 65→Gln or Arg; Leu 70→Pro or Arg; Lys 75→Glu; Tyr 78→Asp or His; Ile 80→Thr; Thr 82→Ile or Val; Phe 83→Leu; Thr 93→Ile; Lys 98→Arg; Val 108→Ala; Asn 114→Asp; His 118→Tyr; Asn 129→Asp; Glu 143→Ala; Glu 150→Gly; Gln 164→Asp; and Val 170→Ala.

some embodiments, provided OX40-binding hNGAL muteins may comprise one of the following sets of mutated amino acid residues in comparison with the linear polypeptide sequence of mature hNGAL (SEQ ID NO: 2):
(a) Gln 28→His; Leu 36→Phe; Ala 40→Tyr; Ile 41→Trp; Gln 49→Gly; Tyr 52→Gln; Ser 68→Gly; Arg 72→Pro; Lys 73→His; Asp 77→His; Trp 79→Asp; Arg 81→Val; Cys 87→Ser; Asn 96→Trp; Tyr 100→Asp; Leu 103→Ile; Tyr 106→Asp; Lys 125→Trp; Ser 127→Phe; Tyr 132→Trp; and Lys 134→Tyr;
(b) Leu 36→Phe; Ala 40→Tyr; Ile 41→Trp; Gln 49→Gly; Tyr 52→Gln; Glu 60→Lys; Ser 68→Gly; Arg 72→Pro; Lys 73→His; Lys 75→Glu; Asp 77→His; Trp 79→Asp; Arg 81→Val; Phe 83→Leu; Cys 87→Ile; Thr 93→Ile; Asn 96→Trp; Tyr 100→Asp; Leu 103→Ile; Tyr 106→Asp; Asn 114→Asp; Lys 125→Trp; Ser 127→Phe; Tyr 132→Trp; and Lys 134→Tyr;
(c) Leu 36→Phe; Ala 40→Tyr; Ile 41→Trp; Gln 49→Gly; Tyr 52→Gln; Ser 63→Thr; Ser 68→Gly; Leu 70→Pro; Arg 72→Pro; Lys 73→His; Asp 77→His; Trp 79→Asp; Arg 81→Val; Cys 87→Ser; Thr 93→Ile; Asn 96→Trp; Tyr 100→Asp; Leu 103→Ile; Tyr 106→Asp; Lys 125→Trp; Ser 127→Phe; Tyr 132→Trp; and Lys 134→Tyr;
(d) Leu 36→Phe; Ala 40→Tyr; Ile 41→Trp; Gln 49→Gly; Lys 50→Glu; Tyr 52→Gln; Ser 68→Gly; Arg 72→Pro; Lys 73→His; Asp 77→His; Trp 79→Asp; Ile 80→Thr; Arg 81→Val; Cys 87→Arg; Thr 93→Ile; Asn 96→Trp; Lys 98→Arg; Tyr 100→Asp; Leu 103→Ile; Tyr 106→Asp; Asn 114→Asp; Lys 125→Trp; Ser 127→Phe; Tyr 132→Trp; and Lys 134→Tyr;
(e) Ser 3→Phe; Leu 36→Phe; Ala 40→Tyr; Ile 41→Trp; Gln 49→Gly; Tyr 52→Gln; Ile 55→Val; Ser 68→Gly; Arg 72→Pro; Lys 73→His; Asp 77→His; Trp 79→Asp; Arg 81→Val; Phe 83→Leu; Cys 87→Ser; Thr 93→Ile; Asn 96→Trp; Lys 98→Arg; Tyr 100→Asp; Leu 103→Ile; Tyr 106→Asp; His 118→Tyr; Lys 125→Trp; Ser 127→Phe; Tyr 132→Trp; Lys 134→Tyr; and Glu 150→Gly;
(f) Gln 28→His; Leu 36→Phe; Ala 40→Tyr; Ile 41→Trp; Glu 44→Gly; Gln 49→Gly; Lys 50→Thr; Tyr 52→Gln; Tyr 62→Arg; Asn 65→Gln; Ser 68→Gly; Arg 72→Pro; Lys 73→His; Lys 75→Glu; Asp 77→His; Trp 79→Asp; Ile 80→Thr; Arg 81→Val; Thr 82→Ile; Phe 83→Leu; Cys 87→Ser; Asn 96→Trp; Tyr 100→Asp; Leu 103→Ile; Tyr 106→Asp; Asn 114→Asp; Lys 125→Trp; Ser 127→Phe; Tyr 132→Trp; and Lys 134→Tyr;
(g) Gln 28→His; Leu 36→Phe; Ala 40→Tyr; Ile 41→Trp; Gln 49→Gly; Lys 50→Glu; Tyr 52→Gln; Asn 65→Gln; Ser 68→Gly; Leu 70→Arg; Arg 72→Pro; Lys 73→His; Lys 75→Glu; Asp 77→His; Trp 79→Asp; Ile 80→Thr; Arg 81→Val; Thr 82 Ile; Cys 87→Ser; Asn 96→Trp; Tyr 100→Asp; Leu 103→Ile; Tyr 106→Asp; Asn 114→Asp; His 118→Tyr; Lys 125→Trp; Ser 127→Phe; Asn 129→Asp; Tyr 132→Trp; and Lys 134→Tyr;
(h) Asn 25→Ser; Leu 36→Phe; Ala 40→Tyr; Ile 41→Arg; Gln 49→Gly; Tyr 52→Gln; Lys 59→Arg; Ser 63→Ala; Ser 68→Gly; Leu 70→Pro; Arg 72→Pro; Lys 73→His; Asp 77→His; Trp 79→Asp; Arg 81→Val; Cys 87→Ser; Thr 93→Ile; Asn 96→Trp; Tyr 100→Asp; Leu 103→Ile; Tyr 106→Asp; Lys 125→Trp; Ser 127→Phe; Tyr 132→Trp; and Lys 134→Tyr;
(i) Asn 25→Ser; Leu 36→Phe; Ala 40→Tyr; Ile 41→Arg; Gln 49→Gly; Tyr 52→Gln; Lys 59→Arg; Ser 63→Ala; Asn 65→Gln; Ser 68→Gly; Leu 70→Pro; Arg 72→Pro; Lys 73→His; Asp 77→His; Tyr 78→Asp; Trp 79→Asp; Arg 81→Val; Thr 82→Ile; Cys 87→Ser; Thr 93→Ile; Asn 96→Trp; Tyr 100→Asp; Leu 103→Ile; Tyr 106→Asp; Asn 114→Asp; Lys 125→Trp; Ser 127→Phe; Tyr 132→Trp; Lys 134→Tyr; Glu 143→Ala; and Gln 164→Asp;
(j) Asn 25→Ser; Leu 36→Phe; Ala 40→Tyr; Ile 41→Arg; Gln 49→Gly; Tyr 52→Gln; Lys 59→Arg; Ser 63→Ala; Asn 65→Gln; Ser 68→Gly; Arg 72→Pro; Lys 73→His; Asp 77→His; Tyr 78→Asp; Trp 79→Asp; Arg 81→Val; Thr 82→Ile; Cys 87→Ser; Thr 93→Ile; Asn 96→Trp; Tyr 100→Asp; Leu 103→Ile; Tyr 106→Asp; Asn 114→Asp; Lys 125→Trp; Ser 127→Phe; Tyr 132→Trp; Lys 134→Tyr; Glu 143→Ala; and Gln 164→Asp;
(k) Ser 3→Pro; Asn 25→Ser; Leu 36→Phe; Ala 40→Tyr; Ile 41→Arg; Gln 49→Gly; Lys 50→Glu; Tyr 52→Gln; Lys 59→Arg; Ser 63→Ala; Asn 65→Gln; Ser 68→Gly; Leu 70→Pro; Arg 72→Pro; Lys 73→His; Asp 77→His; Trp 79→Asp; Ile 80→Thr; Arg 81→Val; Thr 82→Ile; Cys 87→Ser; Thr 93→Ile; Asn 96→Trp; Tyr 100→Asp; Leu 103→Ile; Tyr 106→Asp; Asn 114→Asp; Lys 125→Trp; Ser 127→Phe; Tyr 132→Trp; and Lys 134→Tyr;
(l) Asn 25→Ser; Gln 26→Arg; Leu 36→Phe; Ala 40→Tyr; Ile 41→Arg; Gln 49→Gly; Tyr 52→Gln; Lys 59→Arg; Ser 63→Ala; Asn 65→Gln; Ser 68→Gly; Leu 70→Pro; Arg 72→Pro; Lys 73→His; Asp 77→His; Tyr 78→His; Trp 79→Asp; Ile 80→Thr; Arg 81→Val; Thr 82→Ile; Cys 87→Ser; Thr 93→Ile; Asn 96→Trp; Tyr 100→Asp; Leu 103→Ile; Tyr 106→Asp; Lys 125→Trp; Ser 127→Phe; Tyr 132→Trp; Lys 134→Tyr; and Gln 164→Asp;
(m) Asn 21→Asp; Asn 25→Ser; Leu 36→Phe; Ala 40→Tyr; Ile 41→Arg; Gln 49→Gly; Tyr 52→Gln; Lys 59→Arg; Ser 63→Ala; Asn 65→Gln; Ser 68→Gly; Leu 70→Pro; Arg 72→Pro; Lys 73→His; Asp 77→His; Tyr 78→Asp; Trp 79→Asp; Ile 80→Thr; Arg 81→Val; Thr 82→Ile; Cys 87→Ser; Thr 93→Ile; Asn 96→Trp; Tyr 100→Asp; Leu 103→Ile; Tyr 106→Asp; Lys 125→Trp; Ser 127→Phe; Tyr 132→Trp; Lys 134→Tyr; and Gln 164→Asp;
(n) Asn 25→Ser; Leu 36→Phe; Ala 40→Tyr; Ile 41→Arg; Gln 49→Gly; Tyr 52→Gln; Lys 59→Arg; Ser 63→Ala; Asn 65→Gln; Ser 68→Gly; Leu 70→Pro; Arg 72→Pro; Lys 73→His; Lys 75→Glu; Asp 77→His; Trp 79→Asp; Ile 80→Thr; Arg 81→Val; Thr 82→Ile; Cys 87→Ser; Thr 93→Ile; Asn 96→Trp; Tyr 100→Asp; Leu 103→Ile; Tyr 106→Asp; Lys 125→Trp; Ser 127→Phe; Asn 129→Asp; Tyr 132→Trp; and Lys 134→Tyr;
(o) Asn 25→Ser; Leu 36→Phe; Ala 40→Tyr; Ile 41→Arg; Gln 49→Gly; Lys 50→Glu; Tyr 52→Gln; Lys 59→Arg; Ser 63→Ala; Asn 65→Arg; Ser 68→Gly; Leu 70→Pro; Arg 72→Pro; Lys 73→His; Lys 75→Glu; Asp 77→His; Trp 79→Asp; Arg 81→Val; Thr 82→Val; Cys 87→Ser; Thr 93→Ile; Asn 96→Trp; Tyr 100→Asp; Leu 103→Ile; Tyr 106→Asp; Val 108→Ala; Lys 125→Trp; Ser 127→Phe; Asn 129→Asp; Tyr 132→Trp; Lys 134→Tyr; Gln 164→Asp; and Val 170→Ala;
(p) Asn 25→Ser; Leu 36→Phe; Ala 40→Tyr; Ile 41→Arg; Gln 49→Gly; Tyr 52→Gln; Lys 59→Arg; Ser 63→Ala; Asn 65→Gln; Ser 68→Gly; Arg 72→Pro; Lys 73→His; Asp 77→His; Tyr 78→Asp; Trp 79→Asp; Arg 81→Val; Thr 82→Ile; Cys 87→Ser; Thr 93→Ile; Asn 96→Trp; Tyr 100→Asp; Leu 103→Ile; Tyr 106→Asp; Asn 114→Asp; Lys 125→Trp; Ser 127→Phe; Tyr 132→Trp; Lys 134→Tyr; and Gln 164→Asp;
(q) Asn 25→Ser; Leu 36→Phe; Ala 40→Tyr; Ile 41→Arg; Gln 49→Gly; Tyr 52→Gln; Lys 59→Arg; Ser 63→Ala; Asn 65→Gln; Ser 68→Gly; Arg 72→Pro; Lys 73→His; Asp 77→His; Tyr 78→Asp; Trp 79→Asp; Ile 80→Thr; Arg 81→Val; Thr 82 Ile; Cys 87→Ser; Thr 93→Ile; Asn 96→Trp; Tyr 100→Asp; Leu 103→Ile; Tyr 106→Asp; Asn 114→Asp; Lys 125→Trp; Ser 127→Phe; Tyr 132→Trp; Lys 134→Tyr; and Gln 164→Asp; or
(r) Asn 25→Ser; Leu 36→Phe; Ala 40→Tyr; Ile 41→Arg; Gln 49→Gly; Tyr 52→Gln; Lys 59→Arg; Ser 63→Ala; Asn 65→Gln; Ser 68→Gly; Leu 70→Pro; Arg 72→Pro; Lys 73→His; Asp 77→His; Tyr 78→Asp; Trp 79→Asp; Ile 80→Thr; Arg 81→Val; Thr 82→Ile; Cys 87→Ser; Thr 93→Ile; Asn 96→Trp; Tyr 100→Asp; Leu 103→Ile; Tyr 106→Asp; Asn 114→Asp; Lys 125→Trp; Ser 127→Phe; Tyr 132→Trp; Lys 134→Tyr; and Gln 164→Asp.

In some embodiments, an OX40-binding hNGAL mutein of the disclosure comprises the following set of mutated amino acid residues in comparison with the linear polypeptide sequence of mature hNGAL (SEQ ID NO: 2) Asn 25→Ser; Leu 36→Phe; Ala 40→Tyr; Ile 41→Arg; Gln 49→Gly; Tyr 52→Gln; Lys 59→Arg; Ser 63→Ala; Asn 65→Gln; Ser 68→Gly; Arg 72→Pro; Lys 73→His; Asp 77→His; Tyr 78→Asp; Trp 79→Asp; Arg 81→Val; Thr 82→Ile; Cys 87→Ser; Thr 93→Ile; Asn 96→Trp; Tyr 100→Asp; Leu 103→Ile; Tyr 106→Asp; Asn 114→Asp; Lys 125→Trp; Ser 127→Phe; Tyr 132→Trp; Lys 134→Tyr; Glu 143→Ala; and Gln 164→Asp and/or has at least 75%, at least 80%, at least 85%, at least 90%, at least 95%, at least 98%, at least 99%, or higher sequence identity to the amino acid sequence of SEQ ID NO: 194.

In some embodiments, an OX40-binding hNGAL mutein includes all but three, all but two, or all but one mutated amino acid residues of one of the aforementioned sets of mutated amino acid residues in comparison with the linear polypeptide sequence of mature hNGAL (SEQ ID NO: 2).

In some further embodiments, in the residual region, i.e., the region differing from positions 3, 21, 25-26, 28, 36, 40-41, 44, 49-50, 52, 55, 59, 60, 62-63, 65, 68, 70, 72-73, 75, 77-83, 87, 93, 96, 98, 100, 103, 106, 108, 114, 118, 125, 127, 129, 132, 134, 143, 150, 164, and 170 of the linear polypeptide sequence of mature hNGAL (SEQ ID NO: 2), an hNGAL mutein of the disclosure may include the wild-type (natural) amino acid sequence of mature hNGAL outside the mutated amino acid sequence positions.

In some embodiments, an OX40-binding hNGAL mutein of the disclosure has at least 70% sequence identity or at least 70% sequence homology to the sequence of mature hNGAL (SEQ ID NO: 2). As an illustrative example, the mutein of the SEQ ID NO: 194 has an amino acid sequence identity or a sequence homology of approximately 83% with the amino acid sequence of the mature hNGAL.

In some embodiments, an OX40-binding hNGAL mutein of the disclosure comprises an amino acid sequence as set forth in any one of SEQ ID NOs: 185-202 or a fragment or variant thereof.

In some embodiments, an OX40-binding hNGAL mutein of the disclosure has at least 75%, at least 80%, at least 85%, at least 90%, at least 95%, at least 98%, at least 99%, or higher sequence identity to an amino acid sequence selected from the group consisting of SEQ ID NOs: 185-202.

The present disclosure also includes structural homologues of an OX40-binding hNGAL mutein having an amino acid sequence selected from the group consisting of SEQ ID NOs: 185-202, which structural homologues have an amino acid sequence homology or sequence identity of at least 60%, preferably at least 65%, at least 70%, at least 75%, at least 80%, at least 85%, at least 90%, at least 92%, at least 95%, at least 98%, or at least 99% in relation to said hNGAL mutein.

In some embodiments, the OX40-targeting moiety for use in a multimeric protein of the disclosure may be or comprise an OX40-targeting scFv. Such scFv may, for example, be derived from a known OX40-targeting antibody, such as MED10562, BMS-986178 or PF-04518600.

### H. Exemplary Uses and Applications of the Multimeric Proteins.

In some embodiments, the present disclosure encompasses the use of a multimeric protein of the disclosure, a nucleic acid molecule of the disclosure, a composition comprising such multimeric protein and/or such nucleic acid molecule, and/or a cell, in particular an immune cell, such as a T cell, such as a CAR-T cell of the disclosure for use in therapy.

In some embodiments, the present disclosure encompasses a pharmaceutical composition comprising the multimeric protein of the disclosure, a nucleic acid molecule of the disclosure, and/or or a cell, in particular an immune cell, such as a T cell, such as a CAR-T cell of the disclosure.

In some embodiments, the present disclosure encompasses the use of a multimeric protein of the disclosure, a nucleic acid molecule of the disclosure, a composition comprising such multimeric protein and/or the nucleic acid molecule, and/or a cell, in particular an immune cell, such as a T cell, such as a CAR-T cell of the disclosure, for the manufacture of a medicament.

In some embodiments, the present disclosure encompasses a multimeric protein of the disclosure, a nucleic acid molecule of the disclosure, a composition comprising such multimeric protein and/or such nucleic acid molecule, a cell, in particular an immune cell, such as a T cell, such as a CAR-T cell of the disclosure, a pharmaceutical composition of the disclosure, and/or a medicament of the disclosure is for the treatment of cancer, e.g., GPC3- or PD-L1-positive cancer. In some embodiments, the cancer is a solid tumor.

In some embodiments, the present disclosure encompasses the use of a multimeric protein of the disclosure, or a composition comprising such multimeric protein, or a cell, in particular an immune cell, such as a T cell, such as a CAR-T cell of the disclosure, for co-stimulating T cells and/or activating downstream signaling pathways of 4-1BE (and/or, optionally, OX40). In some embodiments, the T cells are CD4+ T cells, are CD8+ T cells, or comprise both. The co-stimulated T cell and/or the T cell of which 4-1BB (and/or, optionally, OX40) downstream signaling pathways have been activated may be a T cell that expresses and/or secretes the multimeric protein and/or one of its monomer polypeptides. The co-stimulated T cell and/or the T cell of which 4-1BE (and/or, optionally, OX40) downstream signaling pathways have been activated may also be a bystander immune cell, such as a T cell, i.e. an immune cell or a T cell that does not express and/or secrete the multimeric protein and/or one of its monomer polypeptides. The bystander immune cell may however be in proximity to the cell that expresses and/or secretes the multimeric protein and/or one of its monomer polypeptides. The bystander immune cell may be another tumor infiltrating T cell. The present disclosure provides a method of co-stimulating T cells and/or activating downstream signaling pathways of 4-1EE (and/or, optionally, OX40), by administering a multimeric protein of the disclosure, or a composition comprising such multimeric protein, or a cell, in particular an immune cell, such as a T cell, such as a CAR-T cell of the disclosure.

In some embodiments, the present disclosure encompasses the use of a multimeric protein of the disclosure, or a composition comprising such multimeric protein, or a cell, in particular an immune cell, such as a T cell, such as a CAR-T cell of the disclosure, for inducing 4-1BB (and/or, optionally, OX40) clustering and/or activation on T cells. In some embodiments, the T cells are CD4+ T cells, are CD8+ T cells, or comprise both. The T cell, of which 4-1BB (and/or, optionally, OX40) clustering has been induced and/or which has been activated, may be a T cell that expresses and/or secretes the multimeric protein and/or one of its monomer polypeptides. The T cell, of which 4-1BB (and/or, optionally, OX40) clustering has been induced and/or which has been activated, may also be a bystander immune cell, such as a bystander T cell, i.e. an immune cell or a T cell that does not express and/or secrete the multimeric protein and/or one of its monomer polypeptides. The bystander immune cell may however be in proximity to the cell that expresses and/or secretes the multimeric protein and/or one of its monomer polypeptides. The bystander immune cell may be another tumor infiltrating T cell. The present disclosure provides a method of inducing 4-1BB (and/or, optionally, OX40) clustering and activation on T cells, by administering a multimeric protein of the disclosure, or a composition comprising such multimeric protein, or a cell, in particular an immune cell, such as a T cell, such as a CAR-T cell of the disclosure.

In some embodiments, the present disclosure encompasses the use of one or more multimeric proteins disclosed herein or of one or more compositions comprising such multimeric proteins for simultaneously binding of 4-1BB and GPC3 or 4-1BB and PD-L1. In some embodiments, the present disclosure encompasses the use of a multimeric protein of the disclosure, or a composition comprising such multimeric protein, or a cell, in particular an immune cell, such as a T cell, such as a CAR-T cell of the disclosure, for co-stimulating T cells and/or activating downstream signaling pathways of 4-1BB when engaging GPC3- or PD-L1-expressing tumor cells. In some embodiments, the T cells are CD4+ T cells, are CD8+ T cells, or comprise both. The co-stimulated T cell and/or the T cell of which 4-1BB downstream signaling pathways have been activated may a T cell that expresses and/or secretes the multimeric protein and/or one of its monomer polypeptides. The co-stimulated T cell and/or the T cell of which 4-1BB downstream signaling pathways have been activated may also be a bystander immune cell, such as a T cell, i.e. an immune cell or a T cell that does not express and/or secrete the multimeric protein and/or one of its monomer polypeptides. The bystander immune cell may however be in proximity to the cell that expresses and/or secretes the multimeric protein and/or one of its monomer polypeptides. The bystander immune cell may be another tumor infiltrating T cell. The present disclosure provides a method of inducing 4-1BB clustering and activation on T cells when engaging GPC3- or PD-L1-expressing tumor cells, by administering a multimeric protein of the disclosure, or a composition comprising such multimeric protein, or a cell, in particular an immune cell, such as a T cell, such as a CAR-T cell of the disclosure.

In some embodiments, the present disclosure encompasses the use of one or more multimeric proteins disclosed herein or of one or more compositions comprising such multimeric proteins, or a cell, in particular an immune cell, such as a T cell, such as a CAR-T cell of the disclosure, for simultaneously binding of 4-1BB and a tumor associated antigen. In some embodiments, the present disclosure encompasses the use of a multimeric protein of the disclosure, or a composition comprising such multimeric protein, or a cell, in particular an immune cell, such as a T cell, such as a CAR-T cell of the disclosure, for co-stimulating T cells and/or activating downstream signaling pathways of 4-1BE when engaging TAA-expressing tumor cells or tumors. The co-stimulated T cell and/or the T cell of which 4-1BB downstream signaling pathways have been activated may a T cell that expresses and/or secretes the multimeric protein and/or one of its monomer polypeptides. The co-stimulated T cell and/or the T cell of which 4-1BB downstream signaling pathways have been activated may also be a bystander immune cell, such as a T cell, i.e. an immune cell or a T cell that does not express and/or secrete the multimeric protein and/or one of its monomer polypeptides. The bystander immune cell may however be in proximity to the cell that expresses and/or secretes the multimeric protein and/or one of its monomer polypeptides. The bystander immune cell may be another tumor infiltrating T cell. The present disclosure provides a method of inducing 4-1BB clustering and activation on T cells when engaging GPC3- or PD-L1-expressing tumor cells, by administering a multimeric protein of the disclosure, or a composition comprising such multimeric protein, or a cell, in particular an immune cell, such as a T cell, such as a CAR-T cell of the disclosure.

In some embodiments, the present disclosure provides multimeric proteins that simultaneously bind 4-1BB and GPC3 or PD-L1, or a cell that expresses and/or secretes the multimeric protein and/or one of its monomer polypeptides, for use such as anti-tumor and/or anti-infection agents, and immune modulators. In some embodiments, multimeric proteins of the disclosure may simultaneously target GPC3-expressing tumor cells (such as HCC, melanoma, Merkel cell carcinoma, Wilm's tumor, and hepatoblastoma cells) or PD-L1-expressing tumor cells, and activate lymphocytes of the host immune system adjacent to such tumor cells.

Additional objects, advantages, and features of this disclosure will become apparent to those skilled in the art upon examination of the following Examples and the attached Figures thereof, which are not intended to be limiting. Thus, it should be understood that although the present disclosure is specifically disclosed by exemplary embodiments and optional features, modification and variation of the disclosures embodied therein herein disclosed may be resorted to by those skilled in the art and that such modifications and variations are considered to be within the scope of this disclosure.

### I. Production of the Multimeric Proteins.

In some embodiments, the present disclosure provides nucleic acid molecules (DNA and RNA) that include nucleotide sequences encoding provided multimeric proteins. In some embodiments, the present disclosure provides nucleic acid molecules (DNA and RNA) that include nucleotide sequences encoding provided monomer polypeptides comprised in the multimeric proteins. In some embodiments, the disclosure encompasses a cell containing a provided nucleic acid molecule. Since the degeneracy of the genetic code permits substitutions of certain codons by other codons specifying the same amino acid, the disclosure is not limited to a specific nucleic acid molecule encoding a multimeric protein or monomer polypeptide comprised in the multimeric protein as described herein, rather, encompassing all nucleic acid molecules that include nucleotide sequences encoding a functional multimeric protein or monomer polypeptide comprised in the multimeric protein. In this regard, the present disclosure also relates to nucleotide sequences encoding provided multimeric proteins or monomer polypeptides comprised in the multimeric proteins. Exemplary nucleotide sequences provided by the present disclosure encoding the monomer polypeptides of SEQ ID NOs: 38-55 and 164-167 are shown in SEQ ID NOs: 144-161 and 168-171, respectively. Also provided herein are variants of these nucleotide sequences having at least 85%, at least 90%, at least 95%, at least 96%, at least 97%, at least 98% or at least 99% sequence identity a nucleotide sequence selected from the group consisting of SEQ ID NOs: 144-161 and 168-171 and encoding a monomer polypeptide of the present disclosure.

A nucleic acid molecule, such as DNA, is referred to as “capable of expressing a nucleic acid molecule” or “able to allow expression of a nucleotide sequence” if it includes sequence elements that contain information regarding to transcriptional and/or translational regulation, and such sequences are “operably linked” to the nucleotide sequence encoding the protein. An operable linkage is a linkage in which the regulatory sequence elements and the sequence to be expressed are connected in a way that enables gene expression. The precise nature of the regulatory regions necessary for gene expression may vary among species, but in general these regions include a promoter, which, in prokaryotes, contains both the promoter per se, i.e., DNA elements directing the initiation of transcription, as well as DNA elements which, when transcribed into RNA, will signal the initiation of translation. Such promoter regions normally include 5′ non-coding sequences involved in initiation of transcription and translation, such as the −35/−10 boxes and the Shine-Dalgarno element in prokaryotes or the TATA box, CAAT sequences, and 5′-capping elements in eukaryotes. These regions can also include enhancer or repressor elements as well as translated signal and leader sequences for targeting the native protein to a specific compartment of a host cell.

In addition, 3′ non-coding sequences may contain regulatory elements involved in transcriptional termination, polyadenylation or the like. If, however, these termination sequences are not satisfactorily functional in a particular host cell, then they may be substituted with signals functional in that cell.

Therefore, a nucleic acid molecule of the disclosure may be “operably linked” to one or more regulatory sequences, such as a promoter sequence, to allow expression of this nucleic acid molecule. In some embodiments, a nucleic acid molecule of the disclosure includes a promoter sequence and a transcriptional termination sequence. Suitable prokaryotic promoters are, for example, the tet promoter, the lacUV5 promoter or the T7 promoter. Examples of promoters useful for expression in eukaryotic cells are the SV40 promoter or the CMV promoter.

In some embodiments, a nucleic acid molecule encoding a moiety or domain of a provided monomer polypeptide comprised in the multimeric protein disclosed in this application may be “operably linked” to another nucleic acid molecule encoding a moiety or domain of the disclosure to allow expression of a multimeric protein disclosed herein.

In some embodiments, provided nucleic acid molecules can also be part of a vector or any other kind of cloning vehicle, such as a plasmid, a phagemid, a phage, a baculovirus, a cosmid or an artificial chromosome. In some embodiments, a provided nucleic acid molecule can also be comprised in the genomic DNA of a host cell. In some embodiments, a provided nucleic acid molecule can be comprised in an expression vector. Such expression vector may be a viral vector. Viral vectors for expression in animal cells, such as mammalian cells are known in the art. Viral vectors for expression in immune cells are e.g. disclosed in WO 2016/113203 A1, Chmielewski et al. 2011 Cancer Res 71(17): 5697-706; Zhang et al. 2011 Mol Ther 19(4): 751-9; Pegram et al. 2012 Blood 119(18): 4133-41 and Pegram et al. 2014 Leukemia 29(2):415-22, which are incorporated herewith by reference. In some embodiments, the nucleic acid molecule can be comprised in a nanoparticle. In some embodiments, the nucleic acid molecule can be comprised in a liposome. For example, mRNA encoding a multimeric protein of the disclosure or a monomer polypeptide thereof can be comprised in a nanoparticle or a liposome.

In some embodiments, a provided nucleic acid molecule may be included in a phagemid. As used in this context, a phagemid vector denotes a vector encoding the intergenic region of a temperate phage, such as M13 or f1, or a functional part thereof fused to the cDNA of interest. For example, in some embodiments, after superinfection of bacterial host cells with such a provided phagemid vector and an appropriate helper phage (e.g., M13K07, VCS-M13 or R408) intact phage particles are produced, thereby enabling physical coupling of the encoded heterologous cDNA to its corresponding polypeptide displayed on the phage surface (Lowman, 1997, Rodi and Makowski, 1999).

In accordance with various embodiments, cloning vehicles can include, aside from the regulatory sequences described above and a nucleic acid sequence encoding a multimeric protein as described herein, replication and control sequences derived from a species compatible with the host cell that is used for expression as well as selection markers conferring a selectable phenotype on transformed or transfected cells. Large numbers of suitable cloning vectors are known in the art and are commercially available.

The disclosure also relates, in some embodiments, to methods for the production of multimeric proteins of the disclosure starting from a nucleic acid coding for a multimeric protein or any monomer polypeptides therein. In some embodiments, a provided method can be carried out in vivo, wherein a provided multimeric protein can, for example, be produced in a bacterial or eukaryotic host organism. The multimeric protein may further be isolated from the host organism or its culture. It is also possible to produce a multimeric protein of the disclosure in vitro, for example, using an in vitro translation system.

When producing a multimeric protein in vivo, a nucleic acid encoding a multimeric protein may be introduced into a suitable bacterial or eukaryotic host organism using recombinant DNA technology well known in the art. In some embodiments, a DNA molecule encoding a multimeric protein as described herein, and in particular a cloning vector containing the coding sequence of such a multimeric protein can be transformed into a host cell capable of expressing the gene. Transformation can be performed using standard techniques. Thus, the disclosure is also directed to host cells containing a nucleic acid molecule as disclosed herein.

In some embodiments, transformed host cells may be cultured under conditions suitable for expression of the nucleotide sequence encoding a multimeric protein of the disclosure. In some embodiments, host cells can be prokaryotic, such as Escherichia coli (E. coli) or Bacillus subtilis, or eukaryotic, such as Saccharomyces cerevisiae, Pichia pastoris, SF9 or High5 insect cells, immortalized mammalian cell lines (e.g., HeLa cells or CHO cells) or primary mammalian cells.

In some embodiments, where a lipocalin mutein of the disclosure, including as comprised in a multimeric protein disclosed herein, includes intramolecular disulfide bonds, it may be preferred to direct the nascent protein to a cell compartment having an oxidizing redox milieu using an appropriate signal sequence. Such an oxidizing environment may be provided by the periplasm of Gram-negative bacteria such as E. coli, in the extracellular milieu of Gram-positive bacteria or the lumen of the endoplasmic reticulum of eukaryotic cells and usually favors the formation of structural disulfide bonds.

In some embodiments, it is also possible to produce a multimeric protein of the disclosure in the cytosol of a host cell, preferably E. coli. In this case, a provided multimeric protein can either be directly obtained in a soluble and folded state or recovered in the form of inclusion bodies, followed by renaturation in vitro. A further option is the use of specific host strains having an oxidizing intracellular milieu, which may thus allow the formation of disulfide bonds in the cytosol (Venturi et al., 2002).

In some embodiments, a multimeric protein of the disclosure as described herein may be not necessarily generated or produced, in whole or in part, via use of genetic engineering. Rather, such protein can also be obtained by any of the many conventional and well-known techniques such as plain organic synthesis strategies, solid phase-assisted synthesis techniques, commercially available automated synthesizers, or by in vitro transcription and translation. It is, for example, possible that promising multimeric proteins or lipocalin muteins included in such multimeric proteins are identified using molecular modeling, synthesized in vitro, and investigated for the binding activity for the target(s) of interest. Methods for the solid phase and/or solution phase synthesis of proteins are well known in the art (see e.g. Bruckdorfer et al., 2004).

In some embodiments, a multimeric protein of the disclosure may be produced by in vitro transcription/translation employing well-established methods known to those skilled in the art.

In some further embodiments, multimeric proteins as described herein may also be prepared by conventional recombinant techniques alone or in combination with conventional synthetic techniques.

Moreover, in some embodiments, a multimeric protein according to the present disclosure may be obtained by conjugating together individual subunits, e.g., single chain variable fragments and lipocalin muteins as included in the multimeric protein. Such conjugation can be, for example, achieved through all forms of covalent or non-covalent linkage using conventional methods.

One preferred application of the present invention is in armored cell therapy. Recombinant T cells, such as CAR-T cells can be engineered with the capacity to secrete proinflammatory cytokines. In this vein, the engineered CAR-T can lead to accumulation of a proinflammatory cytokine in the tumor microenvironment where the CAR-T traffics. A pro-inflammatory cytokine may be desired to facilitate recruiting a second wave of immune cells in a locally restricted fashion to initiate a more complete and potentially target-independent attack of the cells of the tumor. This approach has been described in particular utilizing an engineered single-chain variant of Interleukin 12, thereafter called sclL-12.

The ability to engineer a T cell to secrete yet other types of therapeutic proteins would be desirable, further expanding the repertoire to therapeutic modalities available in the tumor microenvironment. T cells secreting the multimeric protein of the invention meet this need.

Such construct can be based on T cells with a defined specificity (either without genetic manipulation or by transduction with a CAR or a recombinant TCR) that are equipped with the capacity to secrete multimeric proteins of the invention or the respective monomer polypeptides, which may then self-assemble into a multimeric protein. The skilled worker will appreciate that this approach can also be based on other cell types, such as NK cells or B cells

In some embodiments, the multimeric protein according to the present disclosure may be expressed and secreted by a cell. In some embodiments, the cell expresses and secretes the multimeric protein. In some embodiments, the cell expresses and secretes one or more monomer polypeptides. The monomer polypeptides may then self-assemble to a multimeric protein. The expression and secretion of the monomer polypeptide and/or the multimeric protein can either occur in vitro or in vivo.

For in vivo applications, it is advantageous when expression and/or secretion of the monomer polypeptide and/or the multimeric protein is at the desired tissue or site. For example, it might be desired that expression and/or secretion is in a tumor, in a tumor stroma, in a tumor microenvironment, or in proximity to a tumor.

In some embodiments, the cell is an immune cell. An “immune cell” as used herein refers to a cell that is part of the immune system and helps the body fight infections and other diseases. Immune cells include neutrophils, eosinophils, basophils, mast cells, monocytes, macrophages, dendritic cells, natural killer (NK) cells, and lymphocytes, such as B cells and/or T cells. The immune cell may be recombinant. Preferred immune cells are T cells. In some embodiments, the T cell may be a CD8+ T cell. In some embodiments, the T cell may be a CD4+ T cell. In some embodiments, the T cell may be a CAR-T cell. In some embodiments, the immune cell, in particular the T cell, may comprise a recombinant antigen receptor. Such recombinant antigen receptor may be a chimeric antigen receptor (CAR). Such recombinant antigen receptor may be a T cell receptor. The immune cell, in particular T cell, may express 4-1BB. It is understood that the cell, in particular the immune cell, may be a human cell, e.g. a human T cell.

The skilled worker will appreciate methods useful to prepare multimeric proteins contemplated by the present disclosure but whose protein or nucleic acid sequences are not explicitly disclosed herein. As an overview, such modifications of the amino acid sequence include, e.g., directed mutagenesis of single amino acid positions to simplify sub-cloning of a protein gene or its parts by incorporating cleavage sites for certain restriction enzymes. Also, these mutations can be incorporated to further improve the affinity of a multimeric protein for its targets (e.g., 4-1EE, OX40, PD-L1 and GPC3). Furthermore, mutations can be introduced to modulate one or more characteristics of the protein such as to improve folding stability, serum stability, protein resistance or water solubility or to reduce aggregation tendency, if necessary.

The invention may further be characterized by following items.

Item 1. A multimeric protein comprising at least three monomer polypeptides, wherein each monomer polypeptide comprises (1) a first 4-1BB-targeting moiety (T1), and (2) an oligomerization moiety (O).

Item 2. The multimeric protein of item 1, wherein the first 4-1BB-targeting moiety (T1) is fused at its N-terminus or C-terminus to the C-terminus or N-terminus, respectively, of the oligomerization moiety (O) via a linker (L).

Item 3. The multimeric protein of item 1 or 2, wherein the monomer polypeptide comprises at least one additional targeting moiety (T2).

Item 4. The multimeric protein of any one of items 1-3, wherein the monomer polypeptide comprises an additional targeting moiety (T2), wherein the additional targeting moiety is placed in tandem with the first 4-1BB-targeting moiety (T1).

Item 5. The multimeric protein of item 4, wherein the monomer polypeptide has one of the following configurations:
(a) T1-L′-T2-L-O;
(b) T2-L′-T1-L-O;
(c) O-L-T1-L′-T2; or
(d) O-L-T2-L′-T1 wherein L′ is a linker that is the same as or different from L.

Item 6. The multimeric protein of any one of items 1-3, wherein the monomer polypeptide comprises an additional targeting moiety (T2), wherein the additional targeting moiety (T2) is linked to a different terminus of the oligomerization moiety (O) than the first 4-1BB-targeting moiety (T1).

Item 7. The multimeric protein of item 6, wherein the monomer polypeptide has one of the following configurations:
(a) T1-L-O-L′-T2; or
(b) T2-L′-O-L-T1
   wherein L′ is a linker that is the same as or different from L.

Item 8. The multimeric protein of any one of items 3-7, wherein the additional targeting moiety (T2) is a second 4-1BB-targeting moiety.

Item 9. The multimeric protein of item 8, wherein the second 4-1BB-targeting moiety is the same as the first 4-1BB-targeting moiety (T1).

Item 10. The multimeric protein of any one of items 3-7, wherein the additional targeting moiety (T2) is a moiety that targets a tumor associated antigen.

Item 11. The multimeric protein of any one of items 3-7, wherein the additional targeting moiety (T2) is a moiety that targets a tumor associated antigen and is a lipocalin mutein.

Item 12. The multimeric protein of any one of items 3-7, wherein the additional targeting moiety (T2) is a moiety that targets a tumor associated antigen and is an antibody or an antigen-binding domain or derivative thereof.

Item 13. The multimeric protein of any one of items 3-7, wherein the additional targeting moiety (T2) is a moiety that targets a tumor associated antigen and is a single chain variable fragment (scFv).

Item 14. The multimeric protein of any one of items 3-7, wherein the additional targeting moiety (T2) is a GPC3-targeting moiety.

Item 15. The multimeric protein of any one of items 3-7, wherein the additional targeting moiety (T2) is a GPC3-targeting moiety that is a lipocalin mutein.

Item 16. The multimeric protein of item 15, wherein the lipocalin mutein has at least 85% sequence identity to an amino acid sequence selected from the group consisting of SEQ ID NOs: 74-97.

Item 17. The multimeric protein of any one of items 3-7, wherein the additional targeting moiety (T2) is a GPC3-targeting moiety that is an antibody or an antigen-binding domain or derivative thereof.

Item 18. The multimeric protein of any one of items 3-7, wherein the additional targeting moiety (T2) is a GPC3-targeting moiety that is a single chain variable fragment (scFv).

Item 19. The multimeric protein of any one of items 3-7, wherein the additional targeting moiety (T2) is a PD-L1-targeting moiety.

Item 20. The multimeric protein of any one of items 3-7, wherein the additional targeting moiety (T2) is a PD-L1-targeting moiety that is a single chain variable fragment (scFv).

Item 21. The multimeric protein of item 21, wherein the scFv has at least 85% sequence identity to the amino acid sequence shown in SEQ ID NO: 172.

Item 22. The multimeric protein of any one of items 3-7, wherein the additional targeting moiety (T2) is a T cell activation enhancing targeting moiety.

Item 23. The multimeric protein of any one of items 3-7, wherein the additional targeting moiety (T2) is a T cell activation enhancing targeting moiety that is a lipocalin mutein.

Item 24. The multimeric protein of any one of items 3-7, wherein the additional targeting moiety (T2) is a T cell activation enhancing targeting moiety that is an antibody or an antigen-binding domain or derivative thereof.

Item 25. The multimeric protein of any one of items 3-7, wherein the additional targeting moiety (T2) is a T cell activation enhancing targeting moiety that is a single chain variable fragment (scFv).

Item 26. The multimeric protein of any one of items 3-7, wherein the additional targeting moiety (T2) is an OX40-targeting moiety.

Item 27. The multimeric protein of any one of items 3-7, wherein the additional targeting moiety (T2) is an OX40-targeting moiety that is a lipocalin mutein.

Item 28. The multimeric protein of item 27, wherein the lipocalin mutein has at least 85% sequence identity to an amino acid sequence selected from the group consisting of SEQ ID NOs: 174-202.

Item 29. The multimeric protein of any one of items 1-28, wherein the first 4-1BB-targeting moiety (T1) is a lipocalin mutein.

Item 30. The multimeric protein of any one of items 1-29, wherein the first 4-1BB-targeting moiety (T1) is a lipocalin mutein having at least 85% sequence identity to an amino acid sequence selected from the group consisting of SEQ ID NOs: 56-71.

Item 31. The multimeric protein of any one of items 1-30, wherein the oligomerization moiety (O) is capable of promoting trimerization.

Item 32. The multimeric protein of any one of items 1-31, wherein the oligomerization moiety (O) is a trimerization domain of a collagen.

Item 33. The multimeric protein of any one of items 1-32, wherein the oligomerization moiety (O) has at least 85% sequence identity to an amino acid sequence selected from the group consisting of SEQ ID NOs: 35-37.

Item 34. The multimeric protein of any one of items 1-33, wherein the multimeric protein is a trimeric protein.

Item 35. The multimeric protein of any one of items 1-30 and 33, wherein the multimeric protein is a tetrameric protein.

Item 36. The multimeric protein of any one of items 2-35, wherein the linker (L) has at least 85% sequence identity to an amino acid sequence selected from the group consisting of SEQ ID NOs: 12-28.

Item 37. The multimeric protein of any one of items 1-36, wherein the multimeric protein has at least 85% sequence identity to an amino acid sequence selected from the group consisting of SEQ ID NOs: 38-55 and 164-167.

Item 38. The multimeric protein of any one of items 1-37, wherein the multimeric protein comprises an amino acid sequence selected from the group consisting of SEQ ID NOs: 38-55 and 164-167.

Item 39. The multimeric protein of any one of items 1-38, wherein the multimeric protein is capable of binding 4-1BB with an apparent K_{D} value of about 0.68 nM or lower.

Item 40. The multimeric protein of any one of items 1-39, wherein the multimeric protein is capable of binding 4-1BB with an apparent K_{D} value lower than the K_{D} value of the 4-1BB-targeting lipocalin mutein that is included in the monomer polypeptide.

Item 41. The multimeric protein of item 39 or 40, wherein the apparent K_{D} value is determined by surface plasmon resonance (SPR).

Item 42. The multimeric protein of any one of items 1-41, wherein the multimeric protein is cross-reactive with cynomolgus 4-11BE.

Item 43. A nucleic acid molecule comprising a nucleotide sequence encoding a monomer polypeptide comprised in a multimeric protein of any one of items 1-42.

Item 44. The nucleic acid molecule of item 43, wherein the nucleic acid molecule is operably linked to a regulatory sequence to allow expression of said nucleic acid molecule.

Item 45. The nucleic acid molecule of item 43 or 44, wherein the nucleic acid molecule is comprised in a vector or in a phagemid vector.

Item 46. The nucleic acid molecule of any one of items 43 or 44, wherein the nucleic acid molecule is comprised in a viral vector, in a nanoparticle, or a liposome.

Item 47. The nucleic acid molecule of any one of items 43-46, wherein the nucleic acid molecule is comprised in the genomic DNA of a host cell.

Item 48. A cell containing a nucleic acid molecule of any one of items 43-47 and/or expressing the multimeric protein of any one of items 1-42 and/or expressing a monomer polypeptide as defined in any one of items 1-42.

Item 49. The cell of item 48, wherein the cell secretes the multimeric protein and/or the monomer polypeptide.

Item 50. The cell of item 48, wherein the cell secretes the monomer polypeptide.

Item 51. The cell of item 50, wherein the monomer polypeptide self-assembles to a multimeric protein after secretion.

Item 52. The cell of any one of items 48-51, wherein the cell is an immune cell.

Item 53. The cell of item 52, wherein the cell is a T cell.

Item 54. The cell of item 53, wherein the cell is a CD8+ T cell.

Item 55. The cell of item 53, wherein the cell is a CD4+ T cell.

Item 56. The cell of any one of items 52-55, wherein the cell comprises a recombinant antigen receptor.

Item 57. The cell of item 56, wherein the recombinant antigen receptor is a chimeric antigen receptor (CAR).

Item 58. The cell of item 56, wherein the recombinant antigen receptor is a T cell receptor (TCR).

Item 59. The cell of any one of items 52-57, wherein the cell is a CAR-T cell.

Item 60. The cell of any one of items 52-59, wherein the cell expresses 4-1EE.

Item 61. The cell of any one of items 52-60, wherein the cell is a human cell.

Item 62. A method of producing the multimeric protein of any one of items 1-42, wherein the multimeric protein is produced starting from the nucleic acid coding for the monomer polypeptides comprised in the multimeric protein.

Item 63. The method of item 62, wherein the multimeric protein is produced in a bacterial or eukaryotic host organism.

Item 64. A use of the multimeric protein of any one of items 1-42 or a composition comprising such multimeric protein or a cell of any one of items 48-61 for inducing 4-1BB (and/or, optionally, OX40) clustering and activation on T cells.

Item 65. A use of the multimeric protein of any one of items 1-42 or a composition comprising such multimeric protein or a cell of any one of items 48-61 for co-stimulating T cells and/or activating downstream signaling pathways of 4-1EE (and/or, optionally, OX40).

Item 66. A use of the multimeric protein of any one of items 1-42 or a composition comprising such multimeric protein or a cell of any one of items 48-61 for co-stimulating T cells when engaging GPC3- or PD-L1-expressing tumor cells.

Item 67. The use of any one of items 64-66, wherein the T cell is a T cell expressing the multimeric protein and/or one of its monomer polypeptides.

Item 68. The use of any one of items 64-66, wherein the T cell is a T cell not expressing the multimeric protein and/or one of its monomer polypeptides.

Item 69. A pharmaceutical composition comprising one or more multimeric proteins of any one of items 1-42 and/or one or more cells of any one of items 48-61.

Item 70. The multimeric protein of any one of items 1-42 and/or the cell of any one of items 48-61 for use in a therapy.

Item 71. The multimeric protein and/or the cell for use of item 70, wherein the use is in the treatment of cancer.

Item 72. Use of a multimeric protein of any one of items 1-42 and/or the cell of any one of items 48-61 for the manufacture of a medicament.

Item 73. The use of item 72, wherein the medicament is for the treatment of cancer.

### V. EXAMPLES

### Example 1: Expression and Analysis of Representative Multimeric Proteins

In this Example, multimeric proteins were generated by the self-assembly of constituting monomer polypeptides. The monomer polypeptides were generated by fusing together a 4-11BB-targeting moiety, an oligomerization moiety, and optionally one or more additional targeting moieties.

Representative monomer polypeptides were generated by fusing one or more 4-1BB-targeting lipocalin muteins of the disclosure such as SEQ ID NO: 64 to the N-terminus, C-terminus, or both N- and C-termini of the human collagen XVIII trimerization domain (SEQ ID NO: 35) via a linker such as a linker shown in any one of SEQ ID NOs: 12-28. The different formats that were generated are depicted in FIGS. 1A and 1B. In addition, exemplary bispecific monomer polypeptides were generated by fusing a 4-1BB-targeting lipocalin mutein of the disclosure such as SEQ ID NO: 64 and (1) a GPC3-targeting moiety of the disclosure such as SEQ ID NO: 90 or SEQ ID NO: 98, (2) an OX40-targeting moiety of the disclosure such as SEQ ID NO: 194, or (3) an PD-L1-targeting moiety of the disclosure such as SEQ ID NO: 172 to the N-terminus, C-terminus, or both N- and C-termini of the human collagen XVIII trimerization domain (SEQ ID NO: 35) via linkers such as a linker shown in any one of SEQ ID NOs: 12-28. The different formats that were generated are depicted in FIG. 1C.

Additional bispecific formats can be generated by replacing one of the 4-1BB targeting moieties of the monomer polypeptides shown in FIG. 1B with a moiety targeting another target (i.e., other than 4-1BB). Exemplary bispecific monomer polypeptides were generated by fusing, via linkers, (1) the C-terminus of a 4-1BB-targeting lipocalin mutein of the disclosure such as SEQ ID NO: 64 to the N-terminus of an OX40-targeting lipocalin mutein of the disclosure such as SEQ ID NO: 194 and the C-terminus of the OX40-targeting lipocalin mutein to the N-terminus of the human collagen XVIII trimerization domain (SEQ ID NO: 35), resulting, e.g., in the monomer polypeptide of SEQ ID NO: 165, or (2) the C-terminus of an OX40-targeting lipocalin mutein of the disclosure such as SEQ ID NO: 194 to the N-terminus of a 4-1BB-targeting lipocalin mutein of the disclosure such as SEQ ID NO: 64 and the C-terminus of the 4-1BB-targeting lipocalin mutein to the N-terminus of the human collagen XVIII trimerization domain (SEQ ID NO: 35), resulting, e.g., in the monomer polypeptide of SEQ ID NO: 166.

The constructs of the monomer polypeptides were C-terminally fused to a myc-His-tag (SEQ ID NO: 131) and were generated by gene synthesis and cloned into a mammalian expression vector. They were then transiently expressed in Expi293F or ExpiCHO-S cells (Life Technologies) and allowed to self-assemble. The yields of exemplary multimeric proteins after His-tag purification followed by size-exclusion chromatography in phosphate-buffered saline (PBS) are summarized in Table 1. After SEC purification, the fractions containing multimeric proteins at the desired oligomerization state were pooled and analyzed again using analytical SEC (see Table 1).

TABLE 1
Transient expression

| SEQ ID | [mg] after | Main species |
|---|---|---|
| NO | purification | aSEC [%] |
| 38 | 109.4 | 97.1 |
| 39 | 80.5 | 95.3 |
| 40 | 50.2 | 98.2 |
| 41 | 77.3 | 99.5 |
| 42 | 95.7 | 99.6 |
| 43 | 39.6 | 97.0 |
| 44 | 48.7 | 97.9 |
| 45 | 80.5 | 95.3 |
| 46 | 130.5 | 98.3 |
| 47 | 121.4 | 98.6 |
| 48 | 72.2 | 94.5 |
| 49 | 53.6 | 92.1 |
| 50 | 32.5 | 96.0 |
| 51 | 60.6 | 96.8 |
| 52 | 39.4 | 97.9 |
| 53 | 18.7 | 96.5 |
| 54 | 7.9 | 97.3 |
| 55 | 17.55 | 49.8 |

### Example 2: Binding of Multimeric Proteins Towards 4-1BB Determined by Surface Plasmon Resonance (SPR)

Apparent binding kinetics and affinity of exemplary multimeric proteins to human 4-11BB (hu4-1BB) were determined by surface plasmon resonance (SPR) using a Biacore 8K instrument (GE Healthcare).

The anti-human IgG Fc antibody (GE Healthcare) was immobilized on a CM5 sensor chip using standard amine chemistry: the carboxyl groups on the chip were activated using 1-ethyl-3-(3-dimethylaminopropyl)-carbodiimide (EDC) and N-hydroxysuccinimide (NHS). Subsequently, anti-human IgG Fc antibody solution (GE Healthcare) at a concentration of 25 μg/mL in 10 mM sodium acetate (pH 5.0) was applied at a flow rate of 5 μL/min until an immobilization level of 4000-10000 resonance units (RU) was achieved. Residual non-reacted NHS-esters were blocked by passing a solution of 1 M ethanolamine across the surface. The reference channel was treated in an analogous manner. Subsequently, hu4-1BB-Fc (R&D Systems) at 0.3 μg/mL was captured by the anti-human IgG-Fc antibody at the chip surface for 180 s at a flow rate of 10 μL/min.

For affinity determination, dilutions of each testing multimeric polypeptide at various concentrations, ranging from 8-2000 nM, were prepared in HBS-EP+ buffer and applied to the prepared chip surface for affinity measurement to human 4-11BB. The binding assay was carried out with a contact time of 180 s, a dissociation time of 1200 s or 3000 s and a flow rate of 30 μL/min. All measurements were performed at 25° C. Lipocalin mutein SEQ ID NO: 64 as included in the multimeric proteins was also tested as a negative control. Regeneration of the chip surface was achieved with injections of 3 M MgCl₂ for 120 s at a flow rate of 10 μL/min followed by an extra wash with running buffer (HBS-EP+ buffer). Prior to the protein measurements, three startup cycles were performed for conditioning purposes. Data were evaluated with Biacore Evaluation software. Double referencing was used and the 1:1 binding model was used to fit the raw data.

All values are determined in an assay for multivalent interactions and the resulting data show clear multivalent interactions for the analyzed multimeric proteins and do not follow a 1:1 binding behavior. Nevertheless, data were analyzed using a 1:1 binding model to allow for an approximate comparison. In this regard, the determined kₒₙ, k_{off}, and equilibrium dissociation constant (K_{D}) (Table 2) are apparent values specific for this described assay.

Tested multimeric proteins (SEQ ID NOs: 38-54) bind hu4-1BB with higher affinity (lower K_{D} values) compared to the monomeric lipocalin mutein SEQ ID NO: 64 as included in the multimeric proteins, suggesting avidity effect.

TABLE 2
Apparent kinetic constants and apparent affinities of multimeric
proteins to human 4-1BB determined by an SPR assay

| SEQ ID | kₒₙ | k_{off} | K_{D} |
|---|---|---|---|
| NO | [M⁻¹ × s⁻¹] | [s⁻¹] | [nM] |
| 64 | 9.27E+04 | 7.08E−05 | 0.76 |
| 38 | 8.92E+04 | 2.22E−05 | 0.25 |
| 39 | 6.88E+04 | 2.63E−05 | 0.38 |
| 40 | 8.23E+04 | 9.75E−05 | 0.18 |
| 41 | 6,62E+04 | 2.07E−05 | 0.31 |
| 42 | 6.13E+04 | 1.74E−05 | 0.28 |
| 43 | 1.14E+05 | 4.42E−05 | 0.39 |
| 44 | 8.75E+04 | 2.07E−05 | 0.24 |
| 46 | 1.33E+05 | 3.18E−05 | 0.24 |
| 47 | 9.87E+04 | 6.52E−05 | 0.66 |
| 48 | 8.16E+04 | 2.76E−05 | 0.34 |
| 49 | 9.02E+04 | 1.91E−05 | 0.21 |
| 50 | 7.88E+04 | 1.36E−05 | 0.17 |
| 51 | 7.10E+04 | 2.53E−05 | 0.36 |
| 52 | 9.04E+04 | 2.19E−05 | 0.24 |
| 53 | 7.71E+04 | 9.99E−06 | 0.13 |
| 54 | 7.79E+04 | 1.99E−05 | 0.26 |
| 55 | 8.40E+04 | 5.99E−05 | 0.71 |

### Example 3. Binding of Multimeric Proteins Towards 4-1BB or GPC3 in Enzyme-Linked Immunosorbent Assay (ELISA)

An enzyme-linked immunosorbent assay (ELISA) was employed to determine the binding potency of exemplary multimeric proteins to human 4-1BB or to human GPC3.

Recombinant hu4-1BB-His (human 4-1BB with a C-terminal polyhistidine tag, R&D Systems) at the concentration of 1 μg/mL in PBS was coated overnight on microtiter plates at 4° C. After washing with PBS-0.05% T (PBS supplemented with 0.05% (v/v) Tween 20), the plates were blocked with 2% BSA (w/v) in PBS-0.1% T (PBS supplemented with 0.1% (v/v) Tween 20) for 1 h at room temperature. After washing with 100 μL PBS-0.05% T five times, exemplary multimeric proteins (SEQ ID NOs: 38-44 and 46-53) or 4-1BB-specific lipocalin mutein as included in the multimeric protein (SEQ ID NO: 64) at different concentrations, ranging from 100 to 0.002 nM, were added to the wells and incubated for 1 h at room temperature, followed by another wash step. Bound molecules under study were detected by incubation with 1:1000 diluted anti-NGAL-HRP in PBS-0.1% T-2% BSA. After an additional wash step, fluorogenic HRP substrate (QuantaBlu, Thermo) was added to each well and the fluorescence intensity was detected using a fluorescence microplate reader.

The same ELISA setup was also employed to determine the binding potency of exemplary bispecific multimeric proteins (SEQ ID NO: 54 and SEQ ID NO: 55) to GPC3, where huGPC3-His (human GPC3 with C-terminal polyhistidine tag, R&D Systems) was instead coated on a microtiter plate. The testing agents were similarly titrated and bound agents detected.

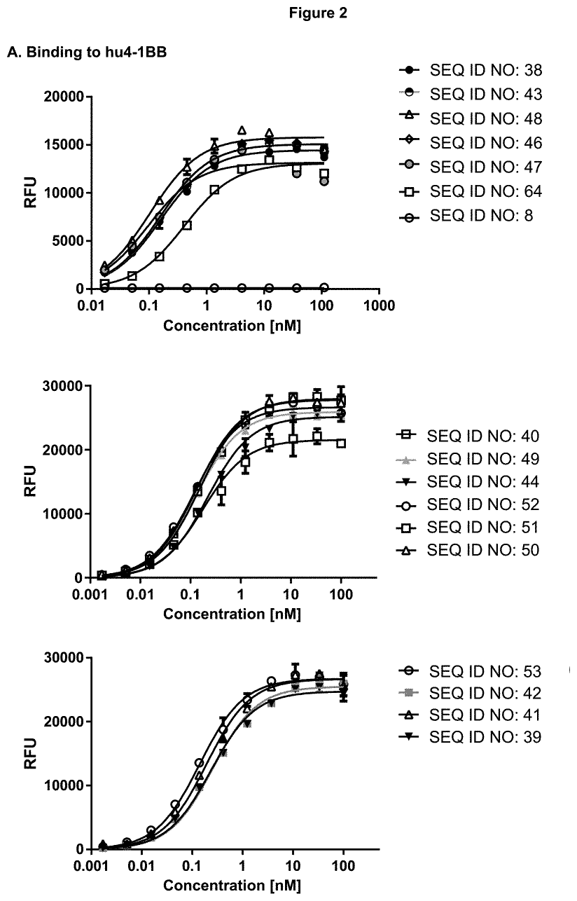

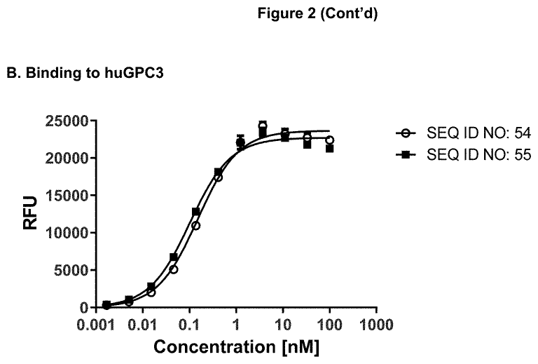

The results of exemplary experiments are depicted in FIG. 2, together with the fit curves resulting from a 1:1 binding sigmoidal fit, where the EC₅₀ value and the maximum signal were free parameters, and the slope was fixed to one. The resulting EC₅₀ values are provided in Table 3. The observed EC₅₀ values toward the hu4-1BB of all tested multimeric proteins were in the sub nanomolar range.

TABLE 3
ELISA data for 4-1BB or GPC3 binding

| SEQ ID | EC₅₀ [nM] | EC₅₀ [nM] |
|---|---|---|
| NO | Binding to hu4-1BB | Binding to huGPC3 |
| 64 | 0.41 | n.d. |
| 38 | 0.16 | n.d. |
| 39 | 0.24 | n.d. |
| 40 | 0.18 | n.d. |
| 41 | 0.19 | n.d. |
| 42 | 0.26 | n.d. |
| 43 | 0.16 | n.d. |
| 44 | 0.22 | n.d. |
| 46 | 0.16 | n.d. |
| 47 | 0.1 | n.d. |
| 48 | 0.10 | n.d. |
| 49 | 0.13 | n.d. |
| 50 | 0.13 | n.d. |
| 51 | 0.15 | n.d. |
| 52 | 0.12 | n.d. |
| 53 | 0.14 | n.d. |
| 54 | n.d. | 0.15 |
| 55 | n.d. | 0.1 |

### Example 4. Simultaneous Binding of Bispecific Multimeric Proteins to GPC3 and 4-11BB

In order to demonstrate the simultaneous binding of exemplary bispecific multimeric proteins to GPC3 and 4-1BB, a dual-binding ELISA format was used.

Recombinant huGPC3-His (human GPC3 with C-terminal polyhistidine tag, R&D Systems) at the concentration of 1 μg/mL in PBS was coated overnight on microtiter plates at 4° C. After washing with PBS-0.05% T (PBS supplemented with 0.05% (v/v) Tween 20), the plates were blocked with 2% BSA (w/v) in PBS-0.1% T (PBS supplemented with 0.1% (v/v) Tween 20) for 1 h at room temperature. After washing with 100 μL PBS-0.05% T five times, exemplary multimeric proteins (SEQ ID NOs: 54 and 55) at different concentrations, ranging from 100 to 0.002 nM, were added to the wells and incubated for 1 h at room temperature, followed by another wash step. Bound molecules under study were detected by incubation with 1 μg/mL recombinant hu4-1BB-His (biotinylated human 4-1BB with C-terminal polyhistidine tag, Sino Biological) in PBS-0.1% T-2% BSA for 1 h. This step was followed by a further wash step and incubation with 1:5000 diluted Extravidin-HRP in PBS-0.1% T-2% BSA. After an additional wash step, fluorogenic HRP substrate (QuantaBlu, Thermo) was added to each well and the fluorescence intensity was detected using a fluorescence microplate reader.

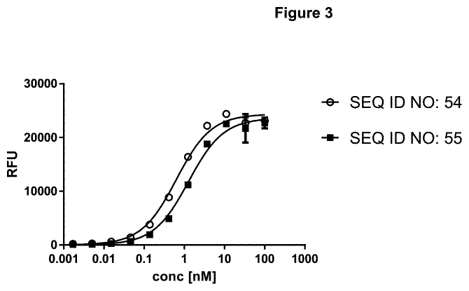

The results of exemplary experiments are depicted in FIG. 3, together with the fit curves resulting from a 1:1 binding sigmoidal fit, where the EC₅₀ value and the maximum signal were free parameters, and the slope was fixed to one. The resulting EC₅₀ values are provided in Table 4. The bispecific multimeric proteins (SEQ ID NO: 54 and SEQ ID NO: 55) show clear binding signals, demonstrating that they can engage GPC3 and 4-1BB simultaneously.

TABLE 4
ELISA data for simultaneous target
binding of both GPC3 and 4-1BB

| SEQ ID | EC₅₀ [nM] |
|---|---|
| NO | GPC3 capture_4-1BB detection |
| 54 | 0.63 |
| 55 | 1.3 |

### Example 5. Flow Cytometric Analysis of Multimeric Proteins Binding to Cells Expressing 4-11BB and GPC3

Target specific binding of multimeric proteins to 4-1BB- and GPC3-expressing cells was assessed by flow cytometry.

CHO cells were stably transfected with human 4-1BB, cynomolgus 4-1BB, or a mock control using the Flp-In system (Life Technologies) according to the manufacturer's instructions. Transfected CHO cells were maintained in Ham's F12 medium (Life Technologies) supplemented with 10% Fetal Calf Serum (Biochrom) and 500 μg/ml Hygromycin B (Roth). Cells were cultured in cell culture flasks according to manufacturer's instruction (37° C., 5% CO₂ atmosphere).

GPC3-positive tumor cell line HepG2 was cultured in Dulbecco's Modified Eagle's Medium (DMSO, Pan Biotech) supplemented with 10% Fetal Calf Serum (Sigma-Aldrich) and in cell culture flasks according to manufacturer's instruction (37° C., 5% CO₂ atmosphere).

For flow cytometric analysis, respective cell lines were incubated with testing multimeric proteins and detected using a fluorescently labeled rabbit anti-NGAL-scaffold antibody (or anti-human IgG antibody for reference antibodies) in FACS analysis as described in the following:

5×10⁴ cells per well were incubated for 1 h in ice-cold PBS containing 5% fetal calf serum (PBS-FCS). A dilution series of the tested multimeric proteins (SEQ ID NOs: 38-44 and 46-55), the 4-1BB-targeting lipocalin mutein as included in the multimeric proteins (SEQ ID NO: 64) or the GPC3-targeting lipocalin SEQ ID NO: 90 were added to the cells and incubated for 1 h on ice. Cells were washed twice with PBS and then incubated with a rabbit anti-NGAL or a goat anti-human IgG labeled with fluorescent dye Alexa 488 for 30 min on ice. Human IgG4 isotype (SEQ ID NOs: 29 and 30) and lipocalin mutein SEQ ID NO: 8 were tested as a negative control. Cells were subsequently washed and analyzed using iQue Flow cytometer (Intellicyte Screener). Mean geometric fluorescent signals were plotted and fitted with Graphpad software using nonlinear regression (shared bottom, four parameters, variable slope).

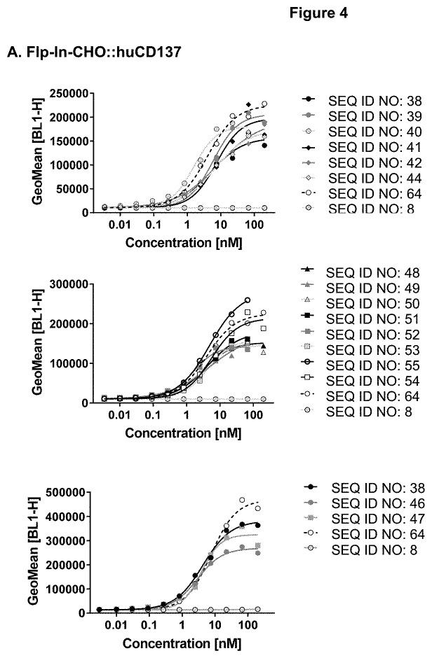

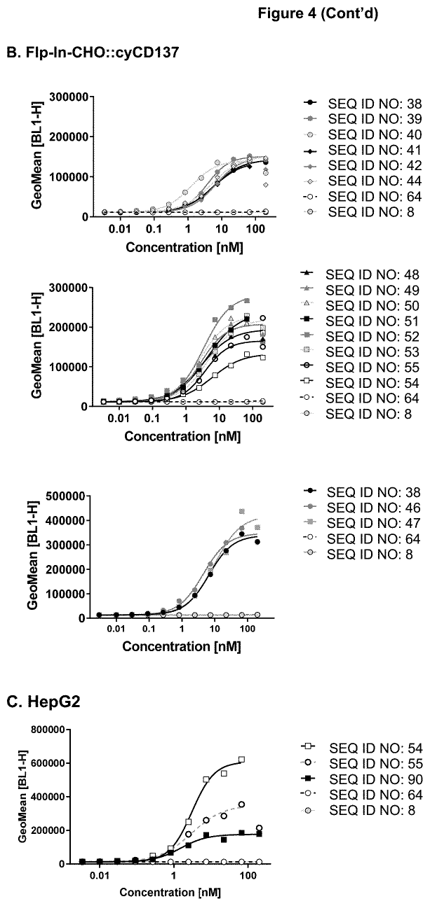

The ability of multimeric proteins to bind human 4-1BB, cynomolgus 4-1BB, and GPC3 is depicted in FIG. 4. Binding affinities (EC₅₀s, depicted in Table 5) of all tested multimeric proteins to human and cynomolgus 4-1BB-expressing cells are in the single digit nanomolar range. The results demonstrate avidity effect of the multimeric proteins as the monomeric 4-1BB specific lipocalin mutein SEQ ID NO: 64 as included in the multimeric protein is not cyno-crossreactive. No binding to the mock transfected cells was observed (data not shown).

TABLE 5
Binding affinities of the multimeric proteins
to cells expressing 4-1BB or GPC3

| SEQ ID | EC₅₀ [nM] | EC₅₀ [nM] | EC₅₀ [nM] |
|---|---|---|---|
| NO | Flp-In-CHO::hu4-1BB | Flp-In-CHO::cyno4-1BB | HepG2 |
| 64 | 3.99 | No binding | N/A |
| 38 | 4.89 | 5.92 | N/A |
| 39 | 5.87 | 3.68 | N/A |
| 40 | n.d | n.d | N/A |
| 41 | 7.37 | 5.63 | N/A |
| 42 | 8.50 | 6.59 | N/A |
| 44 | 6.64 | 5.03 | N/A |
| 45 | n.d | n.d | N/A |
| 46 | 2.89 | 4.46 | N/A |
| 47 | 3.81 | 3.81 | N/A |
| 48 | 2.98 | 2.83 | N/A |
| 49 | 4.99 | 2.75 | N/A |
| 50 | 2.49 | 2.62 | N/A |
| 51 | 4.41 | 4.41 | N/A |
| 52 | 1.50 | 3.36 | N/A |
| 53 | 4.20 | 4.55 | N/A |
| 54 | 5.49 | 5.99 | 3.07 |
| 55 | 4.08 | 6.62 | 2.98 |
| 90 | N/A | N/A | 1.53 |

### Example 6. Assessment of T Cell Activation

T cell co-stimulation by the multimeric proteins was analyzed using a T cell activation assay. Multimeric proteins were applied at different concentrations to anti-CD3 and anti-CD28 stimulated T cells, co-cultured with mock transfected Flp-In-CHO cells. IL-2 secretion levels were measured in the supernatants.

PBMCs from healthy volunteer donors were isolated from buffy coats by centrifugation through a polysucrose density gradient (Biocoll, 1.077 g/mL, Biochrom), following Biochrom's protocols. T lymphocytes were further purified from PBMC by magnetic cell sorting using a Pan T cell purification Kit (Miltenyi Biotec GmbH) following the manufacturer's instructions. Purified Pan T cells were resuspended in a buffer consisting of 90% FCS and 10% DMSO, immediately frozen down and stored in liquid nitrogen until further use. For the assay, T cells were thawed and rested in culture media (RPMI 1640, Life Technologies) supplemented with 10% FCS and 1% Penicillin-Streptomycin (Life Technologies) overnight at 37° C. in a humidified 5% CO₂ atmosphere.

The following procedure was performed using triplicates for each experimental condition: flat-bottom tissue culture plates were pre-coated with 0.25 μg/mL anti-CD3 antibody for 2 h at 37° C. and then washed twice with PBS. Mock transfected Flp-In-CHO cells were treated for 30 min with 30 μg/ml mitomycin C (Sigma Aldrich) in order to block proliferation. Mitomycin treated cells were then washed twice with PBS and plated at 8.3×10³ cells per well in culture medium to allow adhesion overnight at 37° C. in a humidified 5% CO₂ atmosphere. The CHO cells had before been grown under standard conditions, detached using Accutase (PAA Laboratories), and resuspended in culture media.

On the next days, 2.5×10⁴ T cells per well were added to the CHO cells. A dilution series of tested multimeric proteins (SEQ ID NOs: 38, 43, 46-48, and 50-53), reference 4-1BB antibody (SEQ ID NOs: 72 and 73), a bispecific hexavalent protein with trivalent targeting 4-1BB and another trivalent T cell co-stimulatory receptor targeting moiety, a negative lipocalin mutein control (SEQ ID NO: 8), or a human IgG4 isotype control (SEQ ID NOs: 29 and 30), typically ranging from 0.008 nM to 500 nM, were added to corresponding wells, followed by the additional of 0.05 μg/mL anti-CD28 antibody. The bispecific hexavalent protein comprises a 4-1BB targeting lipocalin moiety N terminally fused to a trimerization domain as shown in SEQ ID NO: 38, which is C-terminally fused to a T cell co-stimulatory receptor targeting lipocalin mutein via the linker of SEQ ID NO: 12 (L1), and which is further C-terminally fused to a linker and a Myc-His tag as shown in SEQ ID NO: 131 (L19-Myc-His). The general structure of the bispecific hexavalent protein is shown in FIG. 1 c. Plates were covered with a gas permeable seal and incubated at 37° C. in a humidified 5% CO₂ atmosphere for 3 days.

After 3 days of co-culturing, IL-2 levels in the supernatant were assessed using the human IL-2 DuoSet kit (R&D Systems) as described in the following procedures.

384 well plates were coated for 2 h at room temperature with 1 μg/mL “Human IL-2 Capture Antibody” in PBS. Subsequently, wells were washed 5 times with 80 μl PBS-0.05% T. After 1 h blocking in PBS-0.05% T containing 1% casein (w/w), assay supernatants and a concentration series of IL-2 standard diluted in culture medium was transferred to respective wells and incubated overnight at 4° C. The next day, a mixture of 100 ng/mL goat anti-hIL-2-Bio detection antibody (R&D Systems) and 1 μg/mL Sulfotag-labelled streptavidin (Mesoscale Discovery) in PBS-0.05% T containing 0.5% casein were added and incubated at room temperature for 1 h. After washing, 25 μL reading buffer (Mesoscale Discovery) was added to each well and the resulting electrochemiluminescence (ECL) signal was detected by a Mesoscale Discovery reader. Analysis and quantification were performed using Mesoscale Discovery software.

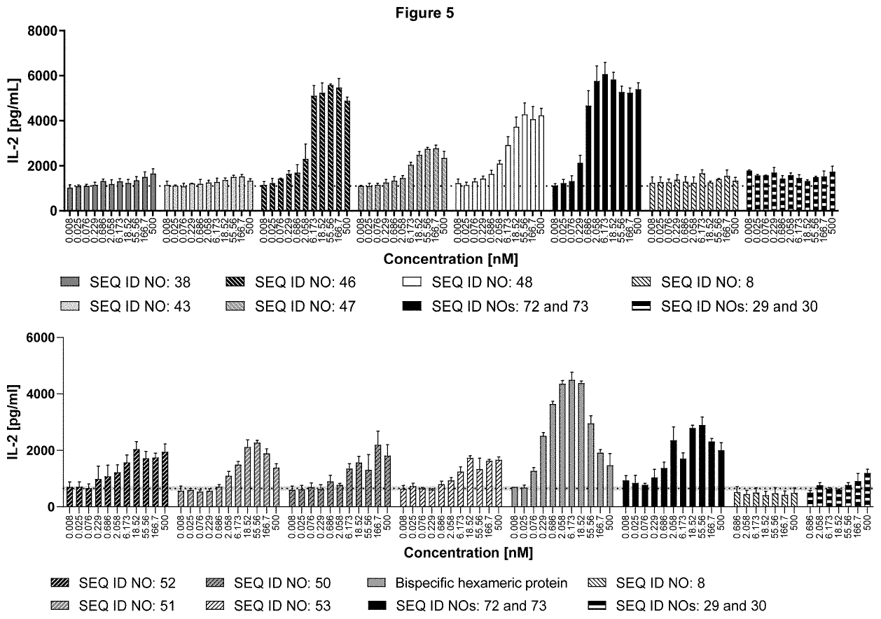

Exemplary data are shown in FIG. 5. Co-culturing of Pan T cells with CHO cells in presence of the multimeric proteins that are trivalent (SEQ ID NO: 38 and SEQ ID NO: 43) did not increase IL-2 secretion over background. Multimeric proteins that have higher valencies than trivalency (SEQ ID NO: 46-48 and 50-53) led to clear increase in IL-2 secretion compared to hlgG4 isotype control, with potencies comparable to the reference 4-11BB antibody (SEQ ID NOs: 72 and 73). In addition, the further tested bispecific hexavalent protein with trivalent targeting 4-1BB and another trivalent T cell co-stimulatory receptor targeting moiety was even more potent than the reference 4-1BB antibody (SEQ ID NOs: 72 and 73).

### Example 7. Assessment of T Cell Activation in the Presence of Tumor Cells Expressing GPC3

A T cell assay was employed to assess the ability of exemplary 4-1BB- and GPC3-bispecific multimeric proteins to co-stimulate T cell activation in a GPC3 target dependent manner. Multimeric proteins were applied at different concentrations to anti-CD3 and anti-CD28 stimulated T cells, in the presence of GPC3-positive tumor cell line HepG2. IL-2 secretion levels were measured in the supernatants.

For this assay, the same protocol was used as described in Example 5 with the exception that GPC3 positive tumor cell line HepG2 was treated with mitomycin C and used for co-culture with T cells to evaluate GPC3 target-dependent clustering of 4-1BB on T cells.

A dilution series of tested multimeric proteins (SEQ ID NO: 54 or SEQ ID NO: 55), a bispecific hexavalent protein with trivalent targeting 4-1BB and another trivalent T cell co-stimulatory receptor targeting moiety as described in Example 6, the 4-1BB-specific lipocalin mutein as included in the multimeric protein (SEQ ID NO: 64), GPC3-specific lipocalin mutein SEQ ID NO: 90, GPC3 antibody SEQ ID NOs: 108 and 109, a reference 4-1BB antibody (SEQ ID NOs: 72 and 73), human IgG4 isotype control (SEQ ID NOs: 29 and 30), or a negative control lipocalin mutein (SEQ ID NO: 8), typically ranging from 0.01 nM to 200 nM, were added to corresponding wells. Read-out was performed after incubation at 37° C. in a humidified 5% CO₂ atmosphere for 3 days. IL-2 levels in the supernatant were assessed using the human IL-2 DuoSet kit (R&D Systems) as described in Example 6.

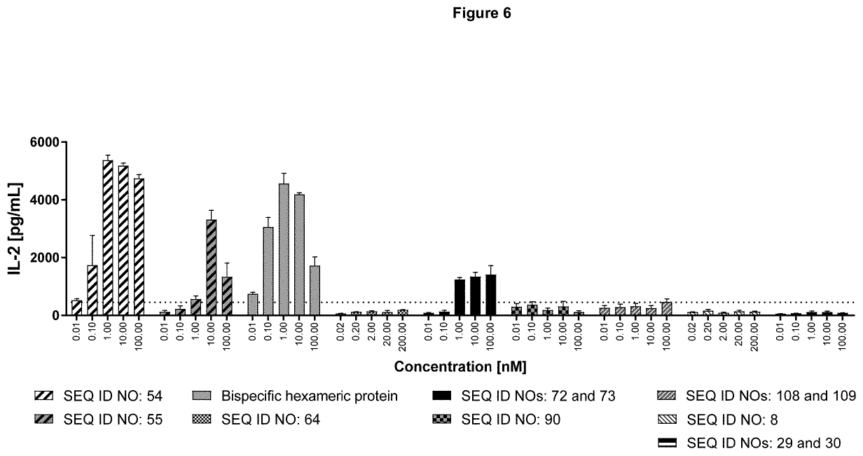

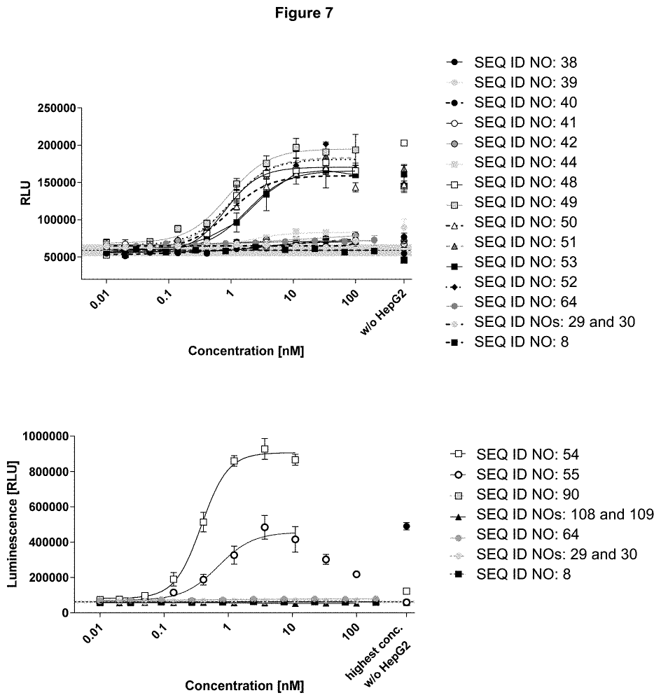

Exemplary data are shown in FIG. 6. The 4-1BB- and GPC3-bispecific multimeric proteins SEQ ID NO: 54 and SEQ ID NO: 55 as well as the bispecific hexavalent protein with trivalent targeting 4-1BB and another trivalent T cell co-stimulatory receptor targeting moiety lead to a strong increase in IL-2 secretion, which is stronger compared to the reference 4-1BB antibody SEQ ID NOs: 72 and 73. No increase of IL-2 secretion over background is observed for the reference GPC3 antibody SEQ ID NOs: 108 and 109, GPC3-specific lipocalin mutein SEQ ID NO: 90 or the 4-1BB-specific lipocalin mutein as included in the multimeric protein (SEQ ID NO:64).

### Example 8. GPC3 Dependent T Cell Co-Stimulation of the Multimeric Proteins Using a 4-11BB Bioassay

The potential of selected multimeric proteins to induce activation of 4-1BB signaling pathway in a GPC3-dependent mannerwas assessed using a commercially available double stable transfected Jurkat cell line expressing 4-1BB and the luc2 gene (humanized version of firefly luciferase) whereas luc2 expression is driven by a NFκB-responsive element. In this bioassay, 4-1BB engagement results in 4-1BB intracellular signaling, leading to NFκB-mediated luminescence.

GPC3-positive tumor cell line HepG2 was cultured in Dulbecco's Modified Eagle's Medium (DMSO, Pan Biotech) supplemented with 10% Fetal Calf Serum (Sigma-Aldrich). One day prior to the assay, HepG2 cells were plated at 6.25×10³ cells per well and allowed to adhere overnight at 37° C. in a humidified 5% CO₂ atmosphere. To test whether constructs are able to activate reporter cells in absence of GPC3-expressing HepG2 cells, some wells were incubated overnight only with medium.

The next day, 3.75×10⁴ NF-κB-Luc2/4-1BB Jurkat cells were added to each well, followed by the addition of various concentration, typically ranging from 0.01 nM to 100 nM, of tested multimeric proteins (38-42, 44, and 48-55), the 4-1BB-specific lipocalin mutein as included in the multimeric proteins (SEQ ID NO: 64), a GPC3-specific lipocalin mutein (SEQ ID NO: 90), a reference GPC3 antibody (SEQ ID NOs: 108 and 109), human IgG4 isotype control (SEQ ID NOs: 29 and 30), or a negative control lipocalin mutein (SEQ ID NO: 8). In addition, highest concentration of each construct was added to NF-κB-Luc2/4-1BB Jurkat cells in absence of HepG2 cells. Plates were covered with a gas permeable seal and incubated at 37° C. in a humidified 5% CO₂ atmosphere. After 4 h, Bio-Glo™ Reagent was added to each well and the bioluminescent signal was quantified using a luminometer (PHERAstar). Four-parameter logistic curve analysis was performed with GraphPad Prism@ to calculate EC₅₀ values (shared bottom, four parameters, variable slope) which are summarized in Table 6. The same experiment was performed in parallel in the absence of HepG2 cells. The assay was performed in triplicates.

The results of a representative experiment are depicted in FIG. 6. The trivalent multimeric proteins SEQ ID NOs: 38-42 and 44 do not induce 4-1BB mediated T cell co-stimulation in the presence and absence of GPC3. Hexavalent multimeric proteins SEQ ID NOs: 48-53 show comparable activation in the presence and absence of GPC3. Bispecific multimeric proteins SEQ ID NO: 54 and SEQ ID NO: 55 induce 4-1BB mediated T cell co-stimulation only in presence of GPC3-positive HepG2 cells, demonstrating a GPC3-dependent mode of action.

TABLE 6
Assessment of T cell activation using a 4-1BB bioassay

| SEQ ID | EC₅₀ [nM] | EC₅₀ [nM] |
|---|---|---|
| NO | Without HepG2 cells | With HepG2 cells |
| 64 | No activation | No activation |
| 38 | No activation | No activation |
| 39 | No activation | No activation |
| 40 | No activation | No activation |
| 41 | No activation | No activation |
| 42 | No activation | No activation |
| 44 | No activation | No activation |
| 48 | Activation | 0.82 |
| 49 | Activation | 0.9 |
| 50 | Activation | 0.92 |
| 51 | Activation | 0.97 |
| 52 | Activation | 1 |
| 53 | Activation | 1.83 |
| 54 | Low activation | 0.38 |
| 55 | No activation | 0.69 |
| 108 and 109 | No activation | No activation |
| 90 | No activation | No activation |

### Example 9. Assessment of CD8 and CD4 T Cell Activation

To decipher the impact of constructs on CD4+ and CD8+ T cells co-stimulation by the multimeric proteins was analyzed using a modified T cell activation assay. Multimeric proteins were applied at different concentrations to anti-CD3 stimulated isolated CD4+ or CD8+ T cells, co-cultured with mock transfected Flp-In-CHO cells. IL-2 secretion levels were measured in the supernatants.

For this assay, the same protocol was used as described in Example 5 with the exception that either isolated CD4+ or CD8+ T cells instead of Pan T cells were used. Therefore, PBMC from healthy volunteer donors were isolated. CD4 or CD8 T lymphocytes were further purified from PBMCs by magnetic cell sorting using a CD4+ T Cell Isolation Kit or CD8 Microbeads (Miltenyi Biotec GmbH) following the manufacturer's protocol.

A dilution series of a selected multimeric protein (SEQ ID NO: 52), a bispecific hexavalent protein with trivalent targeting 4-1BB and another trivalent T cell co-stimulatory receptor targeting moiety as described in Example 6, reference 4-1BB antibody (SEQ ID Nos: 72 and 73) or a human IgG4 isotype control (SEQ ID NOs: 29 and 30), typically ranging from 0.05 nM to 500 nM, were added to corresponding wells, followed by the additional of 0.05 μg/mL anti-CD28 antibody. Plates were covered with a gas permeable seal and incubated at 37° C. in a humidified 5% CO₂ atmosphere for 2 days.

After 3 days of co-culturing, IL-2 levels in the supernatant were assessed using the human IL-2 DuoSet kit (R&D Systems) as described in Example 5.

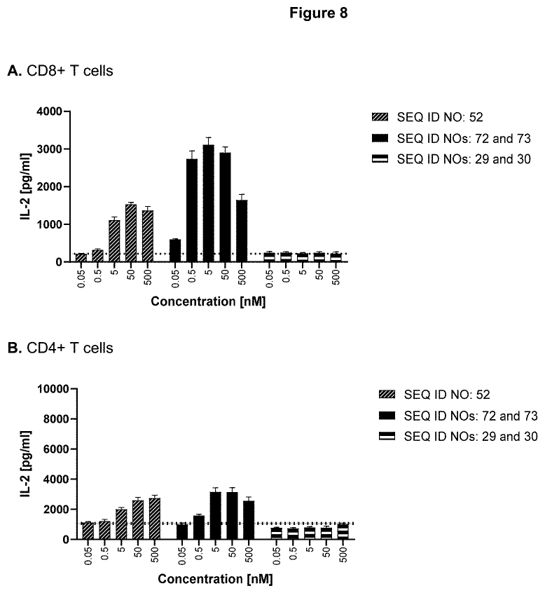

Exemplary data are shown in FIG. 8. Co-culturing of isolated CD8+ T cells with CHO cells in presence of the multimeric protein (SEQ ID NO: 52) or reference 4-1BB antibody (SEQ ID NOs: 72 and 73) led to clear increase in IL-2 secretion compared to hlgG4 isotype control (SEQ ID NOs: 29 and 30). The bispecific hexavalent protein with trivalent targeting 4-1BB and another trivalent T cell co-stimulatory receptor targeting moiety did not result in an increase of IL-2 secretion (FIG. 8A). Co-culturing of isolated CD4+ T cells with CHO cells in presence of the multimeric protein (SEQ ID NO: 52) led to clear increase in IL-2 secretion compared to hlgG4 isotype control (SEQ ID NOs: 29 and 30) with comparable potencies than reference 4-1BB antibody (SEQ ID NOs: 72 and 73). The increase of IL-2 secretion by CD4+ T cells was even stronger when using the bispecific hexavalent protein with trivalent targeting 4-1BB and another trivalent co-stimulatory receptor targeting moiety (FIG. 8B).

### Example 10. Flow Cytometric Analysis of Multimeric Proteins Binding to Cells Expressing Human 4-11BB, OX40 or PD-L1

Target-specific binding of hexavalent trimeric proteins to human 4-1BB-, OX40- or PD-L1-expressing cells was assessed by flow cytometry as described in Example 5, using CHO cells stably transfected with human 4-1BB, human OX40 or human PD-L1 (Flp-In system; Life Technologies).

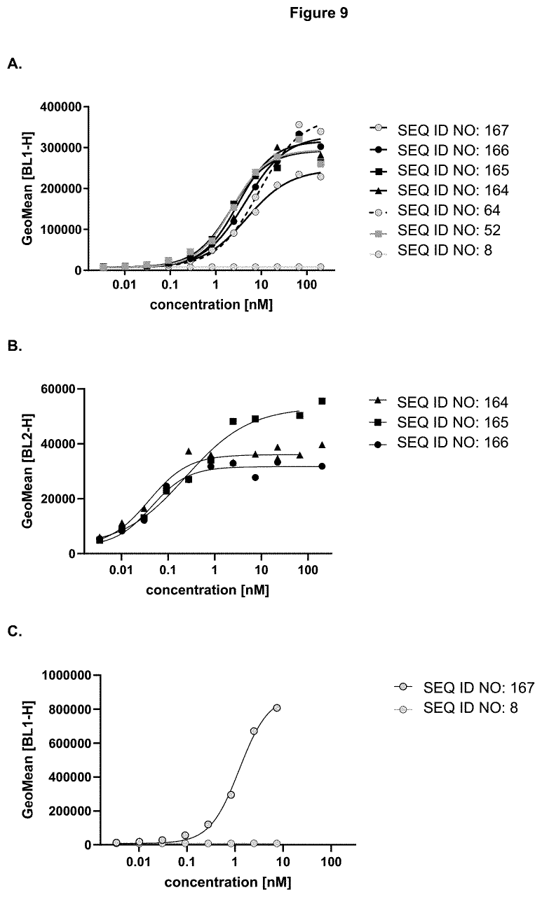

Results are shown in FIG. 9 demonstrating the ability of the multimeric proteins to bind to human 4-1BB (FIG. 9A), human OX40 (FIG. 9B), and/or human PD-L1 (FIG. 9C), respectively. Binding affinities (EC₅₀s, depicted in Table 7) of all tested multimeric proteins to target-expressing cells are in the single digit nanomolar range or even lower. No binding to mock transfected cells was observed (data not shown).

TABLE 7
Binding affinities of the multimeric proteins
to cells expressing 4-1BB, OX40 or PD-L1

| SEQ ID | EC₅₀ [nM] | EC₅₀ [nM] | EC₅₀ [nM] |
|---|---|---|---|
| NO | Flp-In-CHO::hu4-1BB | Flp-In-CHO::huOX40 | Flp-In-CHO::huPD-L1 |
| 166 | 4.51 | 0.56 | N/A |
| 165 | 2.19 | 0.18 | N/A |
| 164 | 3.03 | 0.71 | N/A |
| 167 | 4.71 | N/A | 1.23 |

### Example 11. Assessment of T Cell Activation

T cell co-stimulation by hexavalent trimeric proteins of the present disclosure was analyzed using a T cell activation assay as described in Example 6, except that Flp-In-CHO::huPD-L1 cells instead of mock transfected Flp-In-CHO cells were used.

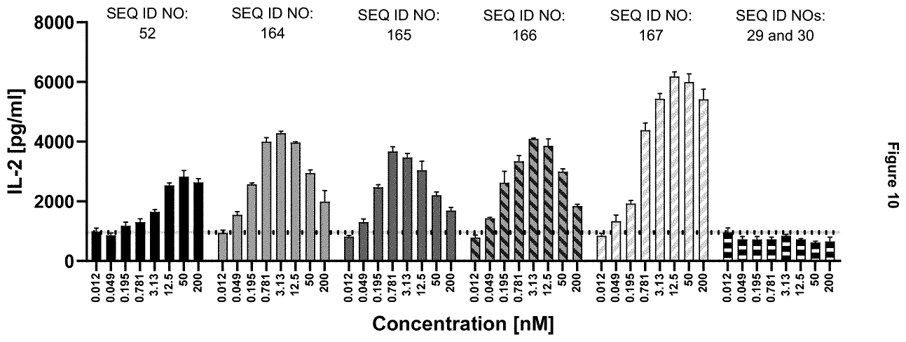

Exemplary data are shown in FIG. 10. Co-culturing of Pan T cells with CHO cells in the presence of monospecific (SEQ ID NO: 52) and bispecific (SEQ ID NOs: 164-167) hexavalent trimeric proteins led to a clear increase in IL-2 secretion compared to the hlgG4 isotype control (SEQ ID NOs: 29 and 30). The increase was significantly stronger for multimeric proteins targeting both 4-1BB and OX40 (SEQ ID NOs: 164-166) or both 4-1BB and PD-L1 (SEQ ID NO: 167) than for the multimeric protein targeting 4-1BB only (SEQ ID NO: 52).

### Example 12. Assessment of CD4 T Cell Activation

CD4+ T cell co-stimulation by hexavalent trimeric proteins of the present disclosure was analyzed using a modified T cell activation assay as described in Example 9, except that Flp-In-CHO::huPD-L1 cells instead of mock transfected Flp-In-CHO cells were used.

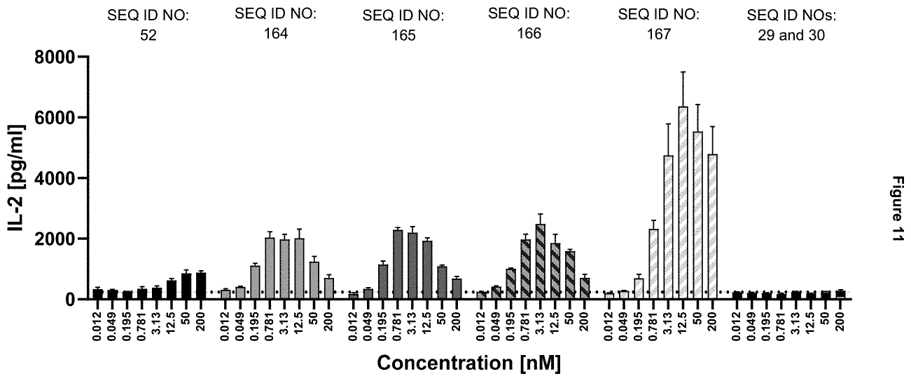

Exemplary data are shown in FIG. 11. Co-culturing of isolated CD4+ T cells with CHO cells in the presence of monospecific (SEQ ID NO: 52) and bispecific (SEQ ID NOs: 164-167) hexavalent trimeric proteins led to a clear increase in IL-2 secretion compared to the hlgG4 isotype control (SEQ ID NOs: 29 and 30). The increase was significantly stronger for multimeric proteins targeting both 4-1BB and OX40 (SEQ ID NOs: 164-166) or both 4-1BB and PD-L1 (SEQ ID NO: 167) than for the multimeric protein targeting 4-1BB only (SEQ ID NO: 52).

### Example 13. Assessment of CD8 T Cell Activation

CD8+ T cell co-stimulation by hexavalent trimeric proteins of the present disclosure was analyzed using a modified T cell activation assay as described in Example 9, except that Flp-In-CHO::huPD-L1 cells instead of mock transfected Flp-In-CHO cells were used.

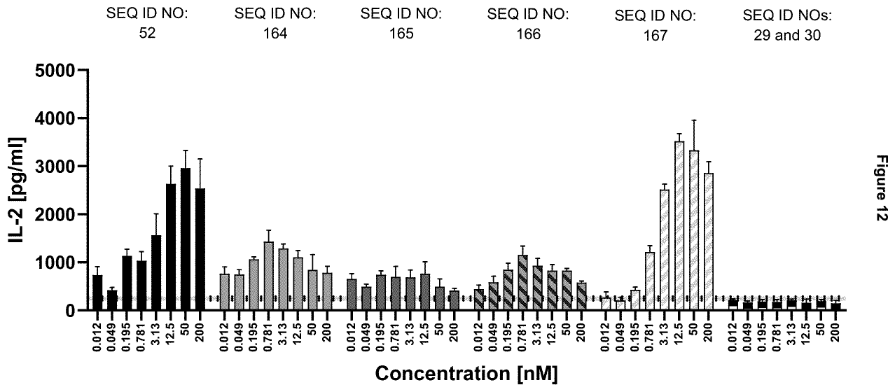

Exemplary data are shown in FIG. 12. Co-culturing of isolated CD8+ T cells with CHO cells in the presence of monospecific (SEQ ID NO: 52) and bispecific (SEQ ID NOs: 164-167) hexavalent trimeric proteins led to a clear increase in IL-2 secretion compared to the hlgG4 isotype control (SEQ ID NOs: 29 and 30). The increase was significantly stronger for the multimeric protein targeting 4-1BB only (SEQ ID NO: 52) or both 4-1BB and PD-L1 (SEQ ID NO: 167) than for multimeric proteins targeting both 4-1BB and OX40 (SEQ ID NOs: 164-166).

### Example 14. T Cell Co-Stimulation of Bispecific Hexavalent Proteins Using a 4-1BB Bioassay

The potential of selected multimeric proteins to induce activation of the 4-1BB signaling pathway was assessed using a commercially available 4-1BB bioassay as described in Example 8, except that Flp-In-CHO::huOX40 cells instead of HepG2 cells were used. The highest concentration of construct was also tested in the absence of Flp-In-CHO::huOX40 cells.

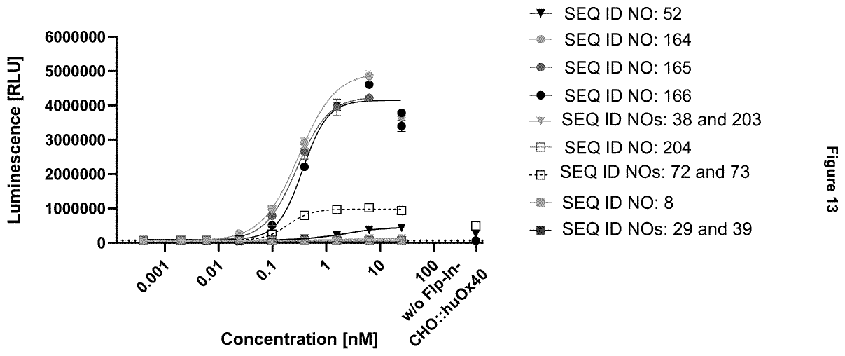

Exemplary data are shown in FIG. 13. In the presence of Flp-In-CHO::huOX40 cells, bispecific hexavalent trimeric proteins targeting 4-1BB and OX40 (SEQ ID NOs: 164-166) led to significant activation of the 4-1BB signaling pathway compared to (isotype) controls (SEQ ID NOs: 8, 29 and 30) and an OX40L protein (SEQ ID NO: 204) serving as additional negative control. The level of activation was substantially higher than the activation levels obtainable with a multimeric protein targeting 4-1BB only (SEQ ID NO: 52), with a reference 4-1BB antibody (SEQ ID NOs: 72 and 73) or with a combination of a 4-1BB-targeting trimeric protein (SEQ ID NO: 38) and an OX40-targeting trimeric protein (SEQ ID NO: 203). None of the bispecific hexavalent trimeric proteins (SEQ ID NOs: 164-166) induced 4-1BB mediated T cell co-stimulation in the absence of Flp-In-CHO::huOX40 cells.

### Example 15. T Cell Co-Stimulation of Bispecific Hexavalent Proteins Using an OX40 Bioassay

The potential of selected multimeric proteins to induce activation of the OX40 signaling pathway was assessed using a commercially available double stably transfected Jurkat cell line expressing OX40 and the luc2 gene (humanized version of firefly luciferase), wherein luc2 expression was driven by a NFκB-responsive element. In this bioassay, OX40 engagement results in OX40 intracellular signaling, leading to NFκB-mediated luminescence.

Flp-In-CHO::hu4-1BB cell line was cultured in Ham's F12 (Gibco, Thermo Fisher) supplemented with 10% Fetal Calf Serum (Sigma-Aldrich) and 500 μg/ml Hygromycin B (Carl Roth). One day prior to performing the assay, Flp-In-CHO:hu4-1BB cells were plated at 8×10³ cells per well and allowed to adhere overnight at 37° C. in a humidified 5% CO₂ atmosphere. To test whether constructs are able to activate reporter cells in absence of 4-1BB-expressing cells, some wells were incubated overnight only with medium.

The next day, 1×10⁴ NF-kB-Luc2/OX40 Jurkat cells were added to each well, followed by the addition of various concentrations, typically ranging from 0.004 nM to 100 nM, of tested 4-1BB/OX40-targeting multimeric proteins (SEQ ID NOs: 164-166), human IgG4 isotype control (SEQ ID NOs: 29 and 30), a negative control lipocalin mutein (SEQ ID NO: 8), an OX40L protein (SEQ IS NO: 204), a multimeric protein targeting 4-1BB only (SEQ ID NO: 52), a reference 4-1BB antibody (SEQ ID NOs: 72 and 73) and a combination of a 4-1BB-targeting trimeric protein (SEQ ID NO: 38) and an OX40-targeting trimeric protein (SEQ ID NO: 203). In addition, highest concentration of each construct was added to NF-kB-Luc2/OX40 Jurkat cells in absence of Flp-In-CHO::hu4-1BB cells. Plates were covered with a gas-permeable seal and incubated at 37° C. in a humidified 5% CO₂ atmosphere. After 5 h, Bio-Glo™ Reagent was added to each well and the bioluminescent signal was quantified using a luminometer (PHERAstar). Four-parameter logistic curve analysis was performed with GraphPad Prism®. The assay was performed in triplicates.

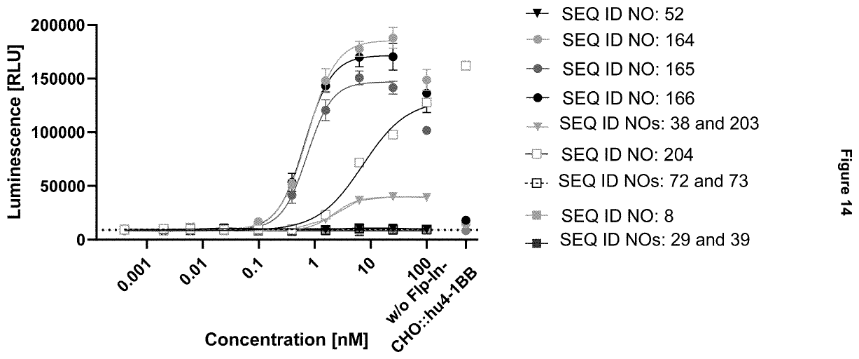

The results of a representative experiment are depicted in FIG. 14. In the presence of Flp-In-CHO::hu4-1BB cells, bispecific hexavalent trimeric proteins targeting 4-1BB and OX40 (SEQ ID NOs: 164-166) led to significant activation of the OX40 signaling pathway compared to (isotype) controls (SEQ ID NOs: 8, 29 and 30). The level of activation was substantially higher than the activation levels obtainable with the multimeric protein targeting 4-1BB only (SEQ ID NO: 52), with the reference 4-1BB antibody (SEQ ID NOs: 72 and 73), with the OX40L protein (SEQ ID NO: 204) or with the combination of a 4-1BB-targeting trimeric protein (SEQ ID NO: 38) and an OX40-targeting trimeric protein (SEQ ID NO: 203). None of the bispecific hexavalent trimeric proteins (SEQ ID NOs: 164-166) induced 4-1BB mediated T cell co-stimulation in absence of Flp-In-CHO::hu4-1BB cells.

Embodiments illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present embodiments have been specifically disclosed by preferred embodiments and optional features, modification and variations thereof may be resorted to by those skilled in the art, and that such modifications and variations are considered to be within the scope of this invention. All patents, patent applications, textbooks and peer-reviewed publications described herein are hereby incorporated by reference in their entirety. Furthermore, where a definition or use of a term in a reference, which is incorporated by reference herein is inconsistent or contrary to the definition of that term provided herein, the definition of that term provided herein applies and the definition of that term in the reference does not apply. Each of the narrower species and subgeneric groupings falling within the generic disclosure also forms part of the invention. This includes the generic description of the invention with a proviso or negative limitation removing any subject matter from the genus, regardless of whether or not the excised material is specifically recited herein. In addition, where features are described in terms of Markush groups, those skilled in the art will recognize that the disclosure is also thereby described in terms of any individual member or subgroup of members of the Markush group. Further embodiments will become apparent from the following claims.

Equivalents: Those skilled in the art will recognize, or be able to ascertain using no more than routine experimentation, many equivalents to the specific embodiments of the invention described herein. Such equivalents are intended to be encompassed by the following claims. All publications, patents and patent applications mentioned in this specification are herein incorporated by reference into the specification to the same extent as if each individual publication, patent or patent application was specifically and individually indicated to be incorporated herein by reference.

### VI. NON-PATENT REFERENCES

1. ADDIN EN.REFLIST ALTSCHUL, S. F., GISH, W., MILLER, W., MYERS, E. W. & LIPMAN, D. J. 1990. Basic local alignment search tool. J Mol Biol, 215, 403-10.
2. ALTSCHUL, S. F., MADDEN, T. L., SCHAFFER, A. A., ZHANG, J., ZHANG, Z., MILLER, W. & LIPMAN, D. J. 1997. Gapped BLAST and PSI-BLAST: a new generation of protein database search programs. Nucleic Acids Res, 25, 3389-402.
3. ALTSHULER, E. P., SEREBRYANAYA, D. V. & KATRUKHA, A. G. 2010. Generation of recombinant antibodies and means for increasing their affinity. Biochemistry (Mosc), 75, 1584-605.
4. BREUSTEDT, D. A., KORNDORFER, I. P., REDL, B. & SKERRA, A. 2005. The 1.8-A crystal structure of human tear lipocalin reveals an extended branched cavity with capacity for multiple ligands. J Biol Chem, 280, 484-93.
5. BRUCKDORFER, T., MARDER, O. & ALBERICIO, F. 2004. From production of peptides in milligram amounts for research to multi-tons quantities for drugs of the future. Curr Pharm Biotechnol, 5, 29-43.
6. CHACON, J. A., WU, R. C., SUKHUMALCHANDRA, P., MOLLDREM, J. J., SARNAIK, A., PILON-THOMAS, S., WEBER, J., HWU, P. & RADVANYI, L. 2013. Co-stimulation through 4-1BB/CD137 improves the expansion and function of CD8(+) melanoma tumor-infiltrating lymphocytes for adoptive T-cell therapy. PLoS One, 8, e60031.
7. COLE, S. P., CAMPLING, B. G., LOUWMAN, I. H., KOZBOR, D. & RODER, J. C. 1984. A strategy for the production of human monoclonal antibodies reactive with lung tumor cell lines. Cancer Res, 44, 2750-3.
8. DENNIS, M. S., ZHANG, M., MENG, Y. G., KADKHODAYAN, M., KIRCHHOFER, D., COMBS, D. & DAMICO, L. A. 2002. Albumin binding as a general strategy for improving the pharmacokinetics of proteins. J Biol Chem, 277, 35035-43.
9. FISHER, T. S., KAMPERSCHROER, C., OLIPHANT, T., LOVE, V. A., LIRA, P. D., DOYONNAS, R., BERGQVIST, S., BAXI, S. M., ROHNER, A., SHEN, A. C., HUANG, C., SOKOLOWSKI, S. A. & SHARP, L. L. 2012. Targeting of 4-1BB by monoclonal antibody PF-05082566 enhances T-cell function and promotes anti-tumor activity. Cancer Immunol Immunother, 61, 1721-33.
10. FLOWER, D. R. 1996. The lipocalin protein family: structure and function. Biochem J, 318 (Pt 1), 1-14.
11. FLOWER, D. R. 2000. Beyond the superfamily: the lipocalin receptors. Biochim Biophys Acta, 1482, 327-36.
12. FLOWER, D. R., NORTH, A. C. & SANSOM, C. E. 2000. The lipocalin protein family: structural and sequence overview. Biochim Biophys Acta, 1482, 9-24.
13. FUERTGES, F. & ABUCHOWSKI, A. 1990. The clinical efficacy of poly(ethylene glycol)-modified proteins. Journal of Controlled Release, 11, 139-148.
14. HARLOW, E. & LANE, D. 1988. Antibodies: a laboratory manual, Cold Spring Harbor, N.Y., Cold Spring Harbor Laboratory.
15. HARLOW, E. & LANE, D. 1999. Using antibodies: a laboratory manual, Cold Spring Harbor, N.Y., Cold Spring Harbor Laboratory Press.
16. HOLLIGER, P. & HUDSON, P. J. 2005. Engineered antibody fragments and the rise of single domains. Nat Biotechnol, 23, 1126-36.
17. HOLLIGER, P., PROSPERO, T. & WINTER, G. 1993. “Diabodies”: small bivalent and bispecific antibody fragments. Proc Natl Acad Sci USA, 90, 6444-8.
18. KONIG, T. & SKERRA, A. 1998. Use of an albumin-binding domain for the selective immobilisation of recombinant capture antibody fragments on ELISA plates. J Immunol Methods, 218, 73-83.
19. KOZBOR, D. & RODER, J. C. 1983. The production of monoclonal antibodies from human lymphocytes. Immunol Today, 4, 72-9.
20. LI, J., SAI, T., BERGER, M., CHAO, Q., DAVIDSON, D., DESHMUKH, G., DROZDOWSKI, B., EBEL, W., HARLEY, S., HENRY, M., JACOB, S., KLINE, B., LAZO, E., ROTELLA, F., ROUTHIER, E., RUDOLPH, K., SAGE, J., SIMON, P., YAO, J., ZHOU, Y., KAVURU, M., BONFIELD, T., THOMASSEN, M. J., SASS, P. M., NICOLAIDES, N. C. & GRASSO, L. 2006. Human antibodies for immunotherapy development generated via a human B cell hybridoma technology. Proc Natl Acad Sci USA, 103, 3557-62.
21. LI, S. Y. & LIU, Y. 2013. Immunotherapy of melanoma with the immune costimulatory monoclonal antibodies targeting CD137. Clin Pharmacol, 5, 47-53.
22. LOWMAN, H. B. 1997. Bacteriophage display and discovery of peptide leads for drug development. Annu Rev Biophys Biomol Struct, 26, 401-24.
23. MARTINET, O., DIVINO, C. M., ZANG, Y., GAN, Y., MANDELI, J., THUNG, S., PAN, P. Y. & CHEN, S. H. 2002. T cell activation with systemic agonistic antibody versus local 4-1BB ligand gene delivery combined with interleukin-12 eradicate liver metastases of breast cancer. Gene Ther, 9, 786-92.
24. MELERO, I., BACH, N., HELLSTROM, K. E., ARUFFO, A., MITTLER, R. S. & CHEN, L. 1998. Amplification of tumor immunity by gene transfer of the co-stimulatory 4-1BB ligand: synergy with the CD28 co-stimulatory pathway. Eur J Immunol, 28, 1116-21.
25. OSBORN, B. L., OLSEN, H. S., NARDELLI, B., MURRAY, J. H., ZHOU, J. X., GARCIA, A., MOODY, G., ZARITSKAYA, L. S. & SUNG, C. 2002. Pharmacokinetic and pharmacodynamic studies of a human serum albumin-interferon-alpha fusion protein in cynomolgus monkeys. J Pharmacol Exp Ther, 303, 540-8.
26. PERVAIZ, S. & BREW, K. 1987. Homology and structure-function correlations between alpha 1-acid glycoprotein and serum retinol-binding protein and its relatives. FASEB J, 1, 209-14.
27. RABU, C., QUÉMÉNER, A., JACQUES, Y., ECHASSERIEAU, K., VUSIO, P. & LANG, F. 2005. Production of Recombinant Human Trimeric CD137L (4-1BBL): CROSS-LINKING IS ESSENTIAL TO ITS T CELL CO-STIMULATION ACTIVITY. Journal of Biological Chemistry, 280, 41472-41481.
28. RODI, D. J. & MAKOWSKI, L. 1999. Phage-display technology—finding a needle in a vast molecular haystack. Curr Opin Biotechnol, 10, 87-93.
29. SAMBROOK, J. & RUSSELL, D. W. 2001. Molecular cloning: a laboratory manual, Cold Spring Harbor, N.Y., Cold Spring Harbor Laboratory Press.
30. SCHMIDT, T. G., KOEPKE, J., FRANK, R. & SKERRA, A. 1996. Molecular interaction between the Strep-tag affinity peptide and its cognate target, streptavidin. J Mol Biol, 255, 753-66.
31. SKERRA, A. 2000. Lipocalins as a scaffold. Biochim Biophys Acta, 1482, 337-50.
32. SMITH, T. F. & WATERMAN, M. S. 1981. Identification of common molecular subsequences. J Mol Biol, 147, 195-7.
33. SNELL, L. M., LIN, G. H., MCPHERSON, A. J., MORAES, T. J. & WATTS, T. H. 2011. T—cell intrinsic effects of GITR and 4-1BB during viral infection and cancer immunotherapy. Immunol Rev, 244, 197-217.
34. VAJO, Z. & DUCKWORTH, W. C. 2000. Genetically engineered insulin analogs: diabetes in the new millenium. Pharmacol Rev, 52, 1-9.
35. VENTURI, M., SEIFERT, C. & HUNTE, C. 2002. High level production of functional antibody Fab fragments in an oxidizing bacterial cytoplasm. J Mol Biol, 315, 1-8.
36. WARD, E. S., GUSSOW, D., GRIFFITHS, A. D., JONES, P. T. & WINTER, G. 1989. Binding activities of a repertoire of single immunoglobulin variable domains secreted from Escherichia coli. Nature, 341, 544-6.
37. WYZGOL, A., MULLER, N., FICK, A., MUNKEL, S., GRIGOLEIT, G. U., PFIZENMAIER, K. & WAJANT, H. 2009. Trimer stabilization, oligomerization, and antibody-mediated cell surface immobilization improve the activity of soluble trimers of CD27L, CD40L, 41BBL, and glucocorticoid-induced TNF receptor ligand. J Immunol, 183, 1851-61.
38. YANG, Y., YANG, S., YE, Z., JAFFAR, J., ZHOU, Y., CUTTER, E., LIEBER, A., HELLSTROM, I. & HELLSTROM, K. E. 2007. Tumor cells expressing anti-CD137 scFv induce a tumor-destructive environment. Cancer Res, 67, 2339-44.
39. YAO, S., ZHU, Y. & CHEN, L. 2013. Advances in targeting cell surface signalling molecules for immune modulation. Nat Rev Drug Discov, 12, 130-46.
40. YE, Q., SONG, D. G., POUSSIN, M., YAMAMOTO, T., BEST, A., LI, C., COUKOS, G. & POWELL, D. J., JR. 2014. CD137 accurately identifies and enriches for naturally occurring tumor-reactive T cells in tumor. Clin Cancer Res, 20, 44-55.
41. YE, Z., HELLSTROM, I., HAYDEN-LEDBETTER, M., DAHLIN, A., LEDBETTER, J. A. & HELLSTROM, K. E. 2002. Gene therapy for cancer using single-chain Fv fragments specific for 4-1BB. Nat Med, 8, 343-8.
42. ZHANG, H., KNUTSON, K. L., HELLSTROM, K. E., DISIS, M. L. & HELLSTROM, I. 2006. Antitumor efficacy of CD137 ligation is maximized by the use of a CD137 single-chain Fv-expressing whole-cell tumor vaccine compared with CD137-specific monoclonal antibody infusion. Mol Cancer Ther, 5, 149-55.

1. A multimeric protein comprising at least three monomer polypeptides, wherein each monomer polypeptide comprises (1) a first 4-1BB-targeting moiety (T1), and (2) an oligomerization moiety (O).

2. The multimeric protein of claim 1, wherein the first 4-1BB-targeting moiety (T1) is fused at its N-terminus or C-terminus to the C-terminus or N-terminus, respectively, of the oligomerization moiety (O) via a linker (L).

3. The multimeric protein of claim 1 or 2, wherein the monomer polypeptide comprises at least one additional targeting moiety (T2).

4. The multimeric protein of any one of claims 1-3, wherein the monomer polypeptide comprises an additional targeting moiety (T2), wherein the additional targeting moiety is placed in tandem with the first 4-1BB-targeting moiety (T1).

5. The multimeric protein of claim 4, wherein the monomer polypeptide has one of the following configurations:
a. T1-L′-T2-L-O;
b. T2-L′-T1-L-O;
c. O-L-T1-L′-T2; or
d. O-L-T2-L′-T1
wherein L′ is a linker that is the same as or different from L.

6. The multimeric protein of any one of claims 1-3, wherein the monomer polypeptide comprises an additional targeting moiety (T2), wherein the additional targeting moiety (T2) is linked to a different terminus of the oligomerization moiety (O) than the first 4-1EE-targeting moiety (T1).

7. The multimeric protein of claim 6, wherein the monomer polypeptide has one of the following configurations:
a. T1-L-O-L′-T2; or
b. T2-L′-O-L-T1
wherein L′ is a linker that is the same as or different from L.

8. The multimeric protein of any one of claims 3-7, wherein the additional targeting moiety (T2) is a second 4-1BB-targeting moiety.

9. The multimeric protein of claim 8, wherein the second 4-1BB-targeting moiety is the same as the first 4-1BB-targeting moiety (T1).

10. The multimeric protein of any one of claims 3-7, wherein the additional targeting moiety (T2) is a moiety that targets a tumor associated antigen.

11. The multimeric protein of any one of claims 3-7, wherein the additional targeting moiety (T2) is a moiety that targets a tumor associated antigen and is a lipocalin mutein.

12. The multimeric protein of any one of claims 3-7, wherein the additional targeting moiety (T2) is a moiety that targets a tumor associated antigen and is an antibody or an antigen-binding domain or derivative thereof.

13. The multimeric protein of any one of claims 3-7, wherein the additional targeting moiety (T2) is a moiety that targets a tumor associated antigen and is a single chain variable fragment (scFv).

14. The multimeric protein of any one of claims 3-7, wherein the additional targeting moiety (T2) is a GPC3-targeting moiety.

15. The multimeric protein of any one of claims 3-7, wherein the additional targeting moiety (T2) is a GPC3-targeting moiety that is a lipocalin mutein.

16. The multimeric protein of claim 15, wherein the lipocalin mutein has at least 85% sequence identity to an amino acid sequence selected from the group consisting of SEQ ID NOs: 74-97.

17. The multimeric protein of any one of claims 3-7, wherein the additional targeting moiety (T2) is a GPC3-targeting moiety that is an antibody or an antigen-binding domain or derivative thereof.

18. The multimeric protein of any one of claims 3-7, wherein the additional targeting moiety (T2) is a GPC3-targeting moiety that is a single chain variable fragment (scFv).

19. The multimeric protein of any one of claims 3-7, wherein the additional targeting moiety (T2) is a PD-L1-targeting moiety.

20. The multimeric protein of any one of claims 3-7, wherein the additional targeting moiety (T2) is a PD-L1-targeting moiety that is a single chain variable fragment (scFv).

21. The multimeric protein of claim 20, wherein the scFv has at least 85% sequence identity to the amino acid sequence shown in SEQ ID NO: 172.

22. The multimeric protein of any one of claims 3-7, wherein the additional targeting moiety (T2) is a T cell activation enhancing targeting moiety.

23. The multimeric protein of any one of claims 3-7, wherein the additional targeting moiety (T2) is a T cell activation enhancing targeting moiety that is a lipocalin mutein.

24. The multimeric protein of any one of claims 3-7, wherein the additional targeting moiety (T2) is a T cell activation enhancing targeting moiety that is an antibody or an antigen-binding domain or derivative thereof.

25. The multimeric protein of any one of claims 3-7, wherein the additional targeting moiety (T2) is a T cell activation enhancing targeting moiety that is a single chain variable fragment (scFv).

26. The multimeric protein of any one of claims 3-7, wherein the additional targeting moiety (T2) is an OX40-targeting moiety.

27. The multimeric protein of any one of claims 3-7, wherein the additional targeting moiety (T2) is an OX40-targeting moiety that is a lipocalin mutein.

28. The multimeric protein of claim 27, wherein the lipocalin mutein has at least 85% sequence identity to an amino acid sequence selected from the group consisting of SEQ ID NOs: 174-202.

29. The multimeric protein of any one of claims 1-28, wherein the first 4-1BB-targeting moiety (T1) is a lipocalin mutein.

30. The multimeric protein of any one of claims 1-29, wherein the first 4-1BB-targeting moiety (T1) is a lipocalin mutein having at least 85% sequence identity to an amino acid sequence selected from the group consisting of SEQ ID NOs: 56-71.

31. The multimeric protein of any one of claims 1-30, wherein the oligomerization moiety (O) is capable of promoting trimerization.

32. The multimeric protein of any one of claims 1-31, wherein the oligomerization moiety (O) is a trimerization domain of a collagen.

33. The multimeric protein of any one of claims 1-32, wherein the oligomerization moiety (O) has at least 85% sequence identity to an amino acid sequence selected from the group consisting of SEQ ID NOs: 35-37.

34. The multimeric protein of any one of claims 1-33, wherein the multimeric protein is a trimeric protein.

35. The multimeric protein of any one of claims 1-30 and 33, wherein the multimeric protein is a tetrameric protein.

36. The multimeric protein of any one of claims 2-35, wherein the linker (L) has at least 85% sequence identity to an amino acid sequence selected from the group consisting of SEQ ID NOs: 12-28.

37. The multimeric protein of any one of claims 1-36, wherein the multimeric protein has at least 85% sequence identity to an amino acid sequence selected from the group consisting of SEQ ID NOs: 38-55 and 164-167.

38. The multimeric protein of any one of claims 1-37, wherein the multimeric protein comprises an amino acid sequence selected from the group consisting of SEQ ID NOs: 38-55 and 164-167.

39. The multimeric protein of any one of claims 1-38, wherein the multimeric protein is capable of binding 4-1BB with a K_{D} value of about 0.68 nM or lower.

40. The multimeric protein of any one of claims 1-39, wherein the multimeric protein is capable of binding 4-1BB with an apparent K_{D} value lower than the K_{D} value of the 4-1BB-targeting lipocalin mutein that is included in the monomer polypeptide.

41. The multimeric protein of claim 39 or 40, wherein the apparent K_{D} value is determined by surface plasmon resonance (SPR).

42. The multimeric protein of any one of claims 1-41, wherein the multimeric protein is cross-reactive with cynomolgus 4-11BE.

43. A nucleic acid molecule comprising a nucleotide sequence encoding a monomer polypeptide comprised in a multimeric protein of any one of claims 1-42.

44. The nucleic acid molecule of claim 43, wherein the nucleic acid molecule is operably linked to a regulatory sequence to allow expression of said nucleic acid molecule.

45. The nucleic acid molecule of claim 43 or 44, wherein the nucleic acid molecule is comprised in a vector or in a phagemid vector.

46. The nucleic acid molecule of any one of claim 43 or 44, wherein the nucleic acid molecule is comprised in a viral vector, in a nanoparticle, or a liposome.

47. The nucleic acid molecule of any one of claims 43-46, wherein the nucleic acid molecule is comprised in the genomic DNA of a host cell.

48. A cell containing a nucleic acid molecule of any one of claims 43-47 and/or expressing the multimeric protein of any one of claims 1-42 and/or expressing a monomer polypeptide as defined in any one of claims 1-42.

49. The cell of claim 48, wherein the cell secretes the multimeric protein and/or the monomer polypeptide.

50. The cell of claim 48, wherein the cell secretes the monomer polypeptide.

51. The cell of claim 50, wherein the monomer polypeptide self-assembles to a multimeric protein after secretion.

52. The cell of any one of claims 48-51, wherein the cell is an immune cell.

53. The cell of claim 52, wherein the cell is a T cell.

54. The cell of claim 53, wherein the cell is a CD8+ T cell.

55. The cell of claim 53, wherein the cell is a CD4+ T cell.

56. The cell of any one of claims 52-55, wherein the cell comprises a recombinant antigen receptor.

57. The cell of claim 56, wherein the recombinant antigen receptor is a chimeric antigen receptor (CAR).

58. The cell of claim 56, wherein the recombinant antigen receptor is a T cell receptor (TCR).

59. The cell of any one of claims 52-57, wherein the cell is a CAR-T cell.

60. The cell of any one of claims 52-59, wherein the cell expresses 4-11BE.

61. The cell of any one of claims 52-60, wherein the cell is a human cell.

62. A method of producing the multimeric protein of any one of claims 1-42, wherein the multimeric protein is produced starting from the nucleic acid coding for the monomer polypeptides comprised in the multimeric protein.

63. The method of claim 62, wherein the multimeric protein is produced in a bacterial or eukaryotic host organism.

64. A use of the multimeric protein of any one of claims 1-42 or a composition comprising such multimeric protein or a cell of any one of claims 48-61 for inducing 4-1BB (and/or, optionally, OX40) clustering and activation on T cells.

65. A use of the multimeric protein of any one of claims 1-42 or a composition comprising such multimeric protein or a cell of any one of claims 48-61 for co-stimulating T cells and/or activating downstream signaling pathways of 4-1BB (and/or, optionally, OX40).

66. A use of the multimeric protein of any one of claims 1-42 or a composition comprising such multimeric protein or a cell of any one of claims 48-61 for co-stimulating T cells when engaging GPC3-expressing or PD-L1-expressing tumor cells.

67. The use of any one of claims 64-66, wherein the T cell is a T cell expressing the multimeric protein and/or one of its monomer polypeptides.

68. The use of any one of claims 64-66, wherein the T cell is a T cell not expressing the multimeric protein and/or one of its monomer polypeptides.

69. A pharmaceutical composition comprising one or more multimeric proteins of any one of claims 1-42 and/or one or more cells of any one of claims 48-61.

70. The multimeric protein of any one of claims 1-42 and/or the cell of any one of claims 48-61 for use in a therapy.

71. The multimeric protein and/or the cell for use of claim 70, wherein the use is in the treatment of cancer.

72. Use of a multimeric protein of any one of claims 1-42 and/or the cell of any one of claims 48-61 for the manufacture of a medicament.

73. The use of claim 72, wherein the medicament is for the treatment of cancer.
